(19) United States
(12) Patent Application Publication
(10) Pub. No.: US 2026/0345641 A1
(43) Pub. Date: Nov. 5, 2026

(54) COMPOSITIONS AND USES THEREOF

(71) Applicant: Algorae Pharmaceuticals Ltd, Melbourne, VIC (AU)

(72) Inventors: David HAINSWORTH, Kalamunda, Western Australia (AU); Brad DILKES, Brighton, Victoria (AU); James MCKENNA, Pascoe Vale South, Victoria (AU)

(21) Appl. No.: 19/516,317

(22) PCT Filed: Jul. 25, 2024
(86) PCT No.: PCT/AU2024/050791

§ 371 Date: Mar. 4, 2026

(30) Foreign Application Priority Data
Jul. 25, 2023 (AU) 2023902369
Apr. 5, 2024 (AU) 2024900956

(51) Int. Cl. A61K 31/00; A61K 31/445; A61P 25/28; G16H 20/10

(52) U.S. Cl. CPC A61K 31/658; A61K 31/445; A61P 25/28; G16H 20/10

(57) ABSTRACT

The present disclosure relates generally to pharmaceutical compositions comprising cannabidiol (CBD) or a pharmaceutically acceptable salt or derivative thereof, and an acetylcholinesterase inhibitor, and methods for the treatment or prevention of neurodegenerative disorders, dementia, or diseases associated with glutamate-mediated excitotoxicity.

### RELATED APPLICATIONS

This application claims priority from Australian Provisional Patent Application No. 2023902369 filed on 25 Jul. 2023 and Australian Provisional Patent Application No. 2024900956 filed on 5 Apr. 2024, the entire contents of which are incorporated by reference.

### FIELD

The present disclosure relates generally to pharmaceutical compositions comprising cannabidiol (CBD) or a pharmaceutically acceptable salt or derivative thereof, and an acetylcholinesterase inhibitor, and methods for the treatment or prevention of neurodegenerative disorders, dementia, or diseases associated with glutamate-mediated excitotoxicity.

### BACKGROUND

Neurodegenerative disorders are characterized by a progressive decline of the motor and/or cognitive function caused by the selective loss of neurons within the central nervous system, and include Alzheimer's disease, Parkinson's disease, Huntington's disease, and chronic traumatic encephalopathy (CTE). While patients with early-stage neurodegenerative disease (i.e., the “prodromal phase”) typically have few symptoms and only a small number of brain regions affected by protein aggregation, once the disease progresses to symptomatic stage, there is extensive damage to either vulnerable brain regions in diseases that are characterized by more focal dysfunction or widespread damage to multiple brain regions. As a consequence, by the time a patient presents with symptoms, there is already extensive neuronal loss and dystrophy. Due to the progressive nature and pathology of neurodegenerative diseases, they are also the most common cause of dementia.

Dementia, also referred to as major neurocognitive disorder (MND), is characterized by an acquired loss of cognition in multiple cognitive domains sufficiently severe to affect social or occupational function. Dementia is commonly associated with at least one neurodegenerative disorder. According to the World Health Organization, it is estimated that around 10 million people develop dementia every year, with the number of affected individuals increasing to 75 million in 2030 and 132 million by 2050.

There are no curative treatments for neurodegenerative disorders or dementia, nor are any treatments available to prevent disease progression. Current standard of care for the treatment of neurodegenerative disorders and dementia includes the amelioration of symptoms using acetylcholinesterase inhibitors (AChEIs), N-methyl-D-aspartase (NMDA) receptor antagonists, and a range of other medications to treat other chronic diseases that commonly accompany neurodegenerative disorders including hypertension, stroke, diabetes and coronary heart disease. However, these medications are not efficacious in all patients, or have only modest symptomatic effect. Moreover, the need to take multiple medications for the treatment of neurodegenerative disorders can lead to a greater risk of drug-related problems, including potentially inappropriate medications, drug-drug interactions, and adverse drug reactions. Therefore, there remains an urgent need for the development of new pharmacological approaches for the treatment or prevention of neurodegenerative disorders and dementia.

### SUMMARY

In an aspect of the present disclosure, there is provided a method for the treatment or prevention of at least one neurodegenerative disorder and/or dementia, the method comprising administering to a subject in need thereof: (i) cannabidiol (CBD) or a pharmaceutically acceptable salt or derivative thereof, and (ii) an acetylcholinesterase inhibitor, wherein the subject is administered from about 1 mg to about 1500 mg CBD or a pharmaceutically acceptable salt or derivative thereof per day.

In another aspect of the present disclosure, there is provided a use of CBD or a pharmaceutically acceptable salt or derivative thereof, and an acetylcholinesterase inhibitor in the manufacture of a medicament for the treatment or prevention of at least one neurodegenerative disorder and/or dementia, wherein the medicament comprises from about 1 mg to about 1500 mg CBD or a pharmaceutically acceptable salt or derivative thereof per dose.

In another aspect of the present disclosure, there is provided a kit comprising CBD or a pharmaceutically acceptable salt or derivative thereof, and an acetylcholinesterase inhibitor for use in the treatment or prevention of at least one neurodegenerative disorder and/or dementia, wherein the kit comprises the CBD or a pharmaceutically acceptable salt or derivative thereof in a dosage form comprising from about 1 mg to about 1500 mg CBD or a pharmaceutically acceptable salt or derivative thereof per dose.

In another aspect of the present disclosure, there is provided a pharmaceutical composition comprising CBD or a pharmaceutically acceptable salt thereof, and an acetylcholinesterase inhibitor, wherein the pharmaceutical composition comprises from about 1 mg to about 1500 mg CBD or a pharmaceutically acceptable salt or derivative thereof per dose.

### DETAILED DESCRIPTION

Unless defined otherwise, all technical and scientific terms used herein have the same meaning as commonly understood by those of ordinary skill in the art to which the disclosure belongs. Any materials and method similar or equivalent to those described herein can be used to practice the present invention.

Throughout this specification, unless the context requires otherwise, the word “comprise”, or variations such as “comprises” or “comprising”, will be understood to imply the inclusion of the stated element or integer or group of elements or integers but not the exclusion of any other element or integer or group of elements or integers.

The phrase “consisting of” means including, and limited to, whatever follows the phrase “consisting of”. Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a compound” includes a single compound, as well as two or more compounds; reference to “an agent” includes one agent, as well as two or more agents; and so forth.

The term “about” will be understood by persons skilled in the art and will vary to some extent depending on the context in which it is used. If there are uses of the term that are not clear to persons skilled in the art, given the context which it is used, “about” will mean up to plus or minus 10% of the particular term.

In an aspect disclosed herein, there is provided a method for the treatment or prevention of at least one neurodegenerative disorder and/or dementia, the method comprising administering to a subject in need thereof: (i) cannabidiol (CBD) or a pharmaceutically acceptable salt or derivative thereof, and (ii) an acetylcholinesterase inhibitor, wherein the subject is administered from about 1 mg to about 1500 mg CBD or a pharmaceutically acceptable salt or derivative thereof per day.

### Neurodegenerative Disorders

The term “neurodegenerative disorder” as used herein refers to diseases or disorders that are characterized by a progressive decline of the motor and/or cognitive function caused by the selective loss of neurons within the central nervous system, which is associated with deposition of proteins showing altered physiochemical properties in the brain and in peripheral organs.

Neurodegenerative disorders can be classified by primary clinical features (e.g., dementia, parkinsonisms, or motor neuron disease), anatomic distribution of neurodegeneration (e.g., frontotemporal degenerations, extrapyramidal disorders, or spinocerebellar degenerations) or molecular features (e.g., amyloidoses, tauopathies, α-synucleinopathies, and TDP-43 proteinopathies, proteotoxic stress and its attendant abnormalities in ubiquitin-proteasomal and autophagosomal/lysosomal systems, oxidative stress, programmed cell death, and neuroinflammation). Neurogenerative disorders can also be staged using clinical instruments, such as Braak staging. For instance, Braak stages I and TT is often associated with normal cognitive and clinical profiles, with some tau-based neurofibrillary tangle (NFT) pathology. Braak stage III and IV are referred to as the limbic phase, with increased NFT pathology and limited cognitive dysfunction observed. Braak stages V and VI are referred to as the neocortical stages, which presented with the greatest number and extent of NFTs and significant cognitive impairment.

In an embodiment, the neurodegenerative disorder is mild cognitive impairment.

In an embodiment, the neurodegenerative disorder is Braak stage I, II, III, IV, V or VI.

In an embodiment, the neurodegenerative disorder is selected from the group consisting of Alzheimer's disease, Parkinson's disease, Huntington's disease, chronic traumatic encephalopathy (CTE), amyotrophic lateral sclerosis (ALS), neurodegeneration with brain iron accumulation, mitochondrial dysfunction, Huntington's disease, ataxia, and neuronal ceroid lipofuscinosis (NCL).

In an embodiment, the neurodegenerative disorder is Alzheimer's disease.

In an embodiment, the neurodegenerative disorder is selected from the group consisting of Parkinson's disease, Alzheimer's disease, Huntington's disease and ALS.

### Dementia

The terms “dementia”, “major neurocognitive disorder”, or “MND” may be used interchangeably herein refer to a syndrome associated with a range of diseases (e.g., Alzheimer's disease, vascular dementia, Parkinson's disease, dementia with Lewy bodies, frontotemporal dementia, Huntington's disease, alcohol-related dementia, Downs syndrome, human immunodeficiency virus (HIV), and chronic traumatic encephalopathy (CTE)), which is characterized by the impairment of brain functions, including language, memory, perception, personality and cognitive skills. In the early stages of dementia, difficulty may be faced with familiar tasks such as shopping, driving or handling money. As dementia progresses, more basic or core activities of daily living, such as self-care, are affected. The cognitive, psychiatric and behavioral manifestations of dementia may include memory problems, communication difficulties (e.g., problems with speech and understanding language), confusion, wandering, getting lost, personality changes, depression, delusions, apathy and withdrawal.

Dementia can be classified using the Diagnostic and Statistical Manual of Mental Disorders (DSM; American Psychiatric Association, 2013, Diagnostic and statistical manual of mental disorders. 5^{th} ed. Arlington, VA: APA), which is based on the consideration of six cognitive domains: perceptual-motor function (e.g., visual perception, visuoconstructional reasoning, perceptual-motor coordination), language (e.g., object naming, word finding, fluency, grammar and syntax, receptive language), learning and memory (e.g., free recall, cued recall, recognition memory, semantic and autobiographical long-term memory, implicit learning), social cognition (e.g., recognition of emotions, theory of mind, insight), complex attention (e.g., sustained attention, divided attention, selective attention, processing speed), and executive function (e.g., planning, decision-making, working memory, responding to feedback, inhibition, flexibility). Methods for the measurement of cognitive domains would be known to persons skilled in the art, illustrative examples of which include neurophysiological testing and clinical assessment.

In an embodiment, the dementia is selected from the group consisting of mild dementia, moderate dementia and severe dementia.

In certain embodiments, the severity of dementia is characterized with reference to a subjective cognitive score, e.g., the Mini Mental State Examination (MMSE) score (Folstein et al., 1975, Journal of Psychiatric Research, 12(3): 189-98). According to such embodiments, mild dementia is characterized by an MMSE score of from about 21 to about 26, moderate dementia is characterized by an MMSE score of from about 10 to about 20, and advanced dementia is characterized by an MMSE score of <10.

In an embodiment, the dementia is mild to moderate dementia. In another embodiment, the dementia is moderate to severe dementia.

The classification of dementia may also be made in consideration of biomarkers of dementia, such as imaging modalities (e.g., structural brain imaging, functional imaging) and fluid biomarkers (e.g., cerebrospinal fluid, blood and urine levels of, e.g., amyloid-beta, tau, phosphorylated tau, inflammatory markers, matrix metalloproteinases, angiogenic markers).

In an embodiment, the dementia is selected from the group consisting of vascular dementia, dementia with Lewy bodies, frontotemporal dementia, HIV-induced dementia, and traumatic brain injury (TBI) with dementia.

In an embodiment, the subject with dementia has at least one neurodegenerative disorder selected from the group consisting of Alzheimer's disease, Parkinson's disease, Huntington's disease, chronic traumatic encephalopathy (CTE), amyotrophic lateral sclerosis (ALS), neurodegeneration with brain iron accumulation, mitochondrial dysfunction, Huntington's disease, ataxia, and neuronal ceroid lipofuscinosis (NCL).

In an embodiment, the at least one neurodegenerative disorder is selected from the group consisting of Parkinson's disease, Alzheimer's disease, Huntington's disease and ALS.

In an embodiment, the at least one neurodegenerative disorder is Alzheimer's disease.

Diseases Associated with Glutamate-Mediated Excitotoxicity

The term “diseases associated with glutamate-mediated excitotoxicity” as used herein refers to diseases or disorders that are associated with excessive exposure to glutamate, or overstimulation of its membrane receptors, which leads to loss of neuronal function, neuronal injury or death. Excitotoxicity can be triggered by, for example, physical trauma, oxidative stress, oxygen deprivation, mitochondrial toxins, and changes in central nervous system pH levels.

Glutamate-mediated excitotoxicity is associated with many disease states, including ischemia, traumatic brain injury (TBI), epilepsy, hypoglycemia and status epilepticus (see, e.g., Meldrum and Garthwaite, 1990, Trends in Pharmacological Sciences, 11: 379-387), and neurodegenerative disorders such as Parkinson's disease, Alzheimer's disease, Huntington's disease and amyotrophic lateral sclerosis (ALS) (see, e.g., Dong et al., 2009, Acta Pharmacologica Sinica, 30(4): 379-387; Lewerenz and Maher, 2015, Frontiers in Neuroscience, 9, Article 469: 1-20).

In an embodiment, the disease associated with glutamate-mediated excitotoxicity is an acute disease selected from the group consisting of ischemia, TBI, epilepsy, hypoglycemia, and status epilepticus.

In an embodiment, the disease associated with glutamate-mediated excitotoxicity is a chronic neurodegenerative disorder selected from the group consisting of Parkinson's disease, Alzheimer's disease, Huntington's disease and amyotrophic lateral sclerosis (ALS).

### Cannabidiol (CBD)

The terms “cannabidiol” or “CBD” are used interchangeably herein to refer to a cannabinoid produced by plants of the genus Cannabis. CBD has antagonist activity on agonists of the CB1 and CB2 receptors and acts as an inverse agonist of the CB1 and CB2 receptors.

CBD is synthesized in Cannabis plants as cannabidiolic acid (CBDA), which decarboxylates to CBD. While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying and/or heating the plant material. Persons skilled in the art would be familiar with methods by which decarboxylation of CBDA can be promoted, illustrative examples of which include air-drying, combustion, vaporization, curing, heating and baking. The decarboxylated CBD will typically bind to and/or stimulate, directly or indirectly, cannabinoid receptors including CB1 and/or CB2.

CBD may be extracted from any suitable plant parts including leaves, flowers or stems and may be produced by any suitable means known to those skilled in the art. For example, CBD extracts may be produced by extraction with supercritical or subcritical CO₂, or by volatilization of plant material with a heated gas. Illustrative examples of methods used the extract CBD and other cannabinoids from plant material include the methods described in U.S. patent Ser. No. 10/189,762 and WO 2004/016277.

In an embodiment, the CBD is naturally-derived CBD.

In an embodiment, the CBD is synthetic CBD (i.e., non-naturally-derived CBD).

CBD is a chiral compound, although only the (−) CBD enantiomer is present in Cannabis plants.

The term “enantiomer” as used herein refers to asymmetric molecules that can exist in two different isomeric forms, which have different configurations in space. An enantiomer can rotate plane-polarized light and is, therefore, optically active. Two different enantiomers of the same compound will rotate plane-polarized light in the opposite direction, thus the light can be rotated to the left or counterclockwise for a hypothetical observer (i.e., “levorotatory” or “−”) or it can be rotated to the right or clockwise (i.e., “dextrorotatory” or

In an embodiment, the synthetic CBD is a racemic mixture, comprising the (−) CBD enantiomer and the (+) CBD enantiomer.

In an embodiment, the synthetic CBD consists of the (−) CBD enantiomer.

The present disclosure contemplates the use of pharmaceutically acceptable salts of CBD. Suitable pharmaceutically acceptable salts of CBD would be known to persons skilled in the art, illustrative examples of which include salts or esters prepared from pharmaceutically acceptable non-toxic bases or acids, including inorganic bases or acids and organic bases or acids, which would be known to persons skilled in the art.

The present disclosure further contemplates the use of functional derivatives of CBD. Suitable functional derivatives of CBD would be known to persons skilled in the art, illustrative examples of which include 7-OH-CBD (7-hydroxycannabidiol), methoxylated CBD derivatives (e.g., CBDM, or 2-methoxycannabidiol and CBDD, or 2,6-dimethoxycannabidiol), cannabidiorcol (CBD-Ci) and the CBD derivatives described by Morales et al. (2017, Frontiers in Pharmacology, 8: 422).

Illustrative examples of the structures of CBD or pharmaceutically acceptable salts or derivatives thereof are shown in Table 1.

### Acetylcholinesterase Inhibitors

As used herein, the term “acetylcholinesterase inhibitor” refers to a class of agents that inhibit the activity of acetylcholinesterase, an enzyme that is responsible for catalyzing the reaction between acetylcholine and water into choline and acetate. The suppression of acetylcholinesterase leads to the increase of both the level and duration of action of acetylcholine within the synapses, leading to increased cholinergic signaling within the central nervous system, autonomic ganglia and neuromuscular junctions. Acetylcholinesterase inhibitors are used in the treatment of neurodegenerative diseases (e.g., Alzheimer's disease and Parkinson's disease), myasthenia gravis, anticholinergic poisoning, glaucoma, and psychiatric disorders (e.g., schizophrenia). Suitable acetylcholinesterase inhibitors will be known to persons skilled in the art, illustrative examples of which include donepezil, rivastigmine, galantamine, neostigmine, and physostigmine. In an embodiment, the acetylcholinesterase inhibitor is a non-naturally occurring molecule, e.g., man-made, synthetic, etc.

In an embodiment, the acetylcholinesterase inhibitor is selected from the group consisting of donepezil, rivastigmine and galantamine.

“Donepezil” is a reversible acetylcholinesterase inhibitor with the structure of formula (VI).

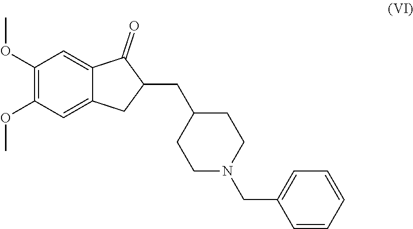 [SMILES: COc1cc2c(cc1OC)C(=O)C(CC1CCN(Cc3ccccc3)CC1)C2]

“Rivastigmine” is a reversible acetylcholinesterase and butyrylcholinesterase inhibitor with the structure of formula (VII).

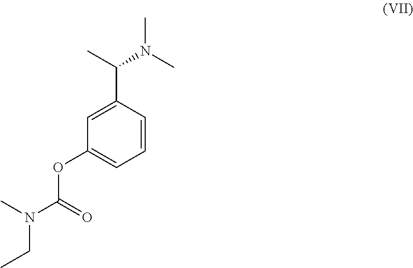

“Galantamine” is reversible acetylcholinesterase inhibitor with the structure of formula (VIII).

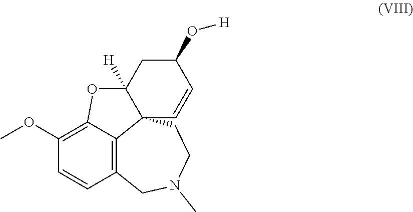 [SMILES: COc1ccc2c3c1O[C@H]1C[C@@H](O)C=C[C@@]31CCN(C)C2]

In an embodiment, the acetylcholinesterase inhibitor is donepezil, or a pharmaceutically acceptable salt thereof.

Pharmaceutically acceptable salts of donepezil would be known to persons skilled in the art, illustrative examples of which include donepezil hydrochloride, donepezil oxalate, donepezil mesylate, donepezil besylate and donepezil tosylate.

In an embodiment, the pharmaceutically acceptable salt of donepezil is donepezil hydrochloride.

Methods for the Treatment or Prevention of a Neurodegenerative Disorder and/or Dementia

As disclosed elsewhere herein, the present disclosure provides a method for the treatment or prevention of at least one neurodegenerative disorder and/or dementia, the method comprising administering to a subject in need thereof: (i) cannabidiol (CBD) or a pharmaceutically acceptable salt or derivative thereof, and (ii) an acetylcholinesterase inhibitor, wherein the subject is administered from about 1 mg to about 1500 mg CBD or a pharmaceutically acceptable salt or derivative thereof per day.

In another aspect disclosed herein, there is provided a method for the treatment or prevention of at least one neurodegenerative disorder, the method comprising administering to a subject in need thereof: (i) cannabidiol (CBD) or a pharmaceutically acceptable salt or derivative thereof, and (ii) an acetylcholinesterase inhibitor, wherein the subject is administered from about 1 mg to about 1500 mg CBD or a pharmaceutically acceptable salt or derivative thereof per day.

In an aspect disclosed herein, there is provided a method for the treatment or prevention of dementia, the method comprising administering to a subject in need thereof: (i) cannabidiol (CBD) or a pharmaceutically acceptable salt or derivative thereof, and (ii) an acetylcholinesterase inhibitor, wherein the subject is administered from about 1 mg to about 1500 mg CBD or a pharmaceutically acceptable salt or derivative thereof per day.

The terms “treat”, “treating”, “treatment” and the like are used interchangeably herein to mean relieving, reducing, alleviating, ameliorating or otherwise inhibiting the severity of one or more symptoms of dementia in a subject. The terms “prevent”, “preventing”, “prophylaxis”, “prophylactic”, “preventative” and the like are used interchangeably herein to mean preventing or delaying the onset of at least one neurodegenerative disorder and/or dementia, or the risk of developing at least one neurodegenerative disorder and/or dementia.

It is to be understood that the terms “treat”, “treating”, “treatment” and the like, as used herein, do not imply that a subject is treated until the at least one neurodegenerative disorder and/or dementia has been eliminated or are no longer evident. Said treatment may also reduce the severity of the one or more symptoms of the at least one neurodegenerative disorder and/or dementia.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans for whom treatment or prophylaxis of at least one neurodegenerative disorder and/or dementia is desired. In an embodiment, the subject is a human.

It is to be understood that the CBD or a pharmaceutically acceptable salt or derivative thereof, and the acetylcholinesterase inhibitor will be administered to the subject in need thereof in a therapeutically effective amount. As used herein, the term “therapeutically effective amount” typically refers to an amount of the CBD and an amount of the acetylcholinesterase inhibitor that is sufficient to affect one or more beneficial or desired therapeutic outcomes (e.g., reduction in the decline in functional capacities, symptomatic treatment of cognitive function, decreased levels of inflammatory markers, e.g., TNF-α, IL-1β, IL-6, Cox-2, and nitric oxide, reduction in amyloid-beta plaques/aggregation, reduction in tau phosphorylation, reduction in oxidative stress, improved mitochondrial function, improvement in cognitive function, mood and well-being, and reduction in glutamate toxicity). Said beneficial or desired therapeutic outcomes may be quantified by using clinical instruments known in the art, illustrative examples of which include the MMSE, the Abbreviated Mental Test Score (AMTS; Qureshi and Hodkinson, 1974, Age Ageing, 3: 152-157), clock-drawing test (Broday and Moore, 1997, Geriatric Psychiatry, 12: 619-627), Mini-Cog (Borson et al., 2000, Geriatric Psychiatry, 15: 1021-1027), 6-CIT (Brooke and Bullock, 1999, International Journal of Geriatric Psychiatry, 14: 936-940), General Practitioner Assessment of Cognition (GPCOG; Brodaty et al., 2002, Journal of the American Geriatrics Society, 50: 530-534), the Neuropsychiatric Inventory (Cummings et al., 1994, Neurology, 31: 2308-2314), the EuroQol measure (EuroQol Group, 1990, Health Policy, 16: 199-208), Alzheimer's Disease-related Quality of Life Scale (QoL-AD; Logsdon et al., 1999, Journal of Mental Health and Aging, 5: 21-32), DEMQOL (Smith et al., 2007, Psychological Medicine, 37: 737-746), Geriatric Depression Scale (GDS; Yesavage et al., 1983, Journal of Psychiatric Research, 17: 37-49), and the like.

Changes in the symptoms or severity of at least one neurodegenerative disorder and/or dementia as measured by any of the quantitative methods or clinical instruments described elsewhere herein may be expressed using any appropriate statistical measure to demonstrate the magnitude of the reduction in the symptoms or severity of the at least one neurodegenerative disorder and/or dementia. In an embodiment, the methods disclosed herein reduce in the symptoms or severity of the at least one neurodegenerative disorder and/or dementia by at least 10%, preferably at least 20%, preferably at least 30%, preferably at least 40%, preferably at least 50%, preferably at least 60%, preferably at least 70%, preferably at least 80%, preferably at least 90%, or more preferably at least 100% as compared to a subject with the same neurodegenerative disorder(s) and/or dementia who has not been administered CBD or a pharmaceutically acceptable salt or derivative thereof, and an acetylcholinesterase inhibitor.

In an embodiment, the methods disclosed herein slow the progression of the at least one neurodegenerative disorder and/or dementia.

In an embodiment, the methods disclosed herein slow the progression of the at least one neurodegenerative disorder and/or dementia by >10% as compared to a subject with the same neurodegenerative disorder(s) and/or dementia who has not been administered CBD or a pharmaceutically acceptable salt or derivative thereof, and an acetylcholinesterase inhibitor.

In an embodiment, the methods disclosed herein prevent the development of dementia.

In an embodiment, the subject is administered from about 1 mg to about 50 mg of the acetylcholinesterase inhibitor per day (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg of the acetylcholinesterase inhibitor per day).

Thus, in an embodiment the subject is administered from about 1 mg to about 50 mg of the acetylcholinesterase inhibitor per day, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, or more preferably about 50 mg of the acetylcholinesterase inhibitor per day.

In an embodiment, the subject is administered from about 5 mg to about 10 mg of the acetylcholinesterase inhibitor per day.

In an embodiment, the subject is administered from about 10 mg to about 25 mg of the acetylcholinesterase inhibitor per day.

In an embodiment, the acetylcholinesterase inhibitor is donepezil or a pharmaceutically acceptable salt thereof, and wherein the subject is administered from about 5 mg to about 10 mg of the donepezil or a pharmaceutically acceptable salt thereof per day.

In an embodiment, the acetylcholinesterase inhibitor is donepezil or a pharmaceutically acceptable salt thereof, and wherein the subject is administered from about 10 mg to about 25 mg of the donepezil or a pharmaceutically acceptable salt thereof per day.

In an embodiment, the acetylcholinesterase inhibitor is donepezil hydrochloride or a pharmaceutically acceptable salt thereof, and wherein the subject is administered from about 5 mg to about 10 mg of the donepezil hydrochloride or a pharmaceutically acceptable salt thereof per day.

In an embodiment, the acetylcholinesterase inhibitor is donepezil hydrochloride or a pharmaceutically acceptable salt thereof, and wherein the subject is administered from about 10 mg to about 25 mg of the donepezil hydrochloride or a pharmaceutically acceptable salt thereof per day.

In an embodiment, the acetylcholinesterase inhibitor is rivastigmine or a pharmaceutically acceptable salt thereof, and wherein the subject is administered from about 1 mg to about 20 mg of the rivastigmine or a pharmaceutically acceptable salt thereof per day.

In an embodiment, the acetylcholinesterase inhibitor is rivastigmine or a pharmaceutically acceptable salt thereof, and wherein the subject is administered from about 3 mg to about 12 mg of the rivastigmine or a pharmaceutically acceptable salt thereof per day.

In an embodiment, the acetylcholinesterase inhibitor is galantamine or a pharmaceutically acceptable salt thereof, and wherein the subject is administered from about 5 mg to about 50 mg of the galantamine or a pharmaceutically acceptable salt thereof per day.

In an embodiment, the acetylcholinesterase inhibitor is galantamine or a pharmaceutically acceptable salt thereof, and wherein the subject is administered from about 8 mg to about 24 mg of the galantamine or a pharmaceutically acceptable salt thereof per day.

In an embodiment, the subject is administered from about 1 mg to about 1500 mg CBD or a pharmaceutically acceptable salt or derivative thereof per day (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, 1000 mg, 1001 mg, 1002 mg, 1003 mg, 1004 mg, 1005 mg, 1006 mg, 1007 mg, 1008 mg, 1009 mg, 1010 mg, 1011 mg, 1012 mg, 1013 mg, 1014 mg, 1015 mg, 1016 mg, 1017 mg, 1018 mg, 1019 mg, 1020 mg, 1021 mg, 1022 mg, 1023 mg, 1024 mg, 1025 mg, 1026 mg, 1027 mg, 1028 mg, 1029 mg, 1030 mg, 1031 mg, 1032 mg, 1033 mg, 1034 mg, 1035 mg, 1036 mg, 1037 mg, 1038 mg, 1039 mg, 1040 mg, 1041 mg, 1042 mg, 1043 mg, 1044 mg, 1045 mg, 1046 mg, 1047 mg, 1048 mg, 1049 mg, 1050 mg, 1051 mg, 1052 mg, 1053 mg, 1054 mg, 1055 mg, 1056 mg, 1057 mg, 1058 mg, 1059 mg, 1060 mg, 1061 mg, 1062 mg, 1063 mg, 1064 mg, 1065 mg, 1066 mg, 1067 mg, 1068 mg, 1069 mg, 1070 mg, 1071 mg, 1072 mg, 1073 mg, 1074 mg, 1075 mg, 1076 mg, 1077 mg, 1078 mg, 1079 mg, 1080 mg, 1081 mg, 1082 mg, 1083 mg, 1084 mg, 1085 mg, 1086 mg, 1087 mg, 1088 mg, 1089 mg, 1090 mg, 1091 mg, 1092 mg, 1093 mg, 1094 mg, 1095 mg, 1096 mg, 1097 mg, 1098 mg, 1099 mg, 1100 mg, 1101 mg, 1102 mg, 1103 mg, 1104 mg, 1105 mg, 1106 mg, 1107 mg, 1108 mg, 1109 mg, 1110 mg, 1111 mg, 1112 mg, 1113 mg, 1114 mg, 1115 mg, 1116 mg, 1117 mg, 1118 mg, 1119 mg, 1120 mg, 1121 mg, 1122 mg, 1123 mg, 1124 mg, 1125 mg, 1126 mg, 1127 mg, 1128 mg, 1129 mg, 1130 mg, 1131 mg, 1132 mg, 1133 mg, 1134 mg, 1135 mg, 1136 mg, 1137 mg, 1138 mg, 1139 mg, 1140 mg, 1141 mg, 1142 mg, 1143 mg, 1144 mg, 1145 mg, 1146 mg, 1147 mg, 1148 mg, 1149 mg, 1150 mg, 1151 mg, 1152 mg, 1153 mg, 1154 mg, 1155 mg, 1156 mg, 1157 mg, 1158 mg, 1159 mg, 1160 mg, 1161 mg, 1162 mg, 1163 mg, 1164 mg, 1165 mg, 1166 mg, 1167 mg, 1168 mg, 1169 mg, 1170 mg, 1171 mg, 1172 mg, 1173 mg, 1174 mg, 1175 mg, 1176 mg, 1177 mg, 1178 mg, 1179 mg, 1180 mg, 1181 mg, 1182 mg, 1183 mg, 1184 mg, 1185 mg, 1186 mg, 1187 mg, 1188 mg, 1189 mg, 1190 mg, 1191 mg, 1192 mg, 1193 mg, 1194 mg, 1195 mg, 1196 mg, 1197 mg, 1198 mg, 1199 mg, 1200 mg, 1201 mg, 1202 mg, 1203 mg, 1204 mg, 1205 mg, 1206 mg, 1207 mg, 1208 mg, 1209 mg, 1210 mg, 1211 mg, 1212 mg, 1213 mg, 1214 mg, 1215 mg, 1216 mg, 1217 mg, 1218 mg, 1219 mg, 1220 mg, 1221 mg, 1222 mg, 1223 mg, 1224 mg, 1225 mg, 1226 mg, 1227 mg, 1228 mg, 1229 mg, 1230 mg, 1231 mg, 1232 mg, 1233 mg, 1234 mg, 1235 mg, 1236 mg, 1237 mg, 1238 mg, 1239 mg, 1240 mg, 1241 mg, 1242 mg, 1243 mg, 1244 mg, 1245 mg, 1246 mg, 1247 mg, 1248 mg, 1249 mg, 1250 mg, 1251 mg, 1252 mg, 1253 mg, 1254 mg, 1255 mg, 1256 mg, 1257 mg, 1258 mg, 1259 mg, 1260 mg, 1261 mg, 1262 mg, 1263 mg, 1264 mg, 1265 mg, 1266 mg, 1267 mg, 1268 mg, 1269 mg, 1270 mg, 1271 mg, 1272 mg, 1273 mg, 1274 mg, 1275 mg, 1276 mg, 1277 mg, 1278 mg, 1279 mg, 1280 mg, 1281 mg, 1282 mg, 1283 mg, 1284 mg, 1285 mg, 1286 mg, 1287 mg, 1288 mg, 1289 mg, 1290 mg, 1291 mg, 1292 mg, 1293 mg, 1294 mg, 1295 mg, 1296 mg, 1297 mg, 1298 mg, 1299 mg, 1300 mg, 1301 mg, 1302 mg, 1303 mg, 1304 mg, 1305 mg, 1306 mg, 1307 mg, 1308 mg, 1309 mg, 1310 mg, 1311 mg, 1312 mg, 1313 mg, 1314 mg, 1315 mg, 1316 mg, 1317 mg, 1318 mg, 1319 mg, 1320 mg, 1321 mg, 1322 mg, 1323 mg, 1324 mg, 1325 mg, 1326 mg, 1327 mg, 1328 mg, 1329 mg, 1330 mg, 1331 mg, 1332 mg, 1333 mg, 1334 mg, 1335 mg, 1336 mg, 1337 mg, 1338 mg, 1339 mg, 1340 mg, 1341 mg, 1342 mg, 1343 mg, 1344 mg, 1345 mg, 1346 mg, 1347 mg, 1348 mg, 1349 mg, 1350 mg, 1351 mg, 1352 mg, 1353 mg, 1354 mg, 1355 mg, 1356 mg, 1357 mg, 1358 mg, 1359 mg, 1360 mg, 1361 mg, 1362 mg, 1363 mg, 1364 mg, 1365 mg, 1366 mg, 1367 mg, 1368 mg, 1369 mg, 1370 mg, 1371 mg, 1372 mg, 1373 mg, 1374 mg, 1375 mg, 1376 mg, 1377 mg, 1378 mg, 1379 mg, 1380 mg, 1381 mg, 1382 mg, 1383 mg, 1384 mg, 1385 mg, 1386 mg, 1387 mg, 1388 mg, 1389 mg, 1390 mg, 1391 mg, 1392 mg, 1393 mg, 1394 mg, 1395 mg, 1396 mg, 1397 mg, 1398 mg, 1399 mg, 1400 mg, 1401 mg, 1402 mg, 1403 mg, 1404 mg, 1405 mg, 1406 mg, 1407 mg, 1408 mg, 1409 mg, 1410 mg, 1411 mg, 1412 mg, 1413 mg, 1414 mg, 1415 mg, 1416 mg, 1417 mg, 1418 mg, 1419 mg, 1420 mg, 1421 mg, 1422 mg, 1423 mg, 1424 mg, 1425 mg, 1426 mg, 1427 mg, 1428 mg, 1429 mg, 1430 mg, 1431 mg, 1432 mg, 1433 mg, 1434 mg, 1435 mg, 1436 mg, 1437 mg, 1438 mg, 1439 mg, 1440 mg, 1441 mg, 1442 mg, 1443 mg, 1444 mg, 1445 mg, 1446 mg, 1447 mg, 1448 mg, 1449 mg, 1450 mg, 1451 mg, 1452 mg, 1453 mg, 1454 mg, 1455 mg, 1456 mg, 1457 mg, 1458 mg, 1459 mg, 1460 mg, 1461 mg, 1462 mg, 1463 mg, 1464 mg, 1465 mg, 1466 mg, 1467 mg, 1468 mg, 1469 mg, 1470 mg, 1471 mg, 1472 mg, 1473 mg, 1474 mg, 1475 mg, 1476 mg, 1477 mg, 1478 mg, 1479 mg, 1480 mg, 1481 mg, 1482 mg, 1483 mg, 1484 mg, 1485 mg, 1486 mg, 1487 mg, 1488 mg, 1489 mg, 1490 mg, 1491 mg, 1492 mg, 1493 mg, 1494 mg, 1495 mg, 1496 mg, 1497 mg, 1498 mg, 1499 mg, or 1500 mg CBD or a pharmaceutically acceptable salt or derivative thereof per day).

Thus, in an embodiment, the subject is administered from about 1 mg to about 1500 mg CBD or a pharmaceutically acceptable salt or derivative thereof per day, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, preferably about 100 mg, preferably about 101 mg, preferably about 102 mg, preferably about 103 mg, preferably about 104 mg, preferably about 105 mg, preferably about 106 mg, preferably about 107 mg, preferably about 108 mg, preferably about 109 mg, preferably about 110 mg, preferably about 111 mg, preferably about 112 mg, preferably about 113 mg, preferably about 114 mg, preferably about 115 mg, preferably about 116 mg, preferably about 117 mg, preferably about 118 mg, preferably about 119 mg, preferably about 120 mg, preferably about 121 mg, preferably about 122 mg, preferably about 123 mg, preferably about 124 mg, preferably about 125 mg, preferably about 126 mg, preferably about 127 mg, preferably about 128 mg, preferably about 129 mg, preferably about 130 mg, preferably about 131 mg, preferably about 132 mg, preferably about 131 mg, preferably about 134 mg, preferably about 135 mg, preferably about 136 mg, preferably about 137 mg, preferably about 138 mg, preferably about 139 mg, preferably about 140 mg, preferably about 141 mg, preferably about 142 mg, preferably about 143 mg, preferably about 144 mg, preferably about 145 mg, preferably about 146 mg, preferably about 147 mg, preferably about 148 mg, preferably about 149 mg, preferably about 150 mg, preferably about 151 mg, preferably about 152 mg, preferably about 153 mg, preferably about 154 mg, preferably about 155 mg, preferably about 156 mg, preferably about 157 mg, preferably about 158 mg, preferably about 159 mg, preferably about 160 mg, preferably about 161 mg, preferably about 162 mg, preferably about 163 mg, preferably about 164 mg, preferably about 165 mg, preferably about 166 mg, preferably about 167 mg, preferably about 168 mg, preferably about 169 mg, preferably about 170 mg, preferably about 171 mg, preferably about 172 mg, preferably about 173 mg, preferably about 174 mg, preferably about 175 mg, preferably about 176 mg, preferably about 177 mg, preferably about 178 mg, preferably about 179 mg, preferably about 180 mg, preferably about 181 mg, preferably about 182 mg, preferably about 183 mg, preferably about 184 mg, preferably about 185 mg, preferably about 186 mg, preferably about 187 mg, preferably about 188 mg, preferably about 189 mg, preferably about 190 mg, preferably about 191 mg, preferably about 192 mg, preferably about 193 mg, preferably about 194 mg, preferably about 195 mg, preferably about 196 mg, preferably about 197 mg, preferably about 198 mg, preferably about 199 mg, preferably about 200 mg, preferably about 201 mg, preferably about 202 mg, preferably about 203 mg, preferably about 204 mg, preferably about 205 mg, preferably about 206 mg, preferably about 207 mg, preferably about 208 mg, preferably about 209 mg, preferably about 210 mg, preferably about 211 mg, preferably about 212 mg, preferably about 213 mg, preferably about 214 mg, preferably about 215 mg, preferably about 216 mg, preferably about 217 mg, preferably about 218 mg, preferably about 219 mg, preferably about 220 mg, preferably about 221 mg, preferably about 222 mg, preferably about 223 mg, preferably about 224 mg, preferably about 225 mg, preferably about 226 mg, preferably about 227 mg, preferably about 228 mg, preferably about 229 mg, preferably about 230 mg, preferably about 231 mg, preferably about 232 mg, preferably about 233 mg, preferably about 234 mg, preferably about 235 mg, preferably about 236 mg, preferably about 237 mg, preferably about 238 mg, preferably about 239 mg, preferably about 240 mg, preferably about 241 mg, preferably about 242 mg, preferably about 243 mg, preferably about 244 mg, preferably about 245 mg, preferably about 246 mg, preferably about 247 mg, preferably about 248 mg, preferably about 249 mg, preferably about 250 mg, preferably about 251 mg, preferably about 252 mg, preferably about 253 mg, preferably about 254 mg, preferably about 255 mg, preferably about 256 mg, preferably about 257 mg, preferably about 258 mg, preferably about 259 mg, preferably about 260 mg, preferably about 261 mg, preferably about 262 mg, preferably about 263 mg, preferably about 264 mg, preferably about 265 mg, preferably about 266 mg, preferably about 267 mg, preferably about 268 mg, preferably about 269 mg, preferably about 270 mg, preferably about 271 mg, preferably about 272 mg, preferably about 273 mg, preferably about 274 mg, preferably about 275 mg, preferably about 276 mg, preferably about 277 mg, preferably about 278 mg, preferably about 279 mg, preferably about 280 mg, preferably about 281 mg, preferably about 282 mg, preferably about 283 mg, preferably about 284 mg, preferably about 285 mg, preferably about 286 mg, preferably about 287 mg, preferably about 288 mg, preferably about 289 mg, preferably about 290 mg, preferably about 291 mg, preferably about 292 mg, preferably about 293 mg, preferably about 294 mg, preferably about 295 mg, preferably about 296 mg, preferably about 297 mg, preferably about 298 mg, preferably about 299 mg, preferably about 300 mg, preferably about 301 mg, preferably about 302 mg, preferably about 303 mg, preferably about 304 mg, preferably about 305 mg, preferably about 306 mg, preferably about 307 mg, preferably about 308 mg, preferably about 309 mg, preferably about 310 mg, preferably about 311 mg, preferably about 312 mg, preferably about 313 mg, preferably about 314 mg, preferably about 315 mg, preferably about 316 mg, preferably about 317 mg, preferably about 318 mg, preferably about 319 mg, preferably about 320 mg, preferably about 321 mg, preferably about 322 mg, preferably about 323 mg, preferably about 324 mg, preferably about 325 mg, preferably about 326 mg, preferably about 327 mg, preferably about 328 mg, preferably about 329 mg, preferably about 330 mg, preferably about 331 mg, preferably about 332 mg, preferably about 333 mg, preferably about 334 mg, preferably about 335 mg, preferably about 336 mg, preferably about 337 mg, preferably about 338 mg, preferably about 339 mg, preferably about 340 mg, preferably about 341 mg, preferably about 342 mg, preferably about 343 mg, preferably about 344 mg, preferably about 345 mg, preferably about 346 mg, preferably about 347 mg, preferably about 348 mg, preferably about 349 mg, preferably about 350 mg, preferably about 351 mg, preferably about 352 mg, preferably about 353 mg, preferably about 354 mg, preferably about 355 mg, preferably about 356 mg, preferably about 357 mg, preferably about 358 mg, preferably about 359 mg, preferably about 360 mg, preferably about 361 mg, preferably about 362 mg, preferably about 363 mg, preferably about 364 mg, preferably about 365 mg, preferably about 366 mg, preferably about 367 mg, preferably about 368 mg, preferably about 369 mg, preferably about 370 mg, preferably about 371 mg, preferably about 372 mg, preferably about 373 mg, preferably about 374 mg, preferably about 375 mg, preferably about 376 mg, preferably about 377 mg, preferably about 378 mg, preferably about 379 mg, preferably about 380 mg, preferably about 381 mg, preferably about 382 mg, preferably about 383 mg, preferably about 384 mg, preferably about 385 mg, preferably about 386 mg, preferably about 387 mg, preferably about 388 mg, preferably about 389 mg, preferably about 390 mg, preferably about 391 mg, preferably about 392 mg, preferably about 393 mg, preferably about 394 mg, preferably about 395 mg, preferably about 396 mg, preferably about 397 mg, preferably about 398 mg, preferably about 399 mg, preferably about 400 mg, preferably about 401 mg, preferably about 402 mg, preferably about 403 mg, preferably about 404 mg, preferably about 405 mg, preferably about 406 mg, preferably about 407 mg, preferably about 408 mg, preferably about 409 mg, preferably about 410 mg, preferably about 411 mg, preferably about 412 mg, preferably about 413 mg, preferably about 414 mg, preferably about 415 mg, preferably about 416 mg, preferably about 417 mg, preferably about 418 mg, preferably about 419 mg, preferably about 420 mg, preferably about 421 mg, preferably about 422 mg, preferably about 423 mg, preferably about 424 mg, preferably about 425 mg, preferably about 426 mg, preferably about 427 mg, preferably about 428 mg, preferably about 429 mg, preferably about 430 mg, preferably about 431 mg, preferably about 432 mg, preferably about 433 mg, preferably about 434 mg, preferably about 435 mg, preferably about 436 mg, preferably about 437 mg, preferably about 438 mg, preferably about 439 mg, preferably about 440 mg, preferably about 441 mg, preferably about 442 mg, preferably about 443 mg, preferably about 444 mg, preferably about 445 mg, preferably about 446 mg, preferably about 447 mg, preferably about 448 mg, preferably about 449 mg, preferably about 450 mg, preferably about 451 mg, preferably about 452 mg, preferably about 453 mg, preferably about 454 mg, preferably about 455 mg, preferably about 456 mg, preferably about 457 mg, preferably about 458 mg, preferably about 459 mg, preferably about 460 mg, preferably about 461 mg, preferably about 462 mg, preferably about 463 mg, preferably about 464 mg, preferably about 465 mg, preferably about 466 mg, preferably about 467 mg, preferably about 468 mg, preferably about 469 mg, preferably about 470 mg, preferably about 471 mg, preferably about 472 mg, preferably about 473 mg, preferably about 474 mg, preferably about 475 mg, preferably about 476 mg, preferably about 477 mg, preferably about 478 mg, preferably about 479 mg, preferably about 480 mg, preferably about 481 mg, preferably about 482 mg, preferably about 483 mg, preferably about 484 mg, preferably about 485 mg, preferably about 486 mg, preferably about 487 mg, preferably about 488 mg, preferably about 489 mg, preferably about 490 mg, preferably about 491 mg, preferably about 492 mg, preferably about 493 mg, preferably about 494 mg, preferably about 495 mg, preferably about 496 mg, preferably about 497 mg, preferably about 498 mg, preferably about 499 mg, preferably about 500 mg, preferably about 501 mg, preferably about 502 mg, preferably about 503 mg, preferably about 504 mg, preferably about 505 mg, preferably about 506 mg, preferably about 507 mg, preferably about 508 mg, preferably about 509 mg, preferably about 510 mg, preferably about 511 mg, preferably about 512 mg, preferably about 513 mg, preferably about 514 mg, preferably about 515 mg, preferably about 516 mg, preferably about 517 mg, preferably about 518 mg, preferably about 519 mg, preferably about 520 mg, preferably about 521 mg, preferably about 522 mg, preferably about 523 mg, preferably about 524 mg, preferably about 525 mg, preferably about 526 mg, preferably about 527 mg, preferably about 528 mg, preferably about 529 mg, preferably about 530 mg, preferably about 531 mg, preferably about 532 mg, preferably about 533 mg, preferably about 534 mg, preferably about 535 mg, preferably about 536 mg, preferably about 537 mg, preferably about 538 mg, preferably about 539 mg, preferably about 540 mg, preferably about 541 mg, preferably about 542 mg, preferably about 543 mg, preferably about 544 mg, preferably about 545 mg, preferably about 546 mg, preferably about 547 mg, preferably about 548 mg, preferably about 549 mg, preferably about 550 mg, preferably about 551 mg, preferably about 552 mg, preferably about 553 mg, preferably about 554 mg, preferably about 555 mg, preferably about 556 mg, preferably about 557 mg, preferably about 558 mg, preferably about 559 mg, preferably about 560 mg, preferably about 561 mg, preferably about 562 mg, preferably about 563 mg, preferably about 564 mg, preferably about 565 mg, preferably about 566 mg, preferably about 567 mg, preferably about 568 mg, preferably about 569 mg, preferably about 570 mg, preferably about 571 mg, preferably about 572 mg, preferably about 573 mg, preferably about 574 mg, preferably about 575 mg, preferably about 576 mg, preferably about 577 mg, preferably about 578 mg, preferably about 579 mg, preferably about 580 mg, preferably about 581 mg, preferably about 582 mg, preferably about 583 mg, preferably about 584 mg, preferably about 585 mg, preferably about 586 mg, preferably about 587 mg, preferably about 588 mg, preferably about 589 mg, preferably about 590 mg, preferably about 591 mg, preferably about 592 mg, preferably about 593 mg, preferably about 594 mg, preferably about 595 mg, preferably about 596 mg, preferably about 597 mg, preferably about 598 mg, preferably about 599 mg, preferably about 600 mg, preferably about 601 mg, preferably about 602 mg, preferably about 603 mg, preferably about 604 mg, preferably about 605 mg, preferably about 606 mg, preferably about 607 mg, preferably about 608 mg, preferably about 609 mg, preferably about 610 mg, preferably about 611 mg, preferably about 612 mg, preferably about 613 mg, preferably about 614 mg, preferably about 615 mg, preferably about 616 mg, preferably about 617 mg, preferably about 618 mg, preferably about 619 mg, preferably about 620 mg, preferably about 621 mg, preferably about 622 mg, preferably about 623 mg, preferably about 624 mg, preferably about 625 mg, preferably about 626 mg, preferably about 627 mg, preferably about 628 mg, preferably about 629 mg, preferably about 630 mg, preferably about 631 mg, preferably about 632 mg, preferably about 633 mg, preferably about 634 mg, preferably about 635 mg, preferably about 636 mg, preferably about 637 mg, preferably about 638 mg, preferably about 639 mg, preferably about 640 mg, preferably about 641 mg, preferably about 642 mg, preferably about 643 mg, preferably about 644 mg, preferably about 645 mg, preferably about 646 mg, preferably about 647 mg, preferably about 648 mg, preferably about 649 mg, preferably about 650 mg, preferably about 651 mg, preferably about 652 mg, preferably about 653 mg, preferably about 654 mg, preferably about 655 mg, preferably about 656 mg, preferably about 657 mg, preferably about 658 mg, preferably about 659 mg, preferably about 660 mg, preferably about 661 mg, preferably about 662 mg, preferably about 663 mg, preferably about 664 mg, preferably about 665 mg, preferably about 666 mg, preferably about 667 mg, preferably about 668 mg, preferably about 669 mg, preferably about 670 mg, preferably about 671 mg, preferably about 672 mg, preferably about 673 mg, preferably about 674 mg, preferably about 675 mg, preferably about 676 mg, preferably about 677 mg, preferably about 678 mg, preferably about 679 mg, preferably about 680 mg, preferably about 681 mg, preferably about 682 mg, preferably about 683 mg, preferably about 684 mg, preferably about 685 mg, preferably about 686 mg, preferably about 687 mg, preferably about 688 mg, preferably about 689 mg, preferably about 690 mg, preferably about 691 mg, preferably about 692 mg, preferably about 693 mg, preferably about 694 mg, preferably about 695 mg, preferably about 696 mg, preferably about 697 mg, preferably about 698 mg, preferably about 699 mg, preferably about 700 mg, preferably about 701 mg, preferably about 702 mg, preferably about 703 mg, preferably about 704 mg, preferably about 705 mg, preferably about 706 mg, preferably about 707 mg, preferably about 708 mg, preferably about 709 mg, preferably about 710 mg, preferably about 711 mg, preferably about 712 mg, preferably about 713 mg, preferably about 714 mg, preferably about 715 mg, preferably about 716 mg, preferably about 717 mg, preferably about 718 mg, preferably about 719 mg, preferably about 720 mg, preferably about 721 mg, preferably about 722 mg, preferably about 723 mg, preferably about 724 mg, preferably about 725 mg, preferably about 726 mg, preferably about 727 mg, preferably about 728 mg, preferably about 729 mg, preferably about 730 mg, preferably about 731 mg, preferably about 732 mg, preferably about 733 mg, preferably about 734 mg, preferably about 735 mg, preferably about 736 mg, preferably about 737 mg, preferably about 738 mg, preferably about 739 mg, preferably about 740 mg, preferably about 741 mg, preferably about 742 mg, preferably about 743 mg, preferably about 744 mg, preferably about 745 mg, preferably about 746 mg, preferably about 747 mg, preferably about 748 mg, preferably about 749 mg, preferably about 750 mg, preferably about 751 mg, preferably about 752 mg, preferably about 753 mg, preferably about 754 mg, preferably about 755 mg, preferably about 756 mg, preferably about 757 mg, preferably about 758 mg, preferably about 759 mg, preferably about 760 mg, preferably about 761 mg, preferably about 762 mg, preferably about 763 mg, preferably about 764 mg, preferably about 765 mg, preferably about 766 mg, preferably about 767 mg, preferably about 768 mg, preferably about 769 mg, preferably about 770 mg, preferably about 771 mg, preferably about 772 mg, preferably about 773 mg, preferably about 774 mg, preferably about 775 mg, preferably about 776 mg, preferably about 777 mg, preferably about 778 mg, preferably about 779 mg, preferably about 780 mg, preferably about 781 mg, preferably about 782 mg, preferably about 783 mg, preferably about 784 mg, preferably about 785 mg, preferably about 786 mg, preferably about 787 mg, preferably about 788 mg, preferably about 789 mg, preferably about 790 mg, preferably about 791 mg, preferably about 792 mg, preferably about 793 mg, preferably about 794 mg, preferably about 795 mg, preferably about 796 mg, preferably about 797 mg, preferably about 798 mg, preferably about 799 mg, preferably about 800 mg, preferably about 801 mg, preferably about 802 mg, preferably about 803 mg, preferably about 804 mg, preferably about 805 mg, preferably about 806 mg, preferably about 807 mg, preferably about 808 mg, preferably about 809 mg, preferably about 810 mg, preferably about 811 mg, preferably about 812 mg, preferably about 813 mg, preferably about 814 mg, preferably about 815 mg, preferably about 816 mg, preferably about 817 mg, preferably about 818 mg, preferably about 819 mg, preferably about 820 mg, preferably about 821 mg, preferably about 822 mg, preferably about 823 mg, preferably about 824 mg, preferably about 825 mg, preferably about 826 mg, preferably about 827 mg, preferably about 828 mg, preferably about 829 mg, preferably about 830 mg, preferably about 831 mg, preferably about 832 mg, preferably about 833 mg, preferably about 834 mg, preferably about 835 mg, preferably about 836 mg, preferably about 837 mg, preferably about 838 mg, preferably about 839 mg, preferably about 840 mg, preferably about 841 mg, preferably about 842 mg, preferably about 843 mg, preferably about 844 mg, preferably about 845 mg, preferably about 846 mg, preferably about 847 mg, preferably about 848 mg, preferably about 849 mg, preferably about 850 mg, preferably about 851 mg, preferably about 852 mg, preferably about 853 mg, preferably about 854 mg, preferably about 855 mg, preferably about 856 mg, preferably about 857 mg, preferably about 858 mg, preferably about 859 mg, preferably about 860 mg, preferably about 861 mg, preferably about 862 mg, preferably about 863 mg, preferably about 864 mg, preferably about 865 mg, preferably about 866 mg, preferably about 867 mg, preferably about 868 mg, preferably about 869 mg, preferably about 870 mg, preferably about 871 mg, preferably about 872 mg, preferably about 873 mg, preferably about 874 mg, preferably about 875 mg, preferably about 876 mg, preferably about 877 mg, preferably about 878 mg, preferably about 879 mg, preferably about 880 mg, preferably about 881 mg, preferably about 882 mg, preferably about 883 mg, preferably about 884 mg, preferably about 885 mg, preferably about 886 mg, preferably about 887 mg, preferably about 888 mg, preferably about 889 mg, preferably about 890 mg, preferably about 891 mg, preferably about 892 mg, preferably about 893 mg, preferably about 894 mg, preferably about 895 mg, preferably about 896 mg, preferably about 897 mg, preferably about 898 mg, preferably about 899 mg, preferably about 900 mg, preferably about 901 mg, preferably about 902 mg, preferably about 903 mg, preferably about 904 mg, preferably about 905 mg, preferably about 906 mg, preferably about 907 mg, preferably about 908 mg, preferably about 909 mg, preferably about 910 mg, preferably about 911 mg, preferably about 912 mg, preferably about 913 mg, preferably about 914 mg, preferably about 915 mg, preferably about 916 mg, preferably about 917 mg, preferably about 918 mg, preferably about 919 mg, preferably about 920 mg, preferably about 921 mg, preferably about 922 mg, preferably about 923 mg, preferably about 924 mg, preferably about 925 mg, preferably about 926 mg, preferably about 927 mg, preferably about 928 mg, preferably about 929 mg, preferably about 930 mg, preferably about 931 mg, preferably about 932 mg, preferably about 933 mg, preferably about 934 mg, preferably about 935 mg, preferably about 936 mg, preferably about 937 mg, preferably about 938 mg, preferably about 939 mg, preferably about 940 mg, preferably about 941 mg, preferably about 942 mg, preferably about 943 mg, preferably about 944 mg, preferably about 945 mg, preferably about 946 mg, preferably about 947 mg, preferably about 948 mg, preferably about 949 mg, preferably about 950 mg, preferably about 951 mg, preferably about 952 mg, preferably about 953 mg, preferably about 954 mg, preferably about 955 mg, preferably about 956 mg, preferably about 957 mg, preferably about 958 mg, preferably about 959 mg, preferably about 960 mg, preferably about 961 mg, preferably about 962 mg, preferably about 963 mg, preferably about 964 mg, preferably about 965 mg, preferably about 966 mg, preferably about 967 mg, preferably about 968 mg, preferably about 969 mg, preferably about 970 mg, preferably about 971 mg, preferably about 972 mg, preferably about 973 mg, preferably about 974 mg, preferably about 975 mg, preferably about 976 mg, preferably about 977 mg, preferably about 978 mg, preferably about 979 mg, preferably about 980 mg, preferably about 981 mg, preferably about 982 mg, preferably about 983 mg, preferably about 984 mg, preferably about 985 mg, preferably about 986 mg, preferably about 987 mg, preferably about 988 mg, preferably about 989 mg, preferably about 990 mg, preferably about 991 mg, preferably about 992 mg, preferably about 993 mg, preferably about 994 mg, preferably about 995 mg, preferably about 996 mg, preferably about 997 mg, preferably about 998 mg, preferably about 999 mg, preferably about 1000 mg, preferably about 1001 mg, preferably about 1002 mg, preferably about 1003 mg, preferably about 1004 mg, preferably about 1005 mg, preferably about 1006 mg, preferably about 1007 mg, preferably about 1008 mg, preferably about 1009 mg, preferably about 1010 mg, preferably about 1011 mg, preferably about 1012 mg, preferably about 1013 mg, preferably about 1014 mg, preferably about 1015 mg, preferably about 1016 mg, preferably about 1017 mg, preferably about 1018 mg, preferably about 1019 mg, preferably about 1020 mg, preferably about 1021 mg, preferably about 1022 mg, preferably about 1023 mg, preferably about 1024 mg, preferably about 1025 mg, preferably about 1026 mg, preferably about 1027 mg, preferably about 1028 mg, preferably about 1029 mg, preferably about 1030 mg, preferably about 1031 mg, preferably about 1032 mg, preferably about 1033 mg, preferably about 1034 mg, preferably about 1035 mg, preferably about 1036 mg, preferably about 1037 mg, preferably about 1038 mg, preferably about 1039 mg, preferably about 1040 mg, preferably about 1041 mg, preferably about 1042 mg, preferably about 1043 mg, preferably about 1044 mg, preferably about 1045 mg, preferably about 1046 mg, preferably about 1047 mg, preferably about 1048 mg, preferably about 1049 mg, preferably about 1050 mg, preferably about 1051 mg, preferably about 1052 mg, preferably about 1053 mg, preferably about 1054 mg, preferably about 1055 mg, preferably about 1056 mg, preferably about 1057 mg, preferably about 1058 mg, preferably about 1059 mg, preferably about 1060 mg, preferably about 1061 mg, preferably about 1062 mg, preferably about 1063 mg, preferably about 1064 mg, preferably about 1065 mg, preferably about 1066 mg, preferably about 1067 mg, preferably about 1068 mg, preferably about 1069 mg, preferably about 1070 mg, preferably about 1071 mg, preferably about 1072 mg, preferably about 1073 mg, preferably about 1074 mg, preferably about 1075 mg, preferably about 1076 mg, preferably about 1077 mg, preferably about 1078 mg, preferably about 1079 mg, preferably about 1080 mg, preferably about 1081 mg, preferably about 1082 mg, preferably about 1083 mg, preferably about 1084 mg, preferably about 1085 mg, preferably about 1086 mg, preferably about 1087 mg, preferably about 1088 mg, preferably about 1089 mg, preferably about 1090 mg, preferably about 1091 mg, preferably about 1092 mg, preferably about 1093 mg, preferably about 1094 mg, preferably about 1095 mg, preferably about 1096 mg, preferably about 1097 mg, preferably about 1098 mg, preferably about 1099 mg, preferably about 1100 mg, preferably about 1101 mg, preferably about 1102 mg, preferably about 1103 mg, preferably about 1104 mg, preferably about 1105 mg, preferably about 1106 mg, preferably about 1107 mg, preferably about 1108 mg, preferably about 1109 mg, preferably about 1110 mg, preferably about 1111 mg, preferably about 1112 mg, preferably about 1113 mg, preferably about 1114 mg, preferably about 1115 mg, preferably about 1116 mg, preferably about 1117 mg, preferably about 1118 mg, preferably about 1119 mg, preferably about 1120 mg, preferably about 1121 mg, preferably about 1122 mg, preferably about 1123 mg, preferably about 1124 mg, preferably about 1125 mg, preferably about 1126 mg, preferably about 1127 mg, preferably about 1128 mg, preferably about 1129 mg, preferably about 1130 mg, preferably about 1131 mg, preferably about 1132 mg, preferably about 1133 mg, preferably about 1134 mg, preferably about 1135 mg, preferably about 1136 mg, preferably about 1137 mg, preferably about 1138 mg, preferably about 1139 mg, preferably about 1140 mg, preferably about 1141 mg, preferably about 1142 mg, preferably about 1143 mg, preferably about 1144 mg, preferably about 1145 mg, preferably about 1146 mg, preferably about 1147 mg, preferably about 1148 mg, preferably about 1149 mg, preferably about 1150 mg, preferably about 1151 mg, preferably about 1152 mg, preferably about 1153 mg, preferably about 1154 mg, preferably about 1155 mg, preferably about 1156 mg, preferably about 1157 mg, preferably about 1158 mg, preferably about 1159 mg, preferably about 1160 mg, preferably about 1161 mg, preferably about 1162 mg, preferably about 1163 mg, preferably about 1164 mg, preferably about 1165 mg, preferably about 1166 mg, preferably about 1167 mg, preferably about 1168 mg, preferably about 1169 mg, preferably about 1170 mg, preferably about 1171 mg, preferably about 1172 mg, preferably about 1173 mg, preferably about 1174 mg, preferably about 1175 mg, preferably about 1176 mg, preferably about 1177 mg, preferably about 1178 mg, preferably about 1179 mg, preferably about 1180 mg, preferably about 1181 mg, preferably about 1182 mg, preferably about 1183 mg, preferably about 1184 mg, preferably about 1185 mg, preferably about 1186 mg, preferably about 1187 mg, preferably about 1188 mg, preferably about 1189 mg, preferably about 1190 mg, preferably about 1191 mg, preferably about 1192 mg, preferably about 1193 mg, preferably about 1194 mg, preferably about 1195 mg, preferably about 1196 mg, preferably about 1197 mg, preferably about 1198 mg, preferably about 1199 mg, preferably about 1200 mg, preferably about 1201 mg, preferably about 1202 mg, preferably about 1203 mg, preferably about 1204 mg, preferably about 1205 mg, preferably about 1206 mg, preferably about 1207 mg, preferably about 1208 mg, preferably about 1209 mg, preferably about 1210 mg, preferably about 1211 mg, preferably about 1212 mg, preferably about 1213 mg, preferably about 1214 mg, preferably about 1215 mg, preferably about 1216 mg, preferably about 1217 mg, preferably about 1218 mg, preferably about 1219 mg, preferably about 1220 mg, preferably about 1221 mg, preferably about 1222 mg, preferably about 1223 mg, preferably about 1224 mg, preferably about 1225 mg, preferably about 1226 mg, preferably about 1227 mg, preferably about 1228 mg, preferably about 1229 mg, preferably about 1230 mg, preferably about 1231 mg, preferably about 1232 mg, preferably about 1233 mg, preferably about 1234 mg, preferably about 1235 mg, preferably about 1236 mg, preferably about 1237 mg, preferably about 1238 mg, preferably about 1239 mg, preferably about 1240 mg, preferably about 1241 mg, preferably about 1242 mg, preferably about 1243 mg, preferably about 1244 mg, preferably about 1245 mg, preferably about 1246 mg, preferably about 1247 mg, preferably about 1248 mg, preferably about 1249 mg, preferably about 1250 mg, preferably about 1251 mg, preferably about 1252 mg, preferably about 1253 mg, preferably about 1254 mg, preferably about 1255 mg, preferably about 1256 mg, preferably about 1257 mg, preferably about 1258 mg, preferably about 1259 mg, preferably about 1260 mg, preferably about 1261 mg, preferably about 1262 mg, preferably about 1263 mg, preferably about 1264 mg, preferably about 1265 mg, preferably about 1266 mg, preferably about 1267 mg, preferably about 1268 mg, preferably about 1269 mg, preferably about 1270 mg, preferably about 1271 mg, preferably about 1272 mg, preferably about 1273 mg, preferably about 1274 mg, preferably about 1275 mg, preferably about 1276 mg, preferably about 1277 mg, preferably about 1278 mg, preferably about 1279 mg, preferably about 1280 mg, preferably about 1281 mg, preferably about 1282 mg, preferably about 1283 mg, preferably about 1284 mg, preferably about 1285 mg, preferably about 1286 mg, preferably about 1287 mg, preferably about 1288 mg, preferably about 1289 mg, preferably about 1290 mg, preferably about 1291 mg, preferably about 1292 mg, preferably about 1293 mg, preferably about 1294 mg, preferably about 1295 mg, preferably about 1296 mg, preferably about 1297 mg, preferably about 1298 mg, preferably about 1299 mg, preferably about 1300 mg, preferably about 1301 mg, preferably about 1302 mg, preferably about 1303 mg, preferably about 1304 mg, preferably about 1305 mg, preferably about 1306 mg, preferably about 1307 mg, preferably about 1308 mg, preferably about 1309 mg, preferably about 1310 mg, preferably about 1311 mg, preferably about 1312 mg, preferably about 1313 mg, preferably about 1314 mg, preferably about 1315 mg, preferably about 1316 mg, preferably about 1317 mg, preferably about 1318 mg, preferably about 1319 mg, preferably about 1320 mg, preferably about 1321 mg, preferably about 1322 mg, preferably about 1323 mg, preferably about 1324 mg, preferably about 1325 mg, preferably about 1326 mg, preferably about 1327 mg, preferably about 1328 mg, preferably about 1329 mg, preferably about 1330 mg, preferably about 1331 mg, preferably about 1332 mg, preferably about 1333 mg, preferably about 1334 mg, preferably about 1335 mg, preferably about 1336 mg, preferably about 1337 mg, preferably about 1338 mg, preferably about 1339 mg, preferably about 1340 mg, preferably about 1341 mg, preferably about 1342 mg, preferably about 1343 mg, preferably about 1344 mg, preferably about 1345 mg, preferably about 1346 mg, preferably about 1347 mg, preferably about 1348 mg, preferably about 1349 mg, preferably about 1350 mg, preferably about 1351 mg, preferably about 1352 mg, preferably about 1353 mg, preferably about 1354 mg, preferably about 1355 mg, preferably about 1356 mg, preferably about 1357 mg, preferably about 1358 mg, preferably about 1359 mg, preferably about 1360 mg, preferably about 1361 mg, preferably about 1362 mg, preferably about 1363 mg, preferably about 1364 mg, preferably about 1365 mg, preferably about 1366 mg, preferably about 1367 mg, preferably about 1368 mg, preferably about 1369 mg, preferably about 1370 mg, preferably about 1371 mg, preferably about 1372 mg, preferably about 1373 mg, preferably about 1374 mg, preferably about 1375 mg, preferably about 1376 mg, preferably about 1377 mg, preferably about 1378 mg, preferably about 1379 mg, preferably about 1380 mg, preferably about 1381 mg, preferably about 1382 mg, preferably about 1383 mg, preferably about 1384 mg, preferably about 1385 mg, preferably about 1386 mg, preferably about 1387 mg, preferably about 1388 mg, preferably about 1389 mg, preferably about 1390 mg, preferably about 1391 mg, preferably about 1392 mg, preferably about 1393 mg, preferably about 1394 mg, preferably about 1395 mg, preferably about 1396 mg, preferably about 1397 mg, preferably about 1398 mg, preferably about 1399 mg, preferably about 1400 mg, preferably about 1401 mg, preferably about 1402 mg, preferably about 1403 mg, preferably about 1404 mg, preferably about 1405 mg, preferably about 1406 mg, preferably about 1407 mg, preferably about 1408 mg, preferably about 1409 mg, preferably about 1410 mg, preferably about 1411 mg, preferably about 1412 mg, preferably about 1413 mg, preferably about 1414 mg, preferably about 1415 mg, preferably about 1416 mg, preferably about 1417 mg, preferably about 1418 mg, preferably about 1419 mg, preferably about 1420 mg, preferably about 1421 mg, preferably about 1422 mg, preferably about 1423 mg, preferably about 1424 mg, preferably about 1425 mg, preferably about 1426 mg, preferably about 1427 mg, preferably about 1428 mg, preferably about 1429 mg, preferably about 1430 mg, preferably about 1431 mg, preferably about 1432 mg, preferably about 1433 mg, preferably about 1434 mg, preferably about 1435 mg, preferably about 1436 mg, preferably about 1437 mg, preferably about 1438 mg, preferably about 1439 mg, preferably about 1440 mg, preferably about 1441 mg, preferably about 1442 mg, preferably about 1443 mg, preferably about 1444 mg, preferably about 1445 mg, preferably about 1446 mg, preferably about 1447 mg, preferably about 1448 mg, preferably about 1449 mg, preferably about 1450 mg, preferably about 1451 mg, preferably about 1452 mg, preferably about 1453 mg, preferably about 1454 mg, preferably about 1455 mg, preferably about 1456 mg, preferably about 1457 mg, preferably about 1458 mg, preferably about 1459 mg, preferably about 1460 mg, preferably about 1461 mg, preferably about 1462 mg, preferably about 1463 mg, preferably about 1464 mg, preferably about 1465 mg, preferably about 1466 mg, preferably about 1467 mg, preferably about 1468 mg, preferably about 1469 mg, preferably about 1470 mg, preferably about 1471 mg, preferably about 1472 mg, preferably about 1473 mg, preferably about 1474 mg, preferably about 1475 mg, preferably about 1476 mg, preferably about 1477 mg, preferably about 1478 mg, preferably about 1479 mg, preferably about 1480 mg, preferably about 1481 mg, preferably about 1482 mg, preferably about 1483 mg, preferably about 1484 mg, preferably about 1485 mg, preferably about 1486 mg, preferably about 1487 mg, preferably about 1488 mg, preferably about 1489 mg, preferably about 1490 mg, preferably about 1491 mg, preferably about 1492 mg, preferably about 1493 mg, preferably about 1494 mg, preferably about 1495 mg, preferably about 1496 mg, preferably about 1497 mg, preferably about 1498 mg, preferably about 1499 mg, or more preferably about 1500 mg CBD or a pharmaceutically acceptable salt or derivative thereof per day.

In an embodiment, the subject is administered from about 20 mg to about 50 mg CBD or a pharmaceutically acceptable salt or derivative thereof per day.

In an embodiment the subject is administered from about 50 mg to about 500 mg CBD or a pharmaceutically acceptable salt or derivative thereof per day.

In an embodiment, the subject is administered from about 500 mg to about 1500 mg CBD or a pharmaceutically acceptable salt or derivative thereof per day.

In an embodiment, the subject is administered the CBD or a pharmaceutically acceptable salt or derivative thereof and/or the acetylcholinesterase inhibitor as escalating daily doses comprising a first daily dose and a second daily dose, wherein the amount of the CBD or a pharmaceutically acceptable salt or derivative thereof and/or the acetylcholinesterase inhibitor administered as the second daily dose is greater than the effective amount of the CBD or a pharmaceutically acceptable salt or derivative thereof and/or the acetylcholinesterase inhibitor administered as the first daily dose.

The amount and frequency of administration of escalating daily doses may be determined in accordance with methods of “empiric therapy” or “empirical administration.” The person skilled in the art would appreciate that empirical administration of escalating daily doses (e.g., first daily dose, second daily dose, third daily dose, fourth daily dose, fifth daily dose, and so on) will be determined by an appropriate medically trained professional overseeing the methods of treatment or prevention of dementia, as described herein.

In an embodiment, the first daily dose comprises from about 1 mg to about 1500 mg of the CBD or a pharmaceutically acceptable salt or derivative thereof and from about 1 mg to about 5 mg of the acetylcholinesterase inhibitor.

In an embodiment, the second daily dose comprises from about 1 mg to about 1500 mg of the CBD or a pharmaceutically acceptable salt or derivative thereof and from about 5 mg to about 10 mg of the acetylcholinesterase inhibitor.

In an embodiment, the third daily dose comprises from about 1 mg to about 1500 mg of the CBD or a pharmaceutically acceptable salt or derivative thereof and from about 10 mg to about 15 mg of the acetylcholinesterase inhibitor.

In an embodiment, the first daily dose comprises from about 20 mg to about 50 mg of the CBD or a pharmaceutically acceptable salt or derivative thereof and from about 1 mg to about 50 mg of the acetylcholinesterase inhibitor.

In an embodiment, the second daily dose comprises from about 50 mg to about 500 mg of the CBD or a pharmaceutically acceptable salt or derivative thereof and from about 1 mg to about 50 mg of the acetylcholinesterase inhibitor.

In an embodiment, the third daily dose comprises from about 500 mg to about 1500 mg of the CBD or a pharmaceutically acceptable salt or derivative thereof and from about 1 mg to about 50 mg of the acetylcholinesterase inhibitor.

The CBD or a pharmaceutically acceptable salt or derivative thereof and the acetylcholinesterase inhibitor disclosed herein may be administered to the subject by any suitable route that allows for delivery of the CBD or a pharmaceutically acceptable salt or derivative thereof and the acetylcholinesterase inhibitor to the subject at a therapeutically effective amount, as described herein. Suitable routes of administration will be known to persons skilled in the art, illustrative examples of which include enteral routes of administration (e.g., oral and rectal), parenteral routes of administration, typically by injection or microinjection (e.g., intramuscular, subcutaneous, intravenous, epidural, intra-articular, intraperitoneal, intracisternal or intrathecal) and topical (transdermal or transmucosal) routes of administration (e.g., buccal, sublingual, vaginal, intranasal or by inhalation).

In an embodiment, the CBD or a pharmaceutically acceptable salt or derivative thereof and the acetylcholinesterase inhibitor are orally administered to the subject.

Suitable dosage forms for oral administration would be known to persons skilled in the art, illustrative examples of which include tablets, aqueous or oily suspensions, lozenges, troches, powders, granules, emulsions, capsules, liquids, syrups or elixirs.

In an embodiment, the CBD or a pharmaceutically acceptable salt or derivative thereof is in liquid, oil, tablet or capsule form. In another embodiment, the CBD or a pharmaceutically acceptable salt or derivative thereof is in liquid form.

In an embodiment, the acetylcholinesterase inhibitor is in liquid, oil, tablet or capsule form. In another embodiment, the acetylcholinesterase inhibitor is in tablet or capsule form.

It is further contemplated herein that the CBD or a pharmaceutically acceptable salt or derivative thereof, and the acetylcholinesterase inhibitor may be co-administered with one or more other appropriate therapeutic agents, illustrative examples of which include anti-inflammatory agents, antipsychotic agents, and analgesics.

In accordance with the methods disclosed herein, the CBD or a pharmaceutically acceptable salt or derivative thereof, and the acetylcholinesterase inhibitor may be co-administered as separate compositions.

In an embodiment, the CBD or a pharmaceutically acceptable salt or derivative thereof, and the acetylcholinesterase inhibitor are administered sequentially.

By “sequential” administration it is meant there is an interval between the administration of the CBD or a pharmaceutically acceptable salt or derivative thereof, and the acetylcholinesterase inhibitor. The interval between sequential administrations may be seconds, minutes, hours or days. In a preferred embodiment, the interval between sequential administrations of the CBD or a pharmaceutically acceptable salt or derivative thereof, and the acetylcholinesterase inhibitor is less than an hour, preferably less than 30 minutes, most preferably less than 1 minute. Sequential administration may be in any order (i.e., administration of CBD or a pharmaceutically acceptable salt or derivative thereof prior to the administration of the acetylcholinesterase inhibitor, or administration of the acetylcholinesterase inhibitor prior to the administration of the CBD or a pharmaceutically acceptable salt or derivative thereof).

In another embodiment, the CBD or a pharmaceutically acceptable salt or derivative thereof, and the acetylcholinesterase inhibitor are administered simultaneously.

By “simultaneous” administration it is meant that the CBD or a pharmaceutically acceptable salt or derivative thereof, and the acetylcholinesterase inhibitor are administered at the same time. In accordance with the methods disclosed herein, the CBD or a pharmaceutically acceptable salt or derivative thereof, and the acetylcholinesterase inhibitor may be administered simultaneously as two separate compositions or dosage forms. Alternatively, the CBD or a pharmaceutically acceptable salt or derivative thereof, and the acetylcholinesterase inhibitor are administered simultaneously as a single composition or dosage form.

In some embodiments, periodic re-administration of the active agents (either sequentially or simultaneously) may be required to achieve a desirable therapeutic effect. The exact amounts and rates of administration of the CBD or a pharmaceutically acceptable salt or derivative thereof, and the acetylcholinesterase inhibitor will depend on a number of factors, examples of which are described elsewhere herein, such as the subject's age, body weight, general health, sex and dietary requirements, as well as any drugs or agents used in combination or coincidental with the administration of the composition. Where multiple divided doses are required, these may be administered hourly, daily, weekly, monthly or at other suitable time intervals or the dose may be proportionally reduced as indicated by the exigencies of the situation.

In an embodiment, the CBD or a pharmaceutically acceptable salt or derivative thereof and the acetylcholinesterase inhibitor are provided to the subject in a single daily dose.

In an embodiment, the CBD or a pharmaceutically acceptable salt or derivative thereof and the acetylcholinesterase inhibitor are administered to the subject between about 10 minutes and about 120 minutes before sleep (e.g., 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, or 120 minutes before sleep).

Thus, in an embodiment the CBD or a pharmaceutically acceptable salt or derivative thereof and the acetylcholinesterase inhibitor are administered to the subject between about 10 minutes and about 120 minutes before sleep, preferably about 10 minutes, preferably about 11 minutes, preferably about 12 minutes, preferably about 13 minutes, preferably about 14 minutes, preferably about 15 minutes, preferably about 16 minutes, preferably about 17 minutes, preferably about 18 minutes, preferably about 19 minutes, preferably about 20 minutes, preferably about 21 minutes, preferably about 22 minutes, preferably about 23 minutes, preferably about 24 minutes, preferably about 25 minutes, preferably about 26 minutes, preferably about 27 minutes, preferably about 28 minutes, preferably about 29 minutes, preferably about 30 minutes, preferably about 31 minutes, preferably about 32 minutes, preferably about 33 minutes, preferably about 34 minutes, preferably about 35 minutes, preferably about 36 minutes, preferably about 37 minutes, preferably about 38 minutes, preferably about 39 minutes, preferably about 40 minutes, preferably about 41 minutes, preferably about 42 minutes, preferably about 43 minutes, preferably about 44 minutes, preferably about 45 minutes, preferably about 46 minutes, preferably about 47 minutes, preferably about 48 minutes, preferably about 49 minutes, preferably about 50 minutes, preferably about 51 minutes, preferably about 52 minutes, preferably about 53 minutes, preferably about 54 minutes, preferably about 55 minutes, preferably about 56 minutes, preferably about 57 minutes, preferably about 58 minutes, preferably about 59 minutes, preferably about 60 minutes, preferably about 61 minutes, preferably about 62 minutes, preferably about 63 minutes, preferably about 64 minutes, preferably about 65 minutes, preferably about 66 minutes, preferably about 67 minutes, preferably about 68 minutes, preferably about 69 minutes, preferably about 70 minutes, preferably about 71 minutes, preferably about 72 minutes, preferably about 73 minutes, preferably about 74 minutes, preferably about 75 minutes, preferably about 76 minutes, preferably about 77 minutes, preferably about 78 minutes, preferably about 79 minutes, preferably about 80 minutes, preferably about 81 minutes, preferably about 82 minutes, preferably about 83 minutes, preferably about 84 minutes, preferably about 85 minutes, preferably about 86 minutes, preferably about 87 minutes, preferably about 88 minutes, preferably about 89 minutes, preferably about 90 minutes, preferably about 91 minutes, preferably about 92 minutes, preferably about 93 minutes, preferably about 94 minutes, preferably about 95 minutes, preferably about 96 minutes, preferably about 97 minutes, preferably about 98 minutes, preferably about 99 minutes, preferably about 100 minutes, preferably about 101 minutes, preferably about 102 minutes, preferably about 103 minutes, preferably about 104 minutes, preferably about 105 minutes, preferably about 106 minutes, preferably about 107 minutes, preferably about 108 minutes, preferably about 109 minutes, preferably about 110 minutes, preferably about 111 minutes, preferably about 112 minutes, preferably about 113 minutes, preferably about 114 minutes, preferably about 115 minutes, preferably about 116 minutes, preferably about 117 minutes, preferably about 118 minutes, preferably about 119 minutes, or more preferably about 120 minutes before sleep.

In another embodiment, the CBD or a pharmaceutically acceptable salt or derivative thereof and the acetylcholinesterase inhibitor are administered to the subject between about 30 minutes and about 60 minutes before sleep.

In other embodiments disclosed herein, the CBD or a pharmaceutically acceptable salt or derivative thereof and the acetylcholinesterase inhibitor are formulated as separate unit dosage forms for administration. The unit dosage form may be suitable for oral administration, e.g., solution, capsule or tablet form. Those skilled in the art will appreciate that unit dosage forms comprising the CBD or a pharmaceutically acceptable salt or derivative thereof and the acetylcholinesterase inhibitor need not be of the same type. Thus, methods of the present disclosure contemplate the administration of, for example, CBD in oil form and the acetylcholinesterase inhibitor in capsule or tablet form.

In an embodiment, the method further comprises the administration of one or more additional therapeutic agents selected from the group consisting of anti-inflammatory agents, antipsychotic agents, and analgesics.

In an embodiment, the method further comprises the administration of one or more additional cannabinoids.

In an embodiment, the one or more additional cannabinoids are selected from the group consisting of CBG, CBN, CBDL, CBC and THC.

In an aspect disclosed herein, there is provided a use of CBD or a pharmaceutically acceptable salt or derivative thereof and an acetylcholinesterase inhibitor in the manufacture of a medicament for the treatment or prevention of at least one neurodegenerative disorder and/or dementia.

In an aspect disclosed herein, there is provided a use of CBD or a pharmaceutically acceptable salt or derivative thereof and an acetylcholinesterase inhibitor in the manufacture of a medicament for the treatment or prevention of at least one neurodegenerative disorder.

In an aspect disclosed herein, there is provided a use of CBD or a pharmaceutically acceptable salt or derivative thereof and an acetylcholinesterase inhibitor in the manufacture of a medicament for the treatment or prevention of dementia.

In an embodiment, the medicament comprises from about 1 mg to about 1500 mg CBD or a pharmaceutically acceptable salt or derivative thereof per dose.

In an embodiment, the acetylcholinesterase inhibitor is selected from the group consisting of donepezil, rivastigmine and galantamine

In an embodiment, the acetylcholinesterase inhibitor is donepezil, or a pharmaceutically acceptable salt thereof.

In an embodiment, the acetylcholinesterase inhibitor is donepezil hydrochloride.

In an aspect disclosed herein, there is provided CBD or a pharmaceutically acceptable salt or derivative thereof and an acetylcholinesterase inhibitor for use in a method for the treatment or prevention of at least one neurodegenerative disorder and/or dementia.

In an aspect disclosed herein, there is provided CBD or a pharmaceutically acceptable salt or derivative thereof and an acetylcholinesterase inhibitor for use in a method for the treatment or prevention of at least one neurodegenerative disorder.

In an aspect disclosed herein, there is provided CBD or a pharmaceutically acceptable salt or derivative thereof and an acetylcholinesterase inhibitor for use in a method for the treatment or prevention of dementia.

In an embodiment, the CBD or a pharmaceutically acceptable salt or derivative thereof is to be administered at a dose of from about 1 mg to about 1500 mg.

In an embodiment, the acetylcholinesterase inhibitor is selected from the group consisting of donepezil, rivastigmine and galantamine.

In an embodiment, the acetylcholinesterase inhibitor is donepezil, or a pharmaceutically acceptable salt thereof.

In an embodiment, the acetylcholinesterase inhibitor is donepezil hydrochloride.

In an embodiment, the dementia is selected from the group consisting of vascular dementia, dementia with Lewy bodies, frontotemporal dementia, HIV-induced dementia, and traumatic brain injury (TBI) with dementia.

In an embodiment, the at least one neurodegenerative disorder selected from the group consisting of Alzheimer's disease, Parkinson's disease, Huntington's disease, chronic traumatic encephalopathy (CTE), amyotrophic lateral sclerosis (ALS), neurodegeneration with brain iron accumulation, mitochondrial dysfunction, Huntington's disease, ataxia, and neuronal ceroid lipofuscinosis (NCL).

In an embodiment, the at least one neurodegenerative disorder is selected from the group consisting of Parkinson's disease, Alzheimer's disease, Huntington's disease and ALS.

In an embodiment, the at least one neurodegenerative disorder is Alzheimer's disease.

Methods for the Treatment or Prevention of Diseases Associated with Glutamate-Mediated Excitotoxicity

As disclosed elsewhere herein, the present disclosure provides a method for the treatment or prevention of a disease associated with glutamate-mediated excitotoxicity, the method comprising administering to a subject in need thereof: (i) cannabidiol (CBD) or a pharmaceutically acceptable salt or derivative thereof, and (ii) an acetylcholinesterase inhibitor, wherein the subject is administered from about 1 mg to about 1500 mg CBD or a pharmaceutically acceptable salt or derivative thereof per day.

In an embodiment, the subject is administered from about 1 mg to about 50 mg of the acetylcholinesterase inhibitor per day (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg of the acetylcholinesterase inhibitor per day).

In an embodiment, the subject is administered from about 5 mg to about 10 mg of the acetylcholinesterase inhibitor per day.

In an embodiment, the subject is administered from about 10 mg to about 25 mg of the acetylcholinesterase inhibitor per day.

In an embodiment, the acetylcholinesterase inhibitor is donepezil or a pharmaceutically acceptable salt thereof, and wherein the subject is administered from about 5 mg to about 10 mg of the donepezil or a pharmaceutically acceptable salt thereof per day.

In an embodiment, the acetylcholinesterase inhibitor is donepezil or a pharmaceutically acceptable salt thereof, and wherein the subject is administered from about 10 mg to about 25 mg of the donepezil or a pharmaceutically acceptable salt thereof per day.

In an embodiment, the acetylcholinesterase inhibitor is donepezil hydrochloride or a pharmaceutically acceptable salt thereof, and wherein the subject is administered from about 5 mg to about 10 mg of the donepezil or a pharmaceutically acceptable salt thereof per day.

In an embodiment, the acetylcholinesterase inhibitor is donepezil hydrochloride or a pharmaceutically acceptable salt thereof, and wherein the subject is administered from about 10 mg to about 25 mg of the donepezil or a pharmaceutically acceptable salt thereof per day.

In an embodiment, the acetylcholinesterase inhibitor is rivastigmine or a pharmaceutically acceptable salt thereof, and wherein the subject is administered from about 1 mg to about 20 mg of the rivastigmine or a pharmaceutically acceptable salt thereof per day.

In an embodiment, the acetylcholinesterase inhibitor is rivastigmine or a pharmaceutically acceptable salt thereof, and wherein the subject is administered from about 3 mg to about 12 mg of the rivastigmine or a pharmaceutically acceptable salt thereof per day.

In an embodiment, the acetylcholinesterase inhibitor is galantamine or a pharmaceutically acceptable salt thereof, and wherein the subject is administered from about 5 mg to about 50 mg of the galantamine or a pharmaceutically acceptable salt thereof per day.

In an embodiment, the acetylcholinesterase inhibitor is galantamine or a pharmaceutically acceptable salt thereof, and wherein the subject is administered from about 8 mg to about 24 mg of the galantamine or a pharmaceutically acceptable salt thereof per day.

In an embodiment, the subject is administered from about 1 mg to about 1500 mg CBD or a pharmaceutically acceptable salt or derivative thereof per day (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, 1000 mg, 1001 mg, 1002 mg, 1003 mg, 1004 mg, 1005 mg, 1006 mg, 1007 mg, 1008 mg, 1009 mg, 1010 mg, 1011 mg, 1012 mg, 1013 mg, 1014 mg, 1015 mg, 1016 mg, 1017 mg, 1018 mg, 1019 mg, 1020 mg, 1021 mg, 1022 mg, 1023 mg, 1024 mg, 1025 mg, 1026 mg, 1027 mg, 1028 mg, 1029 mg, 1030 mg, 1031 mg, 1032 mg, 1033 mg, 1034 mg, 1035 mg, 1036 mg, 1037 mg, 1038 mg, 1039 mg, 1040 mg, 1041 mg, 1042 mg, 1043 mg, 1044 mg, 1045 mg, 1046 mg, 1047 mg, 1048 mg, 1049 mg, 1050 mg, 1051 mg, 1052 mg, 1053 mg, 1054 mg, 1055 mg, 1056 mg, 1057 mg, 1058 mg, 1059 mg, 1060 mg, 1061 mg, 1062 mg, 1063 mg, 1064 mg, 1065 mg, 1066 mg, 1067 mg, 1068 mg, 1069 mg, 1070 mg, 1071 mg, 1072 mg, 1073 mg, 1074 mg, 1075 mg, 1076 mg, 1077 mg, 1078 mg, 1079 mg, 1080 mg, 1081 mg, 1082 mg, 1083 mg, 1084 mg, 1085 mg, 1086 mg, 1087 mg, 1088 mg, 1089 mg, 1090 mg, 1091 mg, 1092 mg, 1093 mg, 1094 mg, 1095 mg, 1096 mg, 1097 mg, 1098 mg, 1099 mg, 1100 mg, 1101 mg, 1102 mg, 1103 mg, 1104 mg, 1105 mg, 1106 mg, 1107 mg, 1108 mg, 1109 mg, 1110 mg, 1111 mg, 1112 mg, 1113 mg, 1114 mg, 1115 mg, 1116 mg, 1117 mg, 1118 mg, 1119 mg, 1120 mg, 1121 mg, 1122 mg, 1123 mg, 1124 mg, 1125 mg, 1126 mg, 1127 mg, 1128 mg, 1129 mg, 1130 mg, 1131 mg, 1132 mg, 1133 mg, 1134 mg, 1135 mg, 1136 mg, 1137 mg, 1138 mg, 1139 mg, 1140 mg, 1141 mg, 1142 mg, 1143 mg, 1144 mg, 1145 mg, 1146 mg, 1147 mg, 1148 mg, 1149 mg, 1150 mg, 1151 mg, 1152 mg, 1153 mg, 1154 mg, 1155 mg, 1156 mg, 1157 mg, 1158 mg, 1159 mg, 1160 mg, 1161 mg, 1162 mg, 1163 mg, 1164 mg, 1165 mg, 1166 mg, 1167 mg, 1168 mg, 1169 mg, 1170 mg, 1171 mg, 1172 mg, 1173 mg, 1174 mg, 1175 mg, 1176 mg, 1177 mg, 1178 mg, 1179 mg, 1180 mg, 1181 mg, 1182 mg, 1183 mg, 1184 mg, 1185 mg, 1186 mg, 1187 mg, 1188 mg, 1189 mg, 1190 mg, 1191 mg, 1192 mg, 1193 mg, 1194 mg, 1195 mg, 1196 mg, 1197 mg, 1198 mg, 1199 mg, 1200 mg, 1201 mg, 1202 mg, 1203 mg, 1204 mg, 1205 mg, 1206 mg, 1207 mg, 1208 mg, 1209 mg, 1210 mg, 1211 mg, 1212 mg, 1213 mg, 1214 mg, 1215 mg, 1216 mg, 1217 mg, 1218 mg, 1219 mg, 1220 mg, 1221 mg, 1222 mg, 1223 mg, 1224 mg, 1225 mg, 1226 mg, 1227 mg, 1228 mg, 1229 mg, 1230 mg, 1231 mg, 1232 mg, 1233 mg, 1234 mg, 1235 mg, 1236 mg, 1237 mg, 1238 mg, 1239 mg, 1240 mg, 1241 mg, 1242 mg, 1243 mg, 1244 mg, 1245 mg, 1246 mg, 1247 mg, 1248 mg, 1249 mg, 1250 mg, 1251 mg, 1252 mg, 1253 mg, 1254 mg, 1255 mg, 1256 mg, 1257 mg, 1258 mg, 1259 mg, 1260 mg, 1261 mg, 1262 mg, 1263 mg, 1264 mg, 1265 mg, 1266 mg, 1267 mg, 1268 mg, 1269 mg, 1270 mg, 1271 mg, 1272 mg, 1273 mg, 1274 mg, 1275 mg, 1276 mg, 1277 mg, 1278 mg, 1279 mg, 1280 mg, 1281 mg, 1282 mg, 1283 mg, 1284 mg, 1285 mg, 1286 mg, 1287 mg, 1288 mg, 1289 mg, 1290 mg, 1291 mg, 1292 mg, 1293 mg, 1294 mg, 1295 mg, 1296 mg, 1297 mg, 1298 mg, 1299 mg, 1300 mg, 1301 mg, 1302 mg, 1303 mg, 1304 mg, 1305 mg, 1306 mg, 1307 mg, 1308 mg, 1309 mg, 1310 mg, 1311 mg, 1312 mg, 1313 mg, 1314 mg, 1315 mg, 1316 mg, 1317 mg, 1318 mg, 1319 mg, 1320 mg, 1321 mg, 1322 mg, 1323 mg, 1324 mg, 1325 mg, 1326 mg, 1327 mg, 1328 mg, 1329 mg, 1330 mg, 1331 mg, 1332 mg, 1333 mg, 1334 mg, 1335 mg, 1336 mg, 1337 mg, 1338 mg, 1339 mg, 1340 mg, 1341 mg, 1342 mg, 1343 mg, 1344 mg, 1345 mg, 1346 mg, 1347 mg, 1348 mg, 1349 mg, 1350 mg, 1351 mg, 1352 mg, 1353 mg, 1354 mg, 1355 mg, 1356 mg, 1357 mg, 1358 mg, 1359 mg, 1360 mg, 1361 mg, 1362 mg, 1363 mg, 1364 mg, 1365 mg, 1366 mg, 1367 mg, 1368 mg, 1369 mg, 1370 mg, 1371 mg, 1372 mg, 1373 mg, 1374 mg, 1375 mg, 1376 mg, 1377 mg, 1378 mg, 1379 mg, 1380 mg, 1381 mg, 1382 mg, 1383 mg, 1384 mg, 1385 mg, 1386 mg, 1387 mg, 1388 mg, 1389 mg, 1390 mg, 1391 mg, 1392 mg, 1393 mg, 1394 mg, 1395 mg, 1396 mg, 1397 mg, 1398 mg, 1399 mg, 1400 mg, 1401 mg, 1402 mg, 1403 mg, 1404 mg, 1405 mg, 1406 mg, 1407 mg, 1408 mg, 1409 mg, 1410 mg, 1411 mg, 1412 mg, 1413 mg, 1414 mg, 1415 mg, 1416 mg, 1417 mg, 1418 mg, 1419 mg, 1420 mg, 1421 mg, 1422 mg, 1423 mg, 1424 mg, 1425 mg, 1426 mg, 1427 mg, 1428 mg, 1429 mg, 1430 mg, 1431 mg, 1432 mg, 1433 mg, 1434 mg, 1435 mg, 1436 mg, 1437 mg, 1438 mg, 1439 mg, 1440 mg, 1441 mg, 1442 mg, 1443 mg, 1444 mg, 1445 mg, 1446 mg, 1447 mg, 1448 mg, 1449 mg, 1450 mg, 1451 mg, 1452 mg, 1453 mg, 1454 mg, 1455 mg, 1456 mg, 1457 mg, 1458 mg, 1459 mg, 1460 mg, 1461 mg, 1462 mg, 1463 mg, 1464 mg, 1465 mg, 1466 mg, 1467 mg, 1468 mg, 1469 mg, 1470 mg, 1471 mg, 1472 mg, 1473 mg, 1474 mg, 1475 mg, 1476 mg, 1477 mg, 1478 mg, 1479 mg, 1480 mg, 1481 mg, 1482 mg, 1483 mg, 1484 mg, 1485 mg, 1486 mg, 1487 mg, 1488 mg, 1489 mg, 1490 mg, 1491 mg, 1492 mg, 1493 mg, 1494 mg, 1495 mg, 1496 mg, 1497 mg, 1498 mg, 1499 mg, or 1500 mg CBD or a pharmaceutically acceptable salt or derivative thereof per day).

In an embodiment, the subject is administered from about 20 mg to about 50 mg CBD or a pharmaceutically acceptable salt or derivative thereof per day.

In an embodiment the subject is administered from about 50 mg to about 500 mg CBD or a pharmaceutically acceptable salt or derivative thereof per day.

In an embodiment, the subject is administered from about 500 mg to about 1500 mg CBD or a pharmaceutically acceptable salt or derivative thereof per day.

In an embodiment, the CBD or a pharmaceutically acceptable salt or derivative thereof and the acetylcholinesterase inhibitor are orally administered to the subject.

In an embodiment, the CBD or a pharmaceutically acceptable salt or derivative thereof is in liquid, oil, tablet or capsule form. In another embodiment, the CBD or a pharmaceutically acceptable salt or derivative thereof is in liquid form.

In an embodiment, the acetylcholinesterase inhibitor is in liquid, oil, tablet or capsule form. In another embodiment, the acetylcholinesterase inhibitor is in tablet or capsule form.

In an embodiment, the CBD or a pharmaceutically acceptable salt or derivative thereof, and the acetylcholinesterase inhibitor may be co-administered with one or more other appropriate therapeutic agents, illustrative examples of which include anti-inflammatory agents, antipsychotic agents, and analgesics.

In an embodiment, the CBD or a pharmaceutically acceptable salt or derivative thereof, and the acetylcholinesterase inhibitor may be co-administered as separate compositions.

In an embodiment, the CBD or a pharmaceutically acceptable salt or derivative thereof, and the acetylcholinesterase inhibitor are administered sequentially.

In another embodiment, the CBD or a pharmaceutically acceptable salt or derivative thereof, and the acetylcholinesterase inhibitor are administered simultaneously.

In an embodiment, the CBD or a pharmaceutically acceptable salt or derivative thereof and the acetylcholinesterase inhibitor are provided to the subject in a single daily dose.

In an embodiment, the method further comprises the administration of one or more additional therapeutic agents selected from the group consisting of anti-inflammatory agents, antipsychotic agents, and analgesics.

In an embodiment, the method further comprises the administration of one or more additional cannabinoids.

In an embodiment, the one or more additional cannabinoids are selected from the group consisting of CBG, CBN, CBDL, CBC and THC.

In an embodiment, the disease associated with glutamate-mediated excitotoxicity is an acute disease selected from the group consisting of ischemia, TBI, epilepsy, hypoglycaemia, and status epilepticus.

In an embodiment, the disease associated with glutamate-mediated excitotoxicity is a chronic neurodegenerative disorder selected from the group consisting of Parkinson's disease, Alzheimer's disease, Huntington's disease and amyotrophic lateral sclerosis (ALS).

In an aspect disclosed herein, there is provided a use of CBD or a pharmaceutically acceptable salt or derivative thereof and an acetylcholinesterase inhibitor in the manufacture of a medicament for the treatment or prevention of a disease associated with glutamate-mediated excitotoxicity.

In an embodiment, the medicament comprises from about 1 mg to about 1500 mg CBD or a pharmaceutically acceptable salt or derivative thereof per dose.

In an embodiment, the acetylcholinesterase inhibitor is selected from the group consisting of donepezil, rivastigmine and galantamine.

In an embodiment, the acetylcholinesterase inhibitor is donepezil, or a pharmaceutically acceptable salt thereof.

In an embodiment, the acetylcholinesterase inhibitor is donepezil hydrochloride.

In an embodiment, the disease associated with glutamate-mediated excitotoxicity is an acute disease selected from the group consisting of ischemia, TBI, epilepsy, hypoglycaemia, and status epilepticus.

In an embodiment, the disease associated with glutamate-mediated excitotoxicity is a chronic neurodegenerative disorder selected from the group consisting of Parkinson's disease, Alzheimer's disease, Huntington's disease and amyotrophic lateral sclerosis (ALS).

In an aspect disclosed herein, there is provided CBD or a pharmaceutically acceptable salt or derivative thereof and an acetylcholinesterase inhibitor for use in a method for the treatment or prevention of a disease associated with glutamate-mediated excitotoxicity.

In an embodiment, the CBD or a pharmaceutically acceptable salt or derivative thereof is to be administered at a dose of from about 1 mg to about 1500 mg.

In an embodiment, the acetylcholinesterase inhibitor is selected from the group consisting of donepezil, rivastigmine and galantamine.

In an embodiment, the acetylcholinesterase inhibitor is donepezil, or a pharmaceutically acceptable salt thereof.

In an embodiment, the acetylcholinesterase inhibitor is donepezil hydrochloride.

In an embodiment, the disease associated with glutamate-mediated excitotoxicity is an acute disease selected from the group consisting of ischemia, TBI, epilepsy, hypoglycaemia, and status epilepticus.

In an embodiment, the disease associated with glutamate-mediated excitotoxicity is a chronic neurodegenerative disorder selected from the group consisting of Parkinson's disease, Alzheimer's disease, Huntington's disease and amyotrophic lateral sclerosis (ALS).

### Pharmaceutical Compositions

In an aspect disclosed herein, there is provided a pharmaceutical composition comprising CBD or a pharmaceutically acceptable salt thereof, and an acetylcholinesterase inhibitor.

In an embodiment, the composition comprises from about 1 mg to about 1500 mg CBD or a pharmaceutically acceptable salt or derivative thereof per dose (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, 1000 mg, 1001 mg, 1002 mg, 1003 mg, 1004 mg, 1005 mg, 1006 mg, 1007 mg, 1008 mg, 1009 mg, 1010 mg, 1011 mg, 1012 mg, 1013 mg, 1014 mg, 1015 mg, 1016 mg, 1017 mg, 1018 mg, 1019 mg, 1020 mg, 1021 mg, 1022 mg, 1023 mg, 1024 mg, 1025 mg, 1026 mg, 1027 mg, 1028 mg, 1029 mg, 1030 mg, 1031 mg, 1032 mg, 1033 mg, 1034 mg, 1035 mg, 1036 mg, 1037 mg, 1038 mg, 1039 mg, 1040 mg, 1041 mg, 1042 mg, 1043 mg, 1044 mg, 1045 mg, 1046 mg, 1047 mg, 1048 mg, 1049 mg, 1050 mg, 1051 mg, 1052 mg, 1053 mg, 1054 mg, 1055 mg, 1056 mg, 1057 mg, 1058 mg, 1059 mg, 1060 mg, 1061 mg, 1062 mg, 1063 mg, 1064 mg, 1065 mg, 1066 mg, 1067 mg, 1068 mg, 1069 mg, 1070 mg, 1071 mg, 1072 mg, 1073 mg, 1074 mg, 1075 mg, 1076 mg, 1077 mg, 1078 mg, 1079 mg, 1080 mg, 1081 mg, 1082 mg, 1083 mg, 1084 mg, 1085 mg, 1086 mg, 1087 mg, 1088 mg, 1089 mg, 1090 mg, 1091 mg, 1092 mg, 1093 mg, 1094 mg, 1095 mg, 1096 mg, 1097 mg, 1098 mg, 1099 mg, 1100 mg, 1101 mg, 1102 mg, 1103 mg, 1104 mg, 1105 mg, 1106 mg, 1107 mg, 1108 mg, 1109 mg, 1110 mg, 1111 mg, 1112 mg, 1113 mg, 1114 mg, 1115 mg, 1116 mg, 1117 mg, 1118 mg, 1119 mg, 1120 mg, 1121 mg, 1122 mg, 1123 mg, 1124 mg, 1125 mg, 1126 mg, 1127 mg, 1128 mg, 1129 mg, 1130 mg, 1131 mg, 1132 mg, 1133 mg, 1134 mg, 1135 mg, 1136 mg, 1137 mg, 1138 mg, 1139 mg, 1140 mg, 1141 mg, 1142 mg, 1143 mg, 1144 mg, 1145 mg, 1146 mg, 1147 mg, 1148 mg, 1149 mg, 1150 mg, 1151 mg, 1152 mg, 1153 mg, 1154 mg, 1155 mg, 1156 mg, 1157 mg, 1158 mg, 1159 mg, 1160 mg, 1161 mg, 1162 mg, 1163 mg, 1164 mg, 1165 mg, 1166 mg, 1167 mg, 1168 mg, 1169 mg, 1170 mg, 1171 mg, 1172 mg, 1173 mg, 1174 mg, 1175 mg, 1176 mg, 1177 mg, 1178 mg, 1179 mg, 1180 mg, 1181 mg, 1182 mg, 1183 mg, 1184 mg, 1185 mg, 1186 mg, 1187 mg, 1188 mg, 1189 mg, 1190 mg, 1191 mg, 1192 mg, 1193 mg, 1194 mg, 1195 mg, 1196 mg, 1197 mg, 1198 mg, 1199 mg, 1200 mg, 1201 mg, 1202 mg, 1203 mg, 1204 mg, 1205 mg, 1206 mg, 1207 mg, 1208 mg, 1209 mg, 1210 mg, 1211 mg, 1212 mg, 1213 mg, 1214 mg, 1215 mg, 1216 mg, 1217 mg, 1218 mg, 1219 mg, 1220 mg, 1221 mg, 1222 mg, 1223 mg, 1224 mg, 1225 mg, 1226 mg, 1227 mg, 1228 mg, 1229 mg, 1230 mg, 1231 mg, 1232 mg, 1233 mg, 1234 mg, 1235 mg, 1236 mg, 1237 mg, 1238 mg, 1239 mg, 1240 mg, 1241 mg, 1242 mg, 1243 mg, 1244 mg, 1245 mg, 1246 mg, 1247 mg, 1248 mg, 1249 mg, 1250 mg, 1251 mg, 1252 mg, 1253 mg, 1254 mg, 1255 mg, 1256 mg, 1257 mg, 1258 mg, 1259 mg, 1260 mg, 1261 mg, 1262 mg, 1263 mg, 1264 mg, 1265 mg, 1266 mg, 1267 mg, 1268 mg, 1269 mg, 1270 mg, 1271 mg, 1272 mg, 1273 mg, 1274 mg, 1275 mg, 1276 mg, 1277 mg, 1278 mg, 1279 mg, 1280 mg, 1281 mg, 1282 mg, 1283 mg, 1284 mg, 1285 mg, 1286 mg, 1287 mg, 1288 mg, 1289 mg, 1290 mg, 1291 mg, 1292 mg, 1293 mg, 1294 mg, 1295 mg, 1296 mg, 1297 mg, 1298 mg, 1299 mg, 1300 mg, 1301 mg, 1302 mg, 1303 mg, 1304 mg, 1305 mg, 1306 mg, 1307 mg, 1308 mg, 1309 mg, 1310 mg, 1311 mg, 1312 mg, 1313 mg, 1314 mg, 1315 mg, 1316 mg, 1317 mg, 1318 mg, 1319 mg, 1320 mg, 1321 mg, 1322 mg, 1323 mg, 1324 mg, 1325 mg, 1326 mg, 1327 mg, 1328 mg, 1329 mg, 1330 mg, 1331 mg, 1332 mg, 1333 mg, 1334 mg, 1335 mg, 1336 mg, 1337 mg, 1338 mg, 1339 mg, 1340 mg, 1341 mg, 1342 mg, 1343 mg, 1344 mg, 1345 mg, 1346 mg, 1347 mg, 1348 mg, 1349 mg, 1350 mg, 1351 mg, 1352 mg, 1353 mg, 1354 mg, 1355 mg, 1356 mg, 1357 mg, 1358 mg, 1359 mg, 1360 mg, 1361 mg, 1362 mg, 1363 mg, 1364 mg, 1365 mg, 1366 mg, 1367 mg, 1368 mg, 1369 mg, 1370 mg, 1371 mg, 1372 mg, 1373 mg, 1374 mg, 1375 mg, 1376 mg, 1377 mg, 1378 mg, 1379 mg, 1380 mg, 1381 mg, 1382 mg, 1383 mg, 1384 mg, 1385 mg, 1386 mg, 1387 mg, 1388 mg, 1389 mg, 1390 mg, 1391 mg, 1392 mg, 1393 mg, 1394 mg, 1395 mg, 1396 mg, 1397 mg, 1398 mg, 1399 mg, 1400 mg, 1401 mg, 1402 mg, 1403 mg, 1404 mg, 1405 mg, 1406 mg, 1407 mg, 1408 mg, 1409 mg, 1410 mg, 1411 mg, 1412 mg, 1413 mg, 1414 mg, 1415 mg, 1416 mg, 1417 mg, 1418 mg, 1419 mg, 1420 mg, 1421 mg, 1422 mg, 1423 mg, 1424 mg, 1425 mg, 1426 mg, 1427 mg, 1428 mg, 1429 mg, 1430 mg, 1431 mg, 1432 mg, 1433 mg, 1434 mg, 1435 mg, 1436 mg, 1437 mg, 1438 mg, 1439 mg, 1440 mg, 1441 mg, 1442 mg, 1443 mg, 1444 mg, 1445 mg, 1446 mg, 1447 mg, 1448 mg, 1449 mg, 1450 mg, 1451 mg, 1452 mg, 1453 mg, 1454 mg, 1455 mg, 1456 mg, 1457 mg, 1458 mg, 1459 mg, 1460 mg, 1461 mg, 1462 mg, 1463 mg, 1464 mg, 1465 mg, 1466 mg, 1467 mg, 1468 mg, 1469 mg, 1470 mg, 1471 mg, 1472 mg, 1473 mg, 1474 mg, 1475 mg, 1476 mg, 1477 mg, 1478 mg, 1479 mg, 1480 mg, 1481 mg, 1482 mg, 1483 mg, 1484 mg, 1485 mg, 1486 mg, 1487 mg, 1488 mg, 1489 mg, 1490 mg, 1491 mg, 1492 mg, 1493 mg, 1494 mg, 1495 mg, 1496 mg, 1497 mg, 1498 mg, 1499 mg, or 1500 mg CBD or a pharmaceutically acceptable salt or derivative thereof per dose).

Thus, in an embodiment, the pharmaceutical composition comprises from about 1 mg to about 1500 mg CBD or a pharmaceutically acceptable salt or derivative thereof per dose, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, preferably about 100 mg, preferably about 101 mg, preferably about 102 mg, preferably about 103 mg, preferably about 104 mg, preferably about 105 mg, preferably about 106 mg, preferably about 107 mg, preferably about 108 mg, preferably about 109 mg, preferably about 110 mg, preferably about 111 mg, preferably about 112 mg, preferably about 113 mg, preferably about 114 mg, preferably about 115 mg, preferably about 116 mg, preferably about 117 mg, preferably about 118 mg, preferably about 119 mg, preferably about 120 mg, preferably about 121 mg, preferably about 122 mg, preferably about 123 mg, preferably about 124 mg, preferably about 125 mg, preferably about 126 mg, preferably about 127 mg, preferably about 128 mg, preferably about 129 mg, preferably about 130 mg, preferably about 131 mg, preferably about 132 mg, preferably about 131 mg, preferably about 134 mg, preferably about 135 mg, preferably about 136 mg, preferably about 137 mg, preferably about 138 mg, preferably about 139 mg, preferably about 140 mg, preferably about 141 mg, preferably about 142 mg, preferably about 143 mg, preferably about 144 mg, preferably about 145 mg, preferably about 146 mg, preferably about 147 mg, preferably about 148 mg, preferably about 149 mg, preferably about 150 mg, preferably about 151 mg, preferably about 152 mg, preferably about 153 mg, preferably about 154 mg, preferably about 155 mg, preferably about 156 mg, preferably about 157 mg, preferably about 158 mg, preferably about 159 mg, preferably about 160 mg, preferably about 161 mg, preferably about 162 mg, preferably about 163 mg, preferably about 164 mg, preferably about 165 mg, preferably about 166 mg, preferably about 167 mg, preferably about 168 mg, preferably about 169 mg, preferably about 170 mg, preferably about 171 mg, preferably about 172 mg, preferably about 173 mg, preferably about 174 mg, preferably about 175 mg, preferably about 176 mg, preferably about 177 mg, preferably about 178 mg, preferably about 179 mg, preferably about 180 mg, preferably about 181 mg, preferably about 182 mg, preferably about 183 mg, preferably about 184 mg, preferably about 185 mg, preferably about 186 mg, preferably about 187 mg, preferably about 188 mg, preferably about 189 mg, preferably about 190 mg, preferably about 191 mg, preferably about 192 mg, preferably about 193 mg, preferably about 194 mg, preferably about 195 mg, preferably about 196 mg, preferably about 197 mg, preferably about 198 mg, preferably about 199 mg, preferably about 200 mg, preferably about 201 mg, preferably about 202 mg, preferably about 203 mg, preferably about 204 mg, preferably about 205 mg, preferably about 206 mg, preferably about 207 mg, preferably about 208 mg, preferably about 209 mg, preferably about 210 mg, preferably about 211 mg, preferably about 212 mg, preferably about 213 mg, preferably about 214 mg, preferably about 215 mg, preferably about 216 mg, preferably about 217 mg, preferably about 218 mg, preferably about 219 mg, preferably about 220 mg, preferably about 221 mg, preferably about 222 mg, preferably about 223 mg, preferably about 224 mg, preferably about 225 mg, preferably about 226 mg, preferably about 227 mg, preferably about 228 mg, preferably about 229 mg, preferably about 230 mg, preferably about 231 mg, preferably about 232 mg, preferably about 233 mg, preferably about 234 mg, preferably about 235 mg, preferably about 236 mg, preferably about 237 mg, preferably about 238 mg, preferably about 239 mg, preferably about 240 mg, preferably about 241 mg, preferably about 242 mg, preferably about 243 mg, preferably about 244 mg, preferably about 245 mg, preferably about 246 mg, preferably about 247 mg, preferably about 248 mg, preferably about 249 mg, preferably about 250 mg, preferably about 251 mg, preferably about 252 mg, preferably about 253 mg, preferably about 254 mg, preferably about 255 mg, preferably about 256 mg, preferably about 257 mg, preferably about 258 mg, preferably about 259 mg, preferably about 260 mg, preferably about 261 mg, preferably about 262 mg, preferably about 263 mg, preferably about 264 mg, preferably about 265 mg, preferably about 266 mg, preferably about 267 mg, preferably about 268 mg, preferably about 269 mg, preferably about 270 mg, preferably about 271 mg, preferably about 272 mg, preferably about 273 mg, preferably about 274 mg, preferably about 275 mg, preferably about 276 mg, preferably about 277 mg, preferably about 278 mg, preferably about 279 mg, preferably about 280 mg, preferably about 281 mg, preferably about 282 mg, preferably about 283 mg, preferably about 284 mg, preferably about 285 mg, preferably about 286 mg, preferably about 287 mg, preferably about 288 mg, preferably about 289 mg, preferably about 290 mg, preferably about 291 mg, preferably about 292 mg, preferably about 293 mg, preferably about 294 mg, preferably about 295 mg, preferably about 296 mg, preferably about 297 mg, preferably about 298 mg, preferably about 299 mg, preferably about 300 mg, preferably about 301 mg, preferably about 302 mg, preferably about 303 mg, preferably about 304 mg, preferably about 305 mg, preferably about 306 mg, preferably about 307 mg, preferably about 308 mg, preferably about 309 mg, preferably about 310 mg, preferably about 311 mg, preferably about 312 mg, preferably about 313 mg, preferably about 314 mg, preferably about 315 mg, preferably about 316 mg, preferably about 317 mg, preferably about 318 mg, preferably about 319 mg, preferably about 320 mg, preferably about 321 mg, preferably about 322 mg, preferably about 323 mg, preferably about 324 mg, preferably about 325 mg, preferably about 326 mg, preferably about 327 mg, preferably about 328 mg, preferably about 329 mg, preferably about 330 mg, preferably about 331 mg, preferably about 332 mg, preferably about 333 mg, preferably about 334 mg, preferably about 335 mg, preferably about 336 mg, preferably about 337 mg, preferably about 338 mg, preferably about 339 mg, preferably about 340 mg, preferably about 341 mg, preferably about 342 mg, preferably about 343 mg, preferably about 344 mg, preferably about 345 mg, preferably about 346 mg, preferably about 347 mg, preferably about 348 mg, preferably about 349 mg, preferably about 350 mg, preferably about 351 mg, preferably about 352 mg, preferably about 353 mg, preferably about 354 mg, preferably about 355 mg, preferably about 356 mg, preferably about 357 mg, preferably about 358 mg, preferably about 359 mg, preferably about 360 mg, preferably about 361 mg, preferably about 362 mg, preferably about 363 mg, preferably about 364 mg, preferably about 365 mg, preferably about 366 mg, preferably about 367 mg, preferably about 368 mg, preferably about 369 mg, preferably about 370 mg, preferably about 371 mg, preferably about 372 mg, preferably about 373 mg, preferably about 374 mg, preferably about 375 mg, preferably about 376 mg, preferably about 377 mg, preferably about 378 mg, preferably about 379 mg, preferably about 380 mg, preferably about 381 mg, preferably about 382 mg, preferably about 383 mg, preferably about 384 mg, preferably about 385 mg, preferably about 386 mg, preferably about 387 mg, preferably about 388 mg, preferably about 389 mg, preferably about 390 mg, preferably about 391 mg, preferably about 392 mg, preferably about 393 mg, preferably about 394 mg, preferably about 395 mg, preferably about 396 mg, preferably about 397 mg, preferably about 398 mg, preferably about 399 mg, preferably about 400 mg, preferably about 401 mg, preferably about 402 mg, preferably about 403 mg, preferably about 404 mg, preferably about 405 mg, preferably about 406 mg, preferably about 407 mg, preferably about 408 mg, preferably about 409 mg, preferably about 410 mg, preferably about 411 mg, preferably about 412 mg, preferably about 413 mg, preferably about 414 mg, preferably about 415 mg, preferably about 416 mg, preferably about 417 mg, preferably about 418 mg, preferably about 419 mg, preferably about 420 mg, preferably about 421 mg, preferably about 422 mg, preferably about 423 mg, preferably about 424 mg, preferably about 425 mg, preferably about 426 mg, preferably about 427 mg, preferably about 428 mg, preferably about 429 mg, preferably about 430 mg, preferably about 431 mg, preferably about 432 mg, preferably about 433 mg, preferably about 434 mg, preferably about 435 mg, preferably about 436 mg, preferably about 437 mg, preferably about 438 mg, preferably about 439 mg, preferably about 440 mg, preferably about 441 mg, preferably about 442 mg, preferably about 443 mg, preferably about 444 mg, preferably about 445 mg, preferably about 446 mg, preferably about 447 mg, preferably about 448 mg, preferably about 449 mg, preferably about 450 mg, preferably about 451 mg, preferably about 452 mg, preferably about 453 mg, preferably about 454 mg, preferably about 455 mg, preferably about 456 mg, preferably about 457 mg, preferably about 458 mg, preferably about 459 mg, preferably about 460 mg, preferably about 461 mg, preferably about 462 mg, preferably about 463 mg, preferably about 464 mg, preferably about 465 mg, preferably about 466 mg, preferably about 467 mg, preferably about 468 mg, preferably about 469 mg, preferably about 470 mg, preferably about 471 mg, preferably about 472 mg, preferably about 473 mg, preferably about 474 mg, preferably about 475 mg, preferably about 476 mg, preferably about 477 mg, preferably about 478 mg, preferably about 479 mg, preferably about 480 mg, preferably about 481 mg, preferably about 482 mg, preferably about 483 mg, preferably about 484 mg, preferably about 485 mg, preferably about 486 mg, preferably about 487 mg, preferably about 488 mg, preferably about 489 mg, preferably about 490 mg, preferably about 491 mg, preferably about 492 mg, preferably about 493 mg, preferably about 494 mg, preferably about 495 mg, preferably about 496 mg, preferably about 497 mg, preferably about 498 mg, preferably about 499 mg, preferably about 500 mg, preferably about 501 mg, preferably about 502 mg, preferably about 503 mg, preferably about 504 mg, preferably about 505 mg, preferably about 506 mg, preferably about 507 mg, preferably about 508 mg, preferably about 509 mg, preferably about 510 mg, preferably about 511 mg, preferably about 512 mg, preferably about 513 mg, preferably about 514 mg, preferably about 515 mg, preferably about 516 mg, preferably about 517 mg, preferably about 518 mg, preferably about 519 mg, preferably about 520 mg, preferably about 521 mg, preferably about 522 mg, preferably about 523 mg, preferably about 524 mg, preferably about 525 mg, preferably about 526 mg, preferably about 527 mg, preferably about 528 mg, preferably about 529 mg, preferably about 530 mg, preferably about 531 mg, preferably about 532 mg, preferably about 533 mg, preferably about 534 mg, preferably about 535 mg, preferably about 536 mg, preferably about 537 mg, preferably about 538 mg, preferably about 539 mg, preferably about 540 mg, preferably about 541 mg, preferably about 542 mg, preferably about 543 mg, preferably about 544 mg, preferably about 545 mg, preferably about 546 mg, preferably about 547 mg, preferably about 548 mg, preferably about 549 mg, preferably about 550 mg, preferably about 551 mg, preferably about 552 mg, preferably about 553 mg, preferably about 554 mg, preferably about 555 mg, preferably about 556 mg, preferably about 557 mg, preferably about 558 mg, preferably about 559 mg, preferably about 560 mg, preferably about 561 mg, preferably about 562 mg, preferably about 563 mg, preferably about 564 mg, preferably about 565 mg, preferably about 566 mg, preferably about 567 mg, preferably about 568 mg, preferably about 569 mg, preferably about 570 mg, preferably about 571 mg, preferably about 572 mg, preferably about 573 mg, preferably about 574 mg, preferably about 575 mg, preferably about 576 mg, preferably about 577 mg, preferably about 578 mg, preferably about 579 mg, preferably about 580 mg, preferably about 581 mg, preferably about 582 mg, preferably about 583 mg, preferably about 584 mg, preferably about 585 mg, preferably about 586 mg, preferably about 587 mg, preferably about 588 mg, preferably about 589 mg, preferably about 590 mg, preferably about 591 mg, preferably about 592 mg, preferably about 593 mg, preferably about 594 mg, preferably about 595 mg, preferably about 596 mg, preferably about 597 mg, preferably about 598 mg, preferably about 599 mg, preferably about 600 mg, preferably about 601 mg, preferably about 602 mg, preferably about 603 mg, preferably about 604 mg, preferably about 605 mg, preferably about 606 mg, preferably about 607 mg, preferably about 608 mg, preferably about 609 mg, preferably about 610 mg, preferably about 611 mg, preferably about 612 mg, preferably about 613 mg, preferably about 614 mg, preferably about 615 mg, preferably about 616 mg, preferably about 617 mg, preferably about 618 mg, preferably about 619 mg, preferably about 620 mg, preferably about 621 mg, preferably about 622 mg, preferably about 623 mg, preferably about 624 mg, preferably about 625 mg, preferably about 626 mg, preferably about 627 mg, preferably about 628 mg, preferably about 629 mg, preferably about 630 mg, preferably about 631 mg, preferably about 632 mg, preferably about 633 mg, preferably about 634 mg, preferably about 635 mg, preferably about 636 mg, preferably about 637 mg, preferably about 638 mg, preferably about 639 mg, preferably about 640 mg, preferably about 641 mg, preferably about 642 mg, preferably about 643 mg, preferably about 644 mg, preferably about 645 mg, preferably about 646 mg, preferably about 647 mg, preferably about 648 mg, preferably about 649 mg, preferably about 650 mg, preferably about 651 mg, preferably about 652 mg, preferably about 653 mg, preferably about 654 mg, preferably about 655 mg, preferably about 656 mg, preferably about 657 mg, preferably about 658 mg, preferably about 659 mg, preferably about 660 mg, preferably about 661 mg, preferably about 662 mg, preferably about 663 mg, preferably about 664 mg, preferably about 665 mg, preferably about 666 mg, preferably about 667 mg, preferably about 668 mg, preferably about 669 mg, preferably about 670 mg, preferably about 671 mg, preferably about 672 mg, preferably about 673 mg, preferably about 674 mg, preferably about 675 mg, preferably about 676 mg, preferably about 677 mg, preferably about 678 mg, preferably about 679 mg, preferably about 680 mg, preferably about 681 mg, preferably about 682 mg, preferably about 683 mg, preferably about 684 mg, preferably about 685 mg, preferably about 686 mg, preferably about 687 mg, preferably about 688 mg, preferably about 689 mg, preferably about 690 mg, preferably about 691 mg, preferably about 692 mg, preferably about 693 mg, preferably about 694 mg, preferably about 695 mg, preferably about 696 mg, preferably about 697 mg, preferably about 698 mg, preferably about 699 mg, preferably about 700 mg, preferably about 701 mg, preferably about 702 mg, preferably about 703 mg, preferably about 704 mg, preferably about 705 mg, preferably about 706 mg, preferably about 707 mg, preferably about 708 mg, preferably about 709 mg, preferably about 710 mg, preferably about 711 mg, preferably about 712 mg, preferably about 713 mg, preferably about 714 mg, preferably about 715 mg, preferably about 716 mg, preferably about 717 mg, preferably about 718 mg, preferably about 719 mg, preferably about 720 mg, preferably about 721 mg, preferably about 722 mg, preferably about 723 mg, preferably about 724 mg, preferably about 725 mg, preferably about 726 mg, preferably about 727 mg, preferably about 728 mg, preferably about 729 mg, preferably about 730 mg, preferably about 731 mg, preferably about 732 mg, preferably about 733 mg, preferably about 734 mg, preferably about 735 mg, preferably about 736 mg, preferably about 737 mg, preferably about 738 mg, preferably about 739 mg, preferably about 740 mg, preferably about 741 mg, preferably about 742 mg, preferably about 743 mg, preferably about 744 mg, preferably about 745 mg, preferably about 746 mg, preferably about 747 mg, preferably about 748 mg, preferably about 749 mg, preferably about 750 mg, preferably about 751 mg, preferably about 752 mg, preferably about 753 mg, preferably about 754 mg, preferably about 755 mg, preferably about 756 mg, preferably about 757 mg, preferably about 758 mg, preferably about 759 mg, preferably about 760 mg, preferably about 761 mg, preferably about 762 mg, preferably about 763 mg, preferably about 764 mg, preferably about 765 mg, preferably about 766 mg, preferably about 767 mg, preferably about 768 mg, preferably about 769 mg, preferably about 770 mg, preferably about 771 mg, preferably about 772 mg, preferably about 773 mg, preferably about 774 mg, preferably about 775 mg, preferably about 776 mg, preferably about 777 mg, preferably about 778 mg, preferably about 779 mg, preferably about 780 mg, preferably about 781 mg, preferably about 782 mg, preferably about 783 mg, preferably about 784 mg, preferably about 785 mg, preferably about 786 mg, preferably about 787 mg, preferably about 788 mg, preferably about 789 mg, preferably about 790 mg, preferably about 791 mg, preferably about 792 mg, preferably about 793 mg, preferably about 794 mg, preferably about 795 mg, preferably about 796 mg, preferably about 797 mg, preferably about 798 mg, preferably about 799 mg, preferably about 800 mg, preferably about 801 mg, preferably about 802 mg, preferably about 803 mg, preferably about 804 mg, preferably about 805 mg, preferably about 806 mg, preferably about 807 mg, preferably about 808 mg, preferably about 809 mg, preferably about 810 mg, preferably about 811 mg, preferably about 812 mg, preferably about 813 mg, preferably about 814 mg, preferably about 815 mg, preferably about 816 mg, preferably about 817 mg, preferably about 818 mg, preferably about 819 mg, preferably about 820 mg, preferably about 821 mg, preferably about 822 mg, preferably about 823 mg, preferably about 824 mg, preferably about 825 mg, preferably about 826 mg, preferably about 827 mg, preferably about 828 mg, preferably about 829 mg, preferably about 830 mg, preferably about 831 mg, preferably about 832 mg, preferably about 833 mg, preferably about 834 mg, preferably about 835 mg, preferably about 836 mg, preferably about 837 mg, preferably about 838 mg, preferably about 839 mg, preferably about 840 mg, preferably about 841 mg, preferably about 842 mg, preferably about 843 mg, preferably about 844 mg, preferably about 845 mg, preferably about 846 mg, preferably about 847 mg, preferably about 848 mg, preferably about 849 mg, preferably about 850 mg, preferably about 851 mg, preferably about 852 mg, preferably about 853 mg, preferably about 854 mg, preferably about 855 mg, preferably about 856 mg, preferably about 857 mg, preferably about 858 mg, preferably about 859 mg, preferably about 860 mg, preferably about 861 mg, preferably about 862 mg, preferably about 863 mg, preferably about 864 mg, preferably about 865 mg, preferably about 866 mg, preferably about 867 mg, preferably about 868 mg, preferably about 869 mg, preferably about 870 mg, preferably about 871 mg, preferably about 872 mg, preferably about 873 mg, preferably about 874 mg, preferably about 875 mg, preferably about 876 mg, preferably about 877 mg, preferably about 878 mg, preferably about 879 mg, preferably about 880 mg, preferably about 881 mg, preferably about 882 mg, preferably about 883 mg, preferably about 884 mg, preferably about 885 mg, preferably about 886 mg, preferably about 887 mg, preferably about 888 mg, preferably about 889 mg, preferably about 890 mg, preferably about 891 mg, preferably about 892 mg, preferably about 893 mg, preferably about 894 mg, preferably about 895 mg, preferably about 896 mg, preferably about 897 mg, preferably about 898 mg, preferably about 899 mg, preferably about 900 mg, preferably about 901 mg, preferably about 902 mg, preferably about 903 mg, preferably about 904 mg, preferably about 905 mg, preferably about 906 mg, preferably about 907 mg, preferably about 908 mg, preferably about 909 mg, preferably about 910 mg, preferably about 911 mg, preferably about 912 mg, preferably about 913 mg, preferably about 914 mg, preferably about 915 mg, preferably about 916 mg, preferably about 917 mg, preferably about 918 mg, preferably about 919 mg, preferably about 920 mg, preferably about 921 mg, preferably about 922 mg, preferably about 923 mg, preferably about 924 mg, preferably about 925 mg, preferably about 926 mg, preferably about 927 mg, preferably about 928 mg, preferably about 929 mg, preferably about 930 mg, preferably about 931 mg, preferably about 932 mg, preferably about 933 mg, preferably about 934 mg, preferably about 935 mg, preferably about 936 mg, preferably about 937 mg, preferably about 938 mg, preferably about 939 mg, preferably about 940 mg, preferably about 941 mg, preferably about 942 mg, preferably about 943 mg, preferably about 944 mg, preferably about 945 mg, preferably about 946 mg, preferably about 947 mg, preferably about 948 mg, preferably about 949 mg, preferably about 950 mg, preferably about 951 mg, preferably about 952 mg, preferably about 953 mg, preferably about 954 mg, preferably about 955 mg, preferably about 956 mg, preferably about 957 mg, preferably about 958 mg, preferably about 959 mg, preferably about 960 mg, preferably about 961 mg, preferably about 962 mg, preferably about 963 mg, preferably about 964 mg, preferably about 965 mg, preferably about 966 mg, preferably about 967 mg, preferably about 968 mg, preferably about 969 mg, preferably about 970 mg, preferably about 971 mg, preferably about 972 mg, preferably about 973 mg, preferably about 974 mg, preferably about 975 mg, preferably about 976 mg, preferably about 977 mg, preferably about 978 mg, preferably about 979 mg, preferably about 980 mg, preferably about 981 mg, preferably about 982 mg, preferably about 983 mg, preferably about 984 mg, preferably about 985 mg, preferably about 986 mg, preferably about 987 mg, preferably about 988 mg, preferably about 989 mg, preferably about 990 mg, preferably about 991 mg, preferably about 992 mg, preferably about 993 mg, preferably about 994 mg, preferably about 995 mg, preferably about 996 mg, preferably about 997 mg, preferably about 998 mg, preferably about 999 mg, preferably about 1000 mg, preferably about 1001 mg, preferably about 1002 mg, preferably about 1003 mg, preferably about 1004 mg, preferably about 1005 mg, preferably about 1006 mg, preferably about 1007 mg, preferably about 1008 mg, preferably about 1009 mg, preferably about 1010 mg, preferably about 1011 mg, preferably about 1012 mg, preferably about 1013 mg, preferably about 1014 mg, preferably about 1015 mg, preferably about 1016 mg, preferably about 1017 mg, preferably about 1018 mg, preferably about 1019 mg, preferably about 1020 mg, preferably about 1021 mg, preferably about 1022 mg, preferably about 1023 mg, preferably about 1024 mg, preferably about 1025 mg, preferably about 1026 mg, preferably about 1027 mg, preferably about 1028 mg, preferably about 1029 mg, preferably about 1030 mg, preferably about 1031 mg, preferably about 1032 mg, preferably about 1033 mg, preferably about 1034 mg, preferably about 1035 mg, preferably about 1036 mg, preferably about 1037 mg, preferably about 1038 mg, preferably about 1039 mg, preferably about 1040 mg, preferably about 1041 mg, preferably about 1042 mg, preferably about 1043 mg, preferably about 1044 mg, preferably about 1045 mg, preferably about 1046 mg, preferably about 1047 mg, preferably about 1048 mg, preferably about 1049 mg, preferably about 1050 mg, preferably about 1051 mg, preferably about 1052 mg, preferably about 1053 mg, preferably about 1054 mg, preferably about 1055 mg, preferably about 1056 mg, preferably about 1057 mg, preferably about 1058 mg, preferably about 1059 mg, preferably about 1060 mg, preferably about 1061 mg, preferably about 1062 mg, preferably about 1063 mg, preferably about 1064 mg, preferably about 1065 mg, preferably about 1066 mg, preferably about 1067 mg, preferably about 1068 mg, preferably about 1069 mg, preferably about 1070 mg, preferably about 1071 mg, preferably about 1072 mg, preferably about 1073 mg, preferably about 1074 mg, preferably about 1075 mg, preferably about 1076 mg, preferably about 1077 mg, preferably about 1078 mg, preferably about 1079 mg, preferably about 1080 mg, preferably about 1081 mg, preferably about 1082 mg, preferably about 1083 mg, preferably about 1084 mg, preferably about 1085 mg, preferably about 1086 mg, preferably about 1087 mg, preferably about 1088 mg, preferably about 1089 mg, preferably about 1090 mg, preferably about 1091 mg, preferably about 1092 mg, preferably about 1093 mg, preferably about 1094 mg, preferably about 1095 mg, preferably about 1096 mg, preferably about 1097 mg, preferably about 1098 mg, preferably about 1099 mg, preferably about 1100 mg, preferably about 1101 mg, preferably about 1102 mg, preferably about 1103 mg, preferably about 1104 mg, preferably about 1105 mg, preferably about 1106 mg, preferably about 1107 mg, preferably about 1108 mg, preferably about 1109 mg, preferably about 1110 mg, preferably about 1111 mg, preferably about 1112 mg, preferably about 1113 mg, preferably about 1114 mg, preferably about 1115 mg, preferably about 1116 mg, preferably about 1117 mg, preferably about 1118 mg, preferably about 1119 mg, preferably about 1120 mg, preferably about 1121 mg, preferably about 1122 mg, preferably about 1123 mg, preferably about 1124 mg, preferably about 1125 mg, preferably about 1126 mg, preferably about 1127 mg, preferably about 1128 mg, preferably about 1129 mg, preferably about 1130 mg, preferably about 1131 mg, preferably about 1132 mg, preferably about 1133 mg, preferably about 1134 mg, preferably about 1135 mg, preferably about 1136 mg, preferably about 1137 mg, preferably about 1138 mg, preferably about 1139 mg, preferably about 1140 mg, preferably about 1141 mg, preferably about 1142 mg, preferably about 1143 mg, preferably about 1144 mg, preferably about 1145 mg, preferably about 1146 mg, preferably about 1147 mg, preferably about 1148 mg, preferably about 1149 mg, preferably about 1150 mg, preferably about 1151 mg, preferably about 1152 mg, preferably about 1153 mg, preferably about 1154 mg, preferably about 1155 mg, preferably about 1156 mg, preferably about 1157 mg, preferably about 1158 mg, preferably about 1159 mg, preferably about 1160 mg, preferably about 1161 mg, preferably about 1162 mg, preferably about 1163 mg, preferably about 1164 mg, preferably about 1165 mg, preferably about 1166 mg, preferably about 1167 mg, preferably about 1168 mg, preferably about 1169 mg, preferably about 1170 mg, preferably about 1171 mg, preferably about 1172 mg, preferably about 1173 mg, preferably about 1174 mg, preferably about 1175 mg, preferably about 1176 mg, preferably about 1177 mg, preferably about 1178 mg, preferably about 1179 mg, preferably about 1180 mg, preferably about 1181 mg, preferably about 1182 mg, preferably about 1183 mg, preferably about 1184 mg, preferably about 1185 mg, preferably about 1186 mg, preferably about 1187 mg, preferably about 1188 mg, preferably about 1189 mg, preferably about 1190 mg, preferably about 1191 mg, preferably about 1192 mg, preferably about 1193 mg, preferably about 1194 mg, preferably about 1195 mg, preferably about 1196 mg, preferably about 1197 mg, preferably about 1198 mg, preferably about 1199 mg, preferably about 1200 mg, preferably about 1201 mg, preferably about 1202 mg, preferably about 1203 mg, preferably about 1204 mg, preferably about 1205 mg, preferably about 1206 mg, preferably about 1207 mg, preferably about 1208 mg, preferably about 1209 mg, preferably about 1210 mg, preferably about 1211 mg, preferably about 1212 mg, preferably about 1213 mg, preferably about 1214 mg, preferably about 1215 mg, preferably about 1216 mg, preferably about 1217 mg, preferably about 1218 mg, preferably about 1219 mg, preferably about 1220 mg, preferably about 1221 mg, preferably about 1222 mg, preferably about 1223 mg, preferably about 1224 mg, preferably about 1225 mg, preferably about 1226 mg, preferably about 1227 mg, preferably about 1228 mg, preferably about 1229 mg, preferably about 1230 mg, preferably about 1231 mg, preferably about 1232 mg, preferably about 1233 mg, preferably about 1234 mg, preferably about 1235 mg, preferably about 1236 mg, preferably about 1237 mg, preferably about 1238 mg, preferably about 1239 mg, preferably about 1240 mg, preferably about 1241 mg, preferably about 1242 mg, preferably about 1243 mg, preferably about 1244 mg, preferably about 1245 mg, preferably about 1246 mg, preferably about 1247 mg, preferably about 1248 mg, preferably about 1249 mg, preferably about 1250 mg, preferably about 1251 mg, preferably about 1252 mg, preferably about 1253 mg, preferably about 1254 mg, preferably about 1255 mg, preferably about 1256 mg, preferably about 1257 mg, preferably about 1258 mg, preferably about 1259 mg, preferably about 1260 mg, preferably about 1261 mg, preferably about 1262 mg, preferably about 1263 mg, preferably about 1264 mg, preferably about 1265 mg, preferably about 1266 mg, preferably about 1267 mg, preferably about 1268 mg, preferably about 1269 mg, preferably about 1270 mg, preferably about 1271 mg, preferably about 1272 mg, preferably about 1273 mg, preferably about 1274 mg, preferably about 1275 mg, preferably about 1276 mg, preferably about 1277 mg, preferably about 1278 mg, preferably about 1279 mg, preferably about 1280 mg, preferably about 1281 mg, preferably about 1282 mg, preferably about 1283 mg, preferably about 1284 mg, preferably about 1285 mg, preferably about 1286 mg, preferably about 1287 mg, preferably about 1288 mg, preferably about 1289 mg, preferably about 1290 mg, preferably about 1291 mg, preferably about 1292 mg, preferably about 1293 mg, preferably about 1294 mg, preferably about 1295 mg, preferably about 1296 mg, preferably about 1297 mg, preferably about 1298 mg, preferably about 1299 mg, preferably about 1300 mg, preferably about 1301 mg, preferably about 1302 mg, preferably about 1303 mg, preferably about 1304 mg, preferably about 1305 mg, preferably about 1306 mg, preferably about 1307 mg, preferably about 1308 mg, preferably about 1309 mg, preferably about 1310 mg, preferably about 1311 mg, preferably about 1312 mg, preferably about 1313 mg, preferably about 1314 mg, preferably about 1315 mg, preferably about 1316 mg, preferably about 1317 mg, preferably about 1318 mg, preferably about 1319 mg, preferably about 1320 mg, preferably about 1321 mg, preferably about 1322 mg, preferably about 1323 mg, preferably about 1324 mg, preferably about 1325 mg, preferably about 1326 mg, preferably about 1327 mg, preferably about 1328 mg, preferably about 1329 mg, preferably about 1330 mg, preferably about 1331 mg, preferably about 1332 mg, preferably about 1333 mg, preferably about 1334 mg, preferably about 1335 mg, preferably about 1336 mg, preferably about 1337 mg, preferably about 1338 mg, preferably about 1339 mg, preferably about 1340 mg, preferably about 1341 mg, preferably about 1342 mg, preferably about 1343 mg, preferably about 1344 mg, preferably about 1345 mg, preferably about 1346 mg, preferably about 1347 mg, preferably about 1348 mg, preferably about 1349 mg, preferably about 1350 mg, preferably about 1351 mg, preferably about 1352 mg, preferably about 1353 mg, preferably about 1354 mg, preferably about 1355 mg, preferably about 1356 mg, preferably about 1357 mg, preferably about 1358 mg, preferably about 1359 mg, preferably about 1360 mg, preferably about 1361 mg, preferably about 1362 mg, preferably about 1363 mg, preferably about 1364 mg, preferably about 1365 mg, preferably about 1366 mg, preferably about 1367 mg, preferably about 1368 mg, preferably about 1369 mg, preferably about 1370 mg, preferably about 1371 mg, preferably about 1372 mg, preferably about 1373 mg, preferably about 1374 mg, preferably about 1375 mg, preferably about 1376 mg, preferably about 1377 mg, preferably about 1378 mg, preferably about 1379 mg, preferably about 1380 mg, preferably about 1381 mg, preferably about 1382 mg, preferably about 1383 mg, preferably about 1384 mg, preferably about 1385 mg, preferably about 1386 mg, preferably about 1387 mg, preferably about 1388 mg, preferably about 1389 mg, preferably about 1390 mg, preferably about 1391 mg, preferably about 1392 mg, preferably about 1393 mg, preferably about 1394 mg, preferably about 1395 mg, preferably about 1396 mg, preferably about 1397 mg, preferably about 1398 mg, preferably about 1399 mg, preferably about 1400 mg, preferably about 1401 mg, preferably about 1402 mg, preferably about 1403 mg, preferably about 1404 mg, preferably about 1405 mg, preferably about 1406 mg, preferably about 1407 mg, preferably about 1408 mg, preferably about 1409 mg, preferably about 1410 mg, preferably about 1411 mg, preferably about 1412 mg, preferably about 1413 mg, preferably about 1414 mg, preferably about 1415 mg, preferably about 1416 mg, preferably about 1417 mg, preferably about 1418 mg, preferably about 1419 mg, preferably about 1420 mg, preferably about 1421 mg, preferably about 1422 mg, preferably about 1423 mg, preferably about 1424 mg, preferably about 1425 mg, preferably about 1426 mg, preferably about 1427 mg, preferably about 1428 mg, preferably about 1429 mg, preferably about 1430 mg, preferably about 1431 mg, preferably about 1432 mg, preferably about 1433 mg, preferably about 1434 mg, preferably about 1435 mg, preferably about 1436 mg, preferably about 1437 mg, preferably about 1438 mg, preferably about 1439 mg, preferably about 1440 mg, preferably about 1441 mg, preferably about 1442 mg, preferably about 1443 mg, preferably about 1444 mg, preferably about 1445 mg, preferably about 1446 mg, preferably about 1447 mg, preferably about 1448 mg, preferably about 1449 mg, preferably about 1450 mg, preferably about 1451 mg, preferably about 1452 mg, preferably about 1453 mg, preferably about 1454 mg, preferably about 1455 mg, preferably about 1456 mg, preferably about 1457 mg, preferably about 1458 mg, preferably about 1459 mg, preferably about 1460 mg, preferably about 1461 mg, preferably about 1462 mg, preferably about 1463 mg, preferably about 1464 mg, preferably about 1465 mg, preferably about 1466 mg, preferably about 1467 mg, preferably about 1468 mg, preferably about 1469 mg, preferably about 1470 mg, preferably about 1471 mg, preferably about 1472 mg, preferably about 1473 mg, preferably about 1474 mg, preferably about 1475 mg, preferably about 1476 mg, preferably about 1477 mg, preferably about 1478 mg, preferably about 1479 mg, preferably about 1480 mg, preferably about 1481 mg, preferably about 1482 mg, preferably about 1483 mg, preferably about 1484 mg, preferably about 1485 mg, preferably about 1486 mg, preferably about 1487 mg, preferably about 1488 mg, preferably about 1489 mg, preferably about 1490 mg, preferably about 1491 mg, preferably about 1492 mg, preferably about 1493 mg, preferably about 1494 mg, preferably about 1495 mg, preferably about 1496 mg, preferably about 1497 mg, preferably about 1498 mg, preferably about 1499 mg, or more preferably about 1500 mg CBD or a pharmaceutically acceptable salt or derivative thereof per dose.

In an embodiment, the pharmaceutical composition comprises from about 20 mg to about 50 mg CBD or a pharmaceutically acceptable salt or derivative thereof per dose.

In an embodiment, the pharmaceutical composition comprises from about 50 mg to about 500 mg CBD or a pharmaceutically acceptable salt or derivative thereof per dose.

In an embodiment, the pharmaceutical composition comprises from about 500 mg to about 1500 mg CBD or a pharmaceutically acceptable salt or derivative thereof per dose.

In an embodiment, the pharmaceutical composition comprises from about 1 mg to about 50 mg of the acetylcholinesterase inhibitor per dose (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg of the acetylcholinesterase inhibitor per dose).

Thus, in an embodiment, the pharmaceutical composition comprises from about 1 mg to about 50 mg of the acetylcholinesterase inhibitor per dose, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, or more preferably about 50 mg of the acetylcholinesterase inhibitor per dose.

In an embodiment, the pharmaceutical composition comprises from about 5 mg to about 10 mg of the acetylcholinesterase inhibitor per dose.

In an embodiment, the pharmaceutical composition comprises from about 10 mg to about 25 mg of the acetylcholinesterase inhibitor per dose.

In an embodiment, the pharmaceutical composition further comprises one or more pharmaceutically acceptable carriers, diluents and excipients.

Suitable pharmaceutically acceptable carriers, diluents or excipients would be known to persons skilled in the art, illustrative examples of which include inert diluents (e.g., calcium carbonate, lactose, calcium phosphate or sodium phosphate), granulating and disintegrating agents (e.g., corn starch or alginic acid), binding agents (e.g., starch, gelatin or acacia), lubricating agents (e.g., magnesium stearate, stearic acid or talc) and material to delay disintegration and absorption in the gastrointestinal tract and thereby provide a sustained action over a longer period (e.g., glyceryl monostearate or glyceryl distearate). Coating may also be performed using techniques described in the U.S. Pat. Nos. 4,256,108, 4,160,452, and 4,265,874 to form osmotic therapeutic tablets for control release.

The pharmaceutical composition disclosed herein may be for administration by any suitable route that allows for delivery of the CBD or a pharmaceutically acceptable salt or derivative thereof and the acetylcholinesterase inhibitor to a subject at a therapeutically effective amount, as described herein. Suitable administrative forms will be known to persons skilled in the art, illustrative examples of which include enteral administration (e.g., oral and rectal), parenteral administration, typically injectable or micro-injectable forms (e.g., intramuscular, subcutaneous, intravenous, epidural, intra-articular, intraperitoneal, intracisternal or intrathecal) and topical (transdermal or transmucosal) administration (e.g., buccal, sublingual, vaginal, intranasal or by inhalation).

In an embodiment, the pharmaceutical composition is for oral administration.

Oral administration of CBD has been demonstrated to be an effective administration route (reviewed by Millar et al., 2018, Frontiers in Pharmacology, 9: 1365). Similarly, oral administration of donepezil, rivastigmine and galantamine has been demonstrated to be effective for absorption with high bioavailability (see. e.g., Rogers and Friedhoff, 1998, British Journal of Clinical Pharmacology, 46(Suppl 1): 1-6; Hossain et al., 2002, Clinical Pharmacokinetics, 41(3): 225-234; and Huang and Fu, 2010, Current Clinical Pharmacology, 5(2): 115-24).

Suitable dosage forms for oral administration would be known to persons skilled in the art, illustrative examples of which include tablets, aqueous or oily suspensions, lozenges, troches, powders, granules, emulsions, capsules, liquids, syrups or elixirs.

In an embodiment, the CBD or a pharmaceutically acceptable salt or derivative thereof is in liquid, oil, tablet or capsule form. In another embodiment, the CBD or a pharmaceutically acceptable salt or derivative thereof is in liquid form.

In an embodiment, the acetylcholinesterase inhibitor is in liquid, oil, tablet or capsule form. In another embodiment, the acetylcholinesterase inhibitor is in tablet or capsule form.

Dosage forms for oral administration may comprise one or more agents selected from the group of sweetening agents, flavoring agents, coloring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Pharmaceutical compositions suitable for oral administration may be presented as discrete units (i.e., dosage forms), each containing a predetermined amount of each component of the pharmaceutical composition as a powder, tablet, capsule, granules, as a solution or a suspension in an aqueous liquid or non-aqueous liquid, or as an emulsion.

As described elsewhere herein, the pharmaceutical compositions may be formulated for administration as separate unit dosage forms for administration. The unit dosage form may be suitable for a capsule, tablet, oil or liquid solution.

In an embodiment, the composition further comprises one or more additional therapeutic agents selected from the group consisting of anti-inflammatory agents, antipsychotic agents, and analgesics.

In an embodiment, the composition further comprises the administration of one or more additional cannabinoids.

In an embodiment, the one or more additional cannabinoids are selected from the group consisting of CBG, CBN, CBDL, CBC and THC.

In an embodiment, the pharmaceutical composition comprises CBD or a pharmaceutically acceptable salt or derivative thereof, and donepezil or a pharmaceutically acceptable salt thereof.

In an embodiment, the pharmaceutical composition comprises CBD or a pharmaceutically acceptable salt or derivative thereof, and donepezil hydrochloride.

In an embodiment, the pharmaceutical composition comprises CBD or a pharmaceutically acceptable salt or derivative thereof, and rivastigmine or a pharmaceutically acceptable salt thereof.

In an embodiment, the pharmaceutical composition comprises CBD or a pharmaceutically acceptable salt or derivative thereof, and galantamine or a pharmaceutically acceptable salt thereof.

Pharmaceutical compositions disclosed herein may be prepared according to conventional methods well known in the pharmaceutical and nutraceutical industries, such as those described in Remington's Pharmaceutical Handbook (Mack Publishing Co., NY, USA).

In an aspect, there is provided a pharmaceutical composition comprising CBD or a pharmaceutically acceptable salt or derivative thereof, and an acetylcholinesterase inhibitor for use in the treatment or prevention of at least one neurodegenerative disorder and/or dementia.

In an aspect, there is provided a pharmaceutical composition comprising CBD or a pharmaceutically acceptable salt or derivative thereof, and an acetylcholinesterase inhibitor for use in the treatment or prevention of at least one neurodegenerative disorder.

In an aspect, there is provided a pharmaceutical composition comprising CBD or a pharmaceutically acceptable salt or derivative thereof, and an acetylcholinesterase inhibitor for use in the treatment or prevention of dementia.

In an aspect, there is provided a pharmaceutical composition comprising CBD or a pharmaceutically acceptable salt or derivative thereof, and an acetylcholinesterase inhibitor for use in the treatment or prevention of a disease associated with glutamate-mediated excitotoxicity.

### Kits

The present disclosure further contemplates a kit comprising CBD or a pharmaceutically acceptable salt or derivative thereof, and an acetylcholinesterase inhibitor for use in the treatment or prevention of at least one neurodegenerative disorder and/or dementia.

In an aspect, the present disclosure provides a kit comprising CBD or a pharmaceutically acceptable salt or derivative thereof, and an acetylcholinesterase inhibitor for use in the treatment or prevention of at least one neurodegenerative disorder.

In another aspect, the present disclosure provides a kit comprising CBD or a pharmaceutically acceptable salt or derivative thereof, and an acetylcholinesterase inhibitor for use in the treatment or prevention of dementia.

In another aspect, the present disclosure provides a kit comprising CBD or a pharmaceutically acceptable salt of derivative thereof, and an acetylcholinesterase inhibitor for use in the treatment or prevention of a disease associated with glutamate-mediated excitotoxicity.

In an embodiment, the kit comprises the CBD or a pharmaceutically acceptable salt or derivative thereof in a dosage form comprising from about 1 mg to about 1500 mg CBD or a pharmaceutically acceptable salt or derivative thereof per dose.

In an embodiment, the kit comprises the CBD or a pharmaceutically acceptable salt or derivative thereof in a dosage form comprising from about 20 mg to about 50 mg CBD or a pharmaceutically acceptable salt or derivative thereof per dose.

In an embodiment, the kit comprises the CBD or a pharmaceutically acceptable salt or derivative thereof in a dosage form comprising from about 50 mg to about 500 mg CBD or a pharmaceutically acceptable salt or derivative thereof per dose.

In an embodiment, the kit comprises the CBD or a pharmaceutically acceptable salt or derivative thereof in a dosage form comprising from about 500 mg to about 1500 mg CBD or a pharmaceutically acceptable salt or derivative thereof per dose.

In an embodiment, the kit comprises the acetylcholinesterase inhibitor in a dosage form comprising from about 5 mg to about 10 mg of the acetylcholinesterase inhibitor per dose.

In an embodiment, the kit comprises the acetylcholinesterase inhibitor in a dosage form comprising from about 10 mg to about 25 mg of the acetylcholinesterase inhibitor per dose.

All essential materials and reagents required for treating at least one neurodegenerative disorder and/or dementia or a disease associated with glutamate-mediated excitotoxicity in a subject may be assembled together in the kit. In an embodiment, the kit comprises two separate pharmaceutical compositions: one containing the CBD or a pharmaceutically acceptable salt or derivative thereof and the second containing the acetylcholinesterase inhibitor. In another embodiment, the kit comprises a containing or containing the separate compositions such as a divided bottle or a divided foil packet. Typically, the kit further comprises directions for the use of the components.

In an embodiment, the kit comprises the CBD or a pharmaceutically acceptable salt or derivative thereof and the acetylcholinesterase inhibitor in a blister pack. The term “blister pack” as used herein refers to a commonly used packaging of pharmaceutical unit dosage forms (e.g., tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. The tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via said opening.

In an embodiment, the kit further comprises a memory aid, e.g., in the form of numbers or a code next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent to persons skilled in the art.

The kit may also optionally include appropriate therapeutic agents to be administered in combination with the CBD or a pharmaceutically acceptable salt or derivative thereof and the acetylcholinesterase inhibitor, illustrative examples of which include anti-inflammatory agents, antipsychotic agents, and analgesics.

The present disclosure also contemplates a commercial package, also referred to as a “treatment pack” comprising the CBD or a pharmaceutically acceptable salt or derivative thereof, and the acetylcholinesterase inhibitor, together with instructions for use for the treatment or prevention of at least one neurodegenerative disorder and/or dementia, or a disease associated with glutamate-mediated excitotoxicity.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### EXAMPLES

### Example 1—Exemplary Pharmaceutical Compositions for the Treatment or Prevention of Dementia

Exemplary combinations according to the present disclosure are presented in Table 2.

### Example 2—Exemplary Methods for the Treatment or Prevention of Dementia

An exemplary method for the treatment or prevention of dementia according to the present disclosure is as follows:

Composition of Example 1: oral administration or co-administration of active agents (e.g., CBD and donepezil) to the subject nightly (i.e., between 10 to 60 minutes before sleep).

### Example 3—General Materials and Methods for Pre-Clinical Assessment of Pharmaceutical Compositions for the Treatment of Neurodegenerative Disorders and/or Dementia

### Cell Line

The SH-S5Y5 neuroblastoma cell line was used for pre-clinical analysis. The SH-S5Y5 cell line is commonly used as a model for neurodegenerative disorders as the cells can be converted to various types of functional neurons by the addition of specific compounds (see, e.g., Xicoy et al., 2017, Molecular Neurodegeneration, 12: 10). In addition, the SH-S5Y5 cell line has been used widely in experimental neurological studies, including analysis of neuronal differentiation, metabolism, and function related to neurodegenerative processes, neurotoxicity and neuroprotection (see, e.g., Datki et al., 2003, Brain Research Bulletin, 62(3): 22-229; and Xie et al., 2010, Chinese Medical Journal, 123(8): 1086-1092).

### MTT Assay

100 μL MTT reagent was added to each well (final concentration 0.5 mg/mL). Cells were then incubated at 37 °C, 5% CO₂ for 4 hours. Solubilization buffer (100 μL) was then added to each well and further incubated at 37 °C, 5% CO₂ for 24 hours. Absorbance was then measured by spectrophotometer at 550-600 nm and background corrected at 650 nm.

### Preparation of β-Amyloid

Amyloid-β (Aβ; 1 mg) was dissolved in 2 mL of 1,1,1,3,3,3-hexafluoro-2-propanol (HFIP). An additional 200 μL of HFIP was then added due to volatility. The Aβ was sealed with parafilm and vortexed for 30 seconds before aliquoting at 100 μL per 1.5 mL tube. HFIP was then evaporated in fume hood for 8 hours. Tubes were then sealed with lid and parafilm and stored at −80 °C. Prior to use, Aβ was reconstituted in DMSO to a final volume of 10% v/v for 10-15 min at RT and vortexed every 3-5 min. F-12 medium was then added to a final volume of 90% v/v. Aβ was then stored at 4 °C for 24 hours and vortexed before use.

### Preparation of Pharmaceutical Composition

Cannabidiol (CBD) was dissolved in dimethyl sulfoxide (DMSO) to a concentration of 10 mM before dilution to a working stock in water or medium, and used at final concentrations of 2.5, 5 and 10 μM in microplate assays. Donepezil was diluted in water to a concentration of 10 mM before dilution to a working stock, and used at final concentrations of 2.5, 5, 10, 20 and 30 μM in microplate assays.

### Viability Assays

Cells were seeded in 96-well microplates and incubated 37 °C, 5% CO₂ for 24 hours. Cells were then treated with the combination of CBD and donepezil at various concentrations, before further incubation at 37 °C, 5% CO₂ for 24 hours before viability was assessed by MTT assay.

For AR viability assays, A-β prepared as described above was added to each well prior to the addition of the combination of CBD and donepezil to a final concentration of 30 μM.

For glutamate toxicity assays, glutamate was added to each well prior to the addition of the combination of CBD and donepezil to a final concentration of 40 mM.

### Synergy Calculations

The predicted combinatorial effects of CBD and donepezil were calculated using the Bliss independence principle using the equation:

E_{pred}A+B=(E_{A}+E_{B})−(E_{A}E_{B})

The activity of the combination for this equation was calculated as:

(X-control)/(untreated−control)

Where untreated=media only cells not subjected to Aβ; and control=cells treated with Aβ only.

This calculation was performed for each treatment in each biological replicate, and the average of the biological replicate treatments was used to calculate the bliss independence principle. Two or three technical replicates were performed in each biological replicate.

RNA Extraction and cDNA Synthesis

Total RNA was isolated from cells using TRIzol in accordance with the manufacturer's instructions (Invitrogen, Carlsbad, CA, USA). RNA concentration and quality was assessed using a NanoDrop spectrophotometer. Only samples with a 260/280 ratio of ≥1.8 were used for further analysis.

Messenger RNA (mRNA) was purified from total RNA using poly-T oligo attached magnetic beads. After fragmentation, cDNA synthesis was performed in accordance with the manufacturer's instructions (Illumina, San Diego, CA, USA) to generate a directional and non-directional library. Libraries were checked with Qubit, real-time PCR was performed for quantification, and size distribution detection was performed using the Bioanalyzer. Quantified libraries were pooled and sequenced on an Illumina platform, according to the manufacturer's instructions.

### RNAseq

RNA sequencing was performed on samples obtained from SH-SY5Y cells treated with Aβ (20 μM), CBD, donepezil, or combinations of CBD and donepezil at various concentrations.

Raw reads were filtered and mapped to human reference genome (GRCh38/hg38) using HISAT2. Differential gene expression analysis was performed using R Package DEG seq2. Differentially expressed genes were identified by comparing mean fragments per kilobase of transcript per million fragments mapped (FPKM) from control cells (Aβ only) relative to CBD, donepezil or combination treated cells. Genes identified as significantly differentially expressed were further analysed for enrichment of genes known to be associated with dementia/neurodegenerative disorder or inflammation by gene ontology. Manual annotation of genes previously associated with specific neurodegenerative disorders and dementias was performed following a text mining analysis.

### Example 4—Pre-Clinical Assessment of Pharmaceutical Compositions for the Treatment of Alzheimer's Disease

The combinatorial neuroprotective effects of CBD and donepezil were assessed using an in vitro model of Alzheimer's disease. Briefly, SH-S5Y5 neuroblastoma cells were seeded into 96-well plates before being treated with a combination of CBD and donepezil in the presence of Aβ. Cell viability was assessed using MTT assay absorbance that was converted to cell viability (%), as shown in Tables 3 to 17.

Synergy between two agents occurs when the difference between the observed activity of the agents in combination is greater than the predicted activity. Based on these measures, the combination of CBD and donepezil synergize to improve cell viability in the presence of Aβ in vitro (Table 18).

### Example 5—Pre-Clinical Assessment of Pharmaceutical Compositions for the Treatment of Diseases and Disorders Associated with Glutamate Excitotoxicity

As described elsewhere herein, glutamate-mediated excitotoxicity is known to play a significant role in the pathogenesis of neurodegenerative disorders, including Parkinson's disease, Alzheimer's disease, Huntington's disease and amyotrophic lateral sclerosis (ALS). It is also recognised that glutamate-mediated excitotoxicity can cause neuronal cell death in response to severe insults, such as cerebral ischemia, traumatic brain injury (TBI), hypoglycaemia and status epilepticus.

To assess whether the combination of CBD and donepezil could mitigate or inhibit glutamate-mediated excitotoxicity, SH-S5Y5 neuroblastoma cells were seeded into 96-well plates before being treated with a combination of CBD and donepezil in the presence of glutamate. Cell viability was assessed using MTT assay absorbance, as shown in Table 19. These data demonstrate that the combination of CBD and donepezil significantly improves cell viability in the presence of glutamate in vitro.

### Example 6—Gene Expression Analysis of Neuronal Cells Treated with CBD and Donepezil

To further evaluate the mechanism by which CBD and donepezil synergize to mitigate or prevent neuronal cell death, RNA sequencing analysis was performed. Similar to the approach in Example 4, SH-S5Y5 neuroblastoma cells were treated with CBD, donepezil and combinations of CBD and donepezil in the presence of Aβ before RNA was extracted for analysis.

The combination of CBD and donepezil significantly modulated expression of key genes that have been previously identified as associated with one or more neurodegenerative disorders. When compared with the significantly modulated genes following treatment with CBD or donepezil alone, at least 21 genes were identified as uniquely modulated following treatment with the combination of CBD and donepezil, including APOE, ATN1, C19orf12, CFAP410, CTSD, DLG4, MT-TF, MT-TS1, NEKI, NIPA1, NOS2, NRXN1, TARDBP, CCDC88C, DNAH9, DTNBP1, ERCC8, HGSNAT, HTT, UBQLN4 and UCHL1 (Table 20).

Gene ontology analysis and text mining was performed for the significantly modulated genes to identify key pathways or regulators that are associated with neurodegenerative disorders and inflammation. Expression of genes APOE, DLG4 and NRXN1 are associated with postsynaptic membrane organisation, NMDA glutamate receptor clustering, AMPA glutamate receptor clustering and neurotransmitter-gated ion channel clustering. Moreover, HTT encodes for the Huntingtin protein, which is the gene linked to Huntington's disease. Furthermore, as shown in Table 20, the combination of CBD and donepezil significantly modulated genes that are associated with a range of different neurodegenerative disorders and dementias, including Parkinson's disease, Alzheimer's disease, dementia, vascular dementia, ALS, TBI with dementia, frontotemporal dementia, neurodegeneration with brain iron accumulation, mitochondrial dysfunction, dementia with Lewy bodies, Huntington's disease, ataxia, and neuronal ceroid lipofuscinosis (NCL).

### CONCLUSION

The data presented herein demonstrate that the combination of CBD and donepezil synergize to provide a protective effect to neuronal cells that are exposed to Aβ, and in some instances, can partially rescue cells from the toxic effects of Aβ (see, e.g., 10 M CBD+10 M donepezil; 10 M CBD+20 M donepezil; and 10 M CBD+30 M donepezil). Moreover, the combination of CBD and donepezil has also been shown to provide a protective effect against neuronal cell death following exposure to glutamate.

Gene expression analysis has demonstrated that the combination of CBD and donepezil modulates genes associated with a range of different neurodegenerative disorders and dementias, including Parkinson's disease, Alzheimer's disease, dementia, vascular dementia, ALS, TBI with dementia, frontotemporal dementia, neurodegeneration with brain iron accumulation, mitochondrial dysfunction, dementia with Lewy bodies, Huntington's disease, ataxia, and NCL.

Taken together, these data are enabling for methods for the treatment or prevention of at least one neurodegenerative disorder, dementia (e.g., Parkinson's disease, Alzheimer's disease, dementia, vascular dementia, ALS, TBI with dementia, frontotemporal dementia, neurodegeneration with brain iron accumulation, mitochondrial dysfunction, dementia with Lewy bodies, Huntington's disease, ataxia, and NCL), and diseases associated with glutamate-mediated excitotoxicity (e.g., acute diseases such as ischemia, TBI, epilepsy, hypoglycaemia, and status epilepticus; and chronic neurodegenerative disorders such as Parkinson's disease, Alzheimer's disease, Huntington's disease and ALS), and compositions comprising CBD and an acetylcholinesterase inhibitor (e.g., donepezil).

TABLE 1
Exemplary structures of CBD or pharmaceutically acceptable salts or
derivatives thereof

| Molecule | Structure |
|---|---|
| Cannabidiol (CBD); Formula (I) | 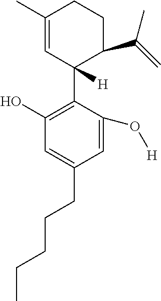 [SMILES: C=C(C)[C@@H]1CCC(C)=C[C@H]1c1c(O)cc(CCCCC)cc1O] |
| Cannabidiolic acid (CBDA); Formula (II) | 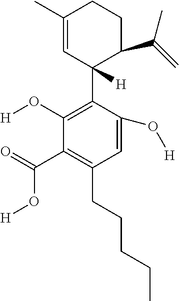 [SMILES: C=C(C)[C@@H]1CCC(C)=C[C@H]1c1c(O)cc(CCCCC)c(C(=O)O)c1O] |
| 7-Hydroxycannabidiol; Formula (III) | 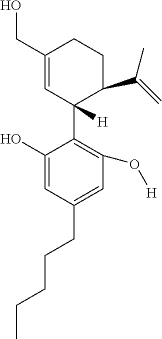 [SMILES: C=C(C)[C@@H]1CCC(CO)=C[C@H]1c1c(O)cc(CCCCC)cc1O] |
| Cannabidiol dimethyl ether (CBDD); Formula (IV) | 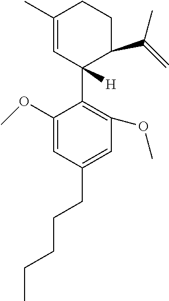 [SMILES: C=C(C)[C@@H]1CCC(C)=C[C@H]1c1c(OC)cc(CCCCC)cc1OC] |
| Cannabidiorcol (CBD-C₁); Formula (V) | 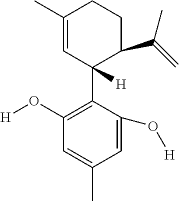 [SMILES: C=C(C)[C@@H]1CCC(C)=C[C@H]1c1c(O)cc(C)cc1O] |

TABLE 2
Exemplary pharmaceutical compositions for
the treatment or prevention of dementia

| Pharmaceutical | | | |
|---|---|---|---|
| composition | Active agent | Dose range | |
| A | CBD | 20-50 | mg |
| | Donepezil HCl | 10-25 | mg |
| B | CBD | 50-500 | mg |
| | Donepezil HCl | 10-25 | mg |
| C | CBD | 600-1500 | mg |
| | Donepezil HCl | 10-25 | mg |
| D | CBD | 20-50 | mg |
| | Donepezil HCl | 5-10 | mg |
| E | CBD | 50-500 | mg |
| | Donepezil HCl | 5-10 | mg |
| F | CBD | 500-1500 | mg |
| | Donepezil HCl | 5-10 | mg |

TABLE 3
Cell viability in response to Aβ following treatment with
a combination of 2.5 μM CBD and 2.5 μM donepezil

| | | E (relative viability |
|---|---|---|
| | Cell viability as | value used to |
| Treatment | measured by MTT | calculate synergy) |
| Untreated (no Aβ, | 1 | 1 |
| media only) | | |
| Control (20 μM Aβ) | 0.653 | 0 |
| CBD 2.5 μM | 0.011 | 0.049 |
| Donepezil 2.5 μM | −0.036 | −0.129 |
| CBD 2.5 μM + | 0.035 | 0.089 |
| donepezil 2.5 μM | | |

TABLE 4
Cell viability in response to Aβ following treatment with
a combination of 2.5 μM CBD and 5.0 μM donepezil

| | | E (relative viability |
|---|---|---|
| | Cell viability as | value used to |
| Treatment | measured by MTT | calculate synergy) |
| Untreated (no Aβ, | 1 | 1 |
| media only) | | |
| Control (20 μM Aβ) | 0.653 | 0 |
| CBD 2.5 μM | 0.011 | 0.049 |
| Donepezil 5.0 μM | −0.021 | −0.072 |
| CBD 2.5 μM + | 0.049 | 0.156 |
| donepezil 5.0 μM | | |

TABLE 5
Cell viability in response to Aβ following treatment with
a combination of 2.5 μM CBD and 10 μM donepezil

| | | E (relative viability |
|---|---|---|
| | Cell viability as | value used to |
| Treatment | measured by MTT | calculate synergy) |
| Untreated (no Aβ, | 1 | 1 |
| media only) | | |
| Control (20 μM Aβ) | 0.653 | 0 |
| CBD 2.5 μM | 0.011 | 0.049 |
| Donepezil 10 μM | 0.021 | −0.013 |
| CBD 2.5 μM + | 0.043 | 0.067 |
| donepezil 10 μM | | |

TABLE 6
Cell viability in response to Aβ following treatment with
a combination of 2.5 μM CBD and 20 μM donepezil

| | | E (relative viability |
|---|---|---|
| | Cell viability as | value used to |
| Treatment | measured by MTT | calculate synergy) |
| Untreated (no Aβ, | 1 | 1 |
| media only) | | |
| Control (20 μM Aβ) | 0.653 | 0 |
| CBD 2.5 μM | 0.011 | 0.049 |
| Donepezil 20 μM | −0.011 | −0.073 |
| CBD 2.5 μM + | 0.013 | 0.041 |
| donepezil 20 μM | | |

TABLE 7
Cell viability in response to Aβ following treatment with
a combination of 2.5 μM CBD and 30 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 2.5 μM | 0.011 | 0.049 |
| Donepezil 30 μM | −0.013 | −0.065 |
| CBD 2.5 μM + | 0.039 | 0.064 |
| donepezil 30 μM | | |

TABLE 8
Cell viability in response to Aβ following treatment with
a combination of 5.0 μM CBD and 2.5 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 5 μM | 0.031 | 0.092 |
| Donepezil 2.5 μM | −0.036 | −0.129 |
| CBD 5 μM + | 0.044 | 0.163 |
| donepezil 2.5 μM | | |

TABLE 9
Cell viability in response to Aβ following treatment with
a combination of 5.0 μM CBD and 5.0 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 5 μM | 0.031 | 0.092 |
| Donepezil 5 μM | −0.021 | −0.072 |
| CBD 5 μM + | 0.064 | 0.186 |
| donepezil 5 μM | | |

TABLE 10
Cell viability in response to Aβ following treatment with
a combination of 5.0 μM CBD and 10 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 5 μM | 0.031 | 0.092 |
| Donepezil 10 μM | 0.021 | −0.013 |
| CBD 5 μM + | 0.070 | 0.227 |
| donepezil 10 μM | | |

TABLE 11
Cell viability in response to Aβ following treatment with
a combination of 5.0 μM CBD and 20 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 5 μM | 0.031 | 0.092 |
| Donepezil 20 μM | −0.011 | −0.073 |
| CBD 5 μM + | 0.056 | 0.172 |
| donepezil 20 μM | | |

TABLE 12
Cell viability in response to Aβ following treatment with
a combination of 5.0 μM CBD and 30 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 5 μM | 0.031 | 0.092 |
| Donepezil 30 μM | −0.013 | −0.065 |
| CBD 5 μM + | 0.100 | 0.232 |
| donepezil 30 μM | | |

TABLE 13
Cell viability in response to Aβ following treatment with
a combination of 10 μM CBD and 2.5 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 10 μM | 0.116 | 0.458 |
| Donepezil 2.5 μM | −0.036 | −0.129 |
| CBD 10 μM + | 0.154 | 0.589 |
| donepezil 2.5 μM | | |

TABLE 14
Cell viability in response to Aβ following treatment with
a combination of 10 μM CBD and 5.0 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 10 μM | 0.116 | 0.458 |
| Donepezil 5 μM | −0.021 | −0.072 |
| CBD 10 μM + | 0.174 | 0.654 |
| donepezil 5 μM | | |

TABLE 15
Cell viability in response to Aβ following treatment with
a combination of 10 μM CBD and 10 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 10 μM | 0.116 | 0.458 |
| Donepezil 10 μM | 0.021 | −0.013 |
| CBD 10 μM + | 0.201 | 0.778 |
| donepezil 10 μM | | |

TABLE 16
Cell viability in response to Aβ following treatment with
a combination of 10 μM CBD and 20 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 10 μM | 0.116 | 0.458 |
| Donepezil 20 μM | −0.011 | −0.073 |
| CBD 10 μM + | 0.181 | 0.721 |
| donepezil 20 μM | | |

TABLE 17
Cell viability in response to Aβ following treatment with
a combination of 10 μM CBD and 30 μM donepezil

| | Cell viability as | E (relative viability value |
|---|---|---|
| Treatment | measured by MTT | used to calculate synergy) |
| Untreated (no Aβ, | 1 | 1 |
| media only) | | |
| Control (20 μM Aβ) | 0.653 | 0 |
| CBD 10 μM | 0.116 | 0.458 |
| Donepezil 30 μM | −0.013 | −0.065 |
| CBD 10 μM + | 0.184 | 0.694 |
| donepezil 30 μM | | |

TABLE 18
Synergistic combinations of CBD and donepezil

| CBD | | Donepezil (μM) | | | | |
|---|---|---|---|---|---|---|
| (μM) | | 2.5 | 5 | 10 | 20 | 30 |
| 2.5 | Observed | 0.089 | 0.156 | 0.067 | 0.041 | 0.064 |
| | combination | | | | | |
| | effect | | | | | |
| | Predicted | −0.073 | −0.019 | 0.037 | −0.020 | −0.012 |
| | combination | | | | | |
| | effect | | | | | |
| | Observed − | 0.162* | 0.175* | 0.029 | 0.061* | 0.077* |
| | predicted effect | | | | | |
| 5 | Observed | 0.163 | 0.186 | 0.227 | 0.172 | 0.232 |
| | combination | | | | | |
| | effect | | | | | |
| | Predicted | −0.026 | 0.026 | 0.080 | 0.025 | 0.033 |
| | combination | | | | | |
| | effect | | | | | |
| | Observed − | 0.189* | 0.160* | 0.147* | 0.147* | 0.199* |
| | predicted effect | | | | | |
| 10 | Observed | 0.589 | 0.654 | 0.778 | 0.721 | 0.694 |
| | combination | | | | | |
| | effect | | | | | |
| | Predicted | 0.388 | 0.418 | 0.451 | 0.418 | 0.422 |
| | combination | | | | | |
| | effect | | | | | |
| | Observed − | 0.202 | 0.236 | 0.327 | 0.304 | 0.272 |
| | predicted effect | | | | | |

*Indicates synergistic combinations.

TABLE 19
Cell viability in response to glutamate following
treatment with combinations of CBD and donepezil

| | Mean absorbance as | | P- |
|---|---|---|---|
| Treatment | measured by MTT | SD | value\hat{ } |
| Untreated (media | 0.512 | 0.080 | |
| only) | | | |
| Control (40 mM | 0.184 | 0.012 | |
| L-glutamate) | | | |
| CBD 10 μM + | 0.349 | 0.025 | 0.022* |
| donepezil 10 μM | | | |
| CBD 10 μM + | 0.279 | 0.071 | 0.295 |
| donepezil 20 μM | | | |
| CBD 10 μM + | 0.332 | 0.019 | 0.017* |
| donepezil 30 μM | | | |

\hat{ }P-values calculated from two-tailed t-test between control and treatment, two-sample unequal variance;
*p < 0.05.

TABLE 20
Genes significantly modulated following treatment with a combination
of 5 μM CBD and 5 μM donepezil in the presence of Aβ

| | | | | Associated |
|---|---|---|---|---|
| | | CBD + | P- | neurodegenerative |
| Gene | Control | DON | value | disorder(s) |
| ACE | 0.208785 | 0.590006 | 0.024184 | PD, AD, D, VD |
| ACHE | 20.557 | 14.128 | 0.009 | AD, ALS, D, |
| | | | | VD, TBI-D |
| APBB2 | 7.376 | 5.091 | 0.015 | AD |
| APOE* | 3.425 | 2.007 | 0.043 | AD, ALS, D |
| APP | 293.865 | 151.659 | 0.027 | AD, ALS, D, |
| | | | | FTD, VD |
| ATN1* | 89.813 | 70.299 | 0.023 | |
| ATXN1 | 0.851 | 1.160 | 0.032 | AD, ALS, AX |
| BDNF | 2.554 | 0.337 | 0.017 | NDID |
| C19orf12* | 9.324 | 6.688 | 0.034 | AD, D, VD |
| C9orf72 | 3.921 | 8.354 | 0.003 | AD, ALS |
| CDK1 | 18.213 | 29.732 | 0.006 | AD |
| CFAP410* | 2.736 | 2.046 | 0.015 | AD, D |
| CHRNA3 | 26.776 | 43.692 | 0.018 | AD, ALS, D, |
| | | | | FTD, TBI-D |
| CHRNB4 | 1.624 | 4.438 | 0.012 | |
| CLN8 | 3.580 | 2.750 | 0.007 | NDID |
| COASY | 38.304 | 25.320 | 0.005 | D, VD |
| COL4A1 | 3.205 | 5.921 | 0.027 | |
| COMT | 8.395 | 7.548 | 0.003 | NDID |
| CSTB | 7.261 | 9.058 | 0.014 | AD |
| CTSD* | 21.381 | 16.880 | 0.046 | |
| CYP27A1 | 2.685 | 0.628 | 0.033 | NDID |
| DLG4* | 17.764 | 12.022 | 0.025 | |
| DMD | 0.166 | 0.370 | 0.026 | |
| DNAH8 | 0.090 | 0.014 | 0.020 | |
| DNAJB6 | 19.482 | 14.472 | 0.026 | D, VD |
| DNAJC6 | 11.205 | 13.813 | 0.048 | AD, MTD |
| DRD2 | 2.532 | 5.019 | 0.019 | |
| GBA2 | 53.427 | 40.336 | 0.007 | AD, ALS |
| GM2A | 2.985 | 5.751 | 0.005 | AD |
| GRN | 43.913 | 36.989 | 0.008 | ALS |
| GSR | 10.993 | 13.367 | 0.030 | D, FTD |
| HNRNPA1 | 180.363 | 263.488 | 0.008 | AD, ALS |
| HNRNPDL | 44.506 | 51.158 | 0.029 | AD |
| HTRA1 | 8.986 | 2.662 | 0.022 | AD, D, VD |
| IFT74 | 3.453 | 4.480 | 0.034 | |
| KCTD7 | 18.066 | 12.520 | 0.003 | |
| LRP10 | 23.073 | 16.624 | 0.004 | AD |
| MADD | 24.804 | 17.412 | 0.019 | |
| MAP1B | 25.187 | 34.312 | 0.005 | |
| MAP2 | 9.232 | 14.308 | 0.006 | AD |
| MAPK8 | 7.482 | 10.247 | 0.004 | AD |
| MECP2 | 6.880 | 5.081 | 0.015 | AD |
| MT-ND6 | 281.366 | 101.142 | 0.004 | |
| MTR | 7.739 | 10.194 | 0.017 | |
| MT-TA | 6.758 | 2.582 | 0.019 | |
| MT-TF* | 6.207 | 2.811 | 0.032 | |
| MT-TN | 19.578 | 8.368 | 0.008 | MTD |
| MT-TS1* | 112.2448 | 62.5049 | 0.022294 | AD, ALS, TBI-D |
| NEFL | 13.063 | 24.557 | 0.003 | ALS |
| NEK1* | 3.046 | 3.955 | 0.005 | AD |
| NGF | 14.274 | 1.249 | 0.013 | |
| NIPA1* | 12.077 | 9.017 | 0.042 | AD, ALS |
| NOS2* | 0.294 | 0.494 | 0.038 | D, VD |
| NRXN1* | 0.084 | 0.171 | 0.017 | AD, ALS |
| NTRK2 | 0.329 | 1.037 | 0.010 | AD |
| OPA1 | 5.517 | 9.928 | 0.001 | AD, MTD |
| PANK2 | 12.767 | 6.260 | 0.019 | AD |
| PARK7 | 54.907 | 62.896 | 0.039 | ALS, D, FTD |
| PDGFRB | 32.930 | 20.124 | 0.001 | |
| PDLIM3 | 1.777 | 3.310 | 0.023 | |
| PHF1 | 31.064 | 18.841 | 0.011 | AD |
| PNPLA6 | 25.949 | 14.820 | 0.008 | ALS |
| PSEN1 | 12.549 | 10.601 | 0.044 | AD, ALS, D, |
| | | | | FTD |
| RETREG1 | 0.386 | 0.738 | 0.008 | |
| RRM2B | 28.427 | 14.155 | 0.021 | MTD |
| SETD1A | 22.012 | 11.773 | 0.022 | |
| SMCR8 | 16.386 | 9.070 | 0.017 | |
| SOD1 | 44.41255 | 53.88653 | 0.039861 | AD, ALS, D |
| SORT1 | 11.268 | 9.886 | 0.047 | AD |
| SYP | 20.006 | 15.359 | 0.024 | AD |
| TAF15 | 14.968 | 18.873 | 0.027 | ALS |
| TARDBP* | 16.647 | 19.966 | 0.000 | AD, ALS, D, |
| | | | | FTD |
| TFEB | 4.181 | 2.806 | 0.004 | |
| TRIM9 | 0.529 | 0.998 | 0.018 | |
| TWNK | 25.053 | 14.653 | 0.010 | |
| U2AF2 | 88.738 | 50.869 | 0.011 | |
| WDR45 | 18.358 | 11.000 | 0.012 | NDID |
| XPR1 | 31.247 | 53.072 | 0.013 | |
| A2M | 0.242 | 0.146 | 0.004 | AD |
| ATP6V1A | 18.408 | 18.117 | 0.037 | |
| ATP6V1E1 | 51.257 | 43.971 | 0.025 | |
| CCDC88C* | 1.912 | 2.246 | 0.023 | |
| CDKL5 | 1.735 | 2.027 | 0.004 | |
| CLN5 | 0.695 | 0.694 | 0.004 | PD, NCL |
| COL3A1 | 34.958 | 36.355 | 0.003 | |
| DDHD2 | 6.006 | 6.053 | 0.025 | |
| DIAPH1 | 23.288 | 24.693 | 0.021 | |
| DLG2 | 1.977 | 2.288 | 0.013 | |
| DNAH9* | 0.024 | 0.032 | 0.004 | |
| DNAJC5 | 16.143 | 18.404 | 0.040 | D |
| DPP10 | 1.590 | 1.474 | 0.044 | |
| DTNBP1* | 4.826 | 4.170 | 0.038 | |
| EIF2B4 | 17.110 | 16.745 | 0.023 | |
| EIF4G1 | 46.194 | 51.809 | 0.013 | |
| ERCC8* | 2.075 | 2.229 | 0.035 | |
| FIG4 | 6.430 | 5.164 | 0.001 | ALS, FTD |
| GPT2 | 9.062 | 9.168 | 0.018 | |
| HGSNAT* | 3.455 | 4.180 | 0.036 | |
| HTT* | 5.419 | 6.119 | 0.045 | ALS, FTD, AD, |
| | | | | PD, D, LBD, HD, |
| | | | | NDIA |
| KATNAL2 | 2.005 | 1.765 | 0.038 | |
| LMNB1 | 10.575 | 11.219 | 0.007 | |
| LRRC56 | 0.982 | 0.996 | 0.008 | |
| MTR | 8.443 | 8.810 | 0.017 | AD, PD |
| MT-TH | 1.782 | 0.559 | 0.047 | MTD |
| MT-TM | 12.408 | 13.655 | 0.018 | MD |
| MT-TQ | 37.146 | 31.997 | 0.021 | MTD |
| MT-TV | 17.283 | 23.246 | 0.001 | |
| NEAT1 | 10.972 | 10.736 | 0.040 | |
| NID1 | 38.202 | 40.383 | 0.050 | |
| NTNG1 | 0.327 | 0.336 | 0.001 | |
| OTUD5 | 29.629 | 30.134 | 0.016 | |
| PDE10A | 3.855 | 3.397 | 0.040 | HD |
| PDGFRA | 11.237 | 11.526 | 0.009 | |
| PLA2G6 | 0.634 | 0.656 | 0.019 | |
| PMPCA | 13.790 | 12.763 | 0.003 | |
| PODXL | 4.194 | 4.389 | 0.006 | |
| PRICKLE1 | 3.757 | 3.750 | 0.002 | |
| PRICKLE2 | 0.060 | 0.055 | 0.011 | |
| PRKAR1B | 13.838 | 15.117 | 0.012 | |
| RAB33A | 6.793 | 5.587 | 0.023 | |
| RELN | 9.086 | 9.079 | 0.017 | |
| RNF216 | 10.517 | 11.641 | 0.030 | |
| SERPINI1 | 12.857 | 11.253 | 0.022 | AD, FTD, D, HD |
| SLC2A1 | 8.472 | 9.369 | 0.024 | |
| SMARCA2 | 2.546 | 2.571 | 0.021 | |
| SMARCC1 | 21.060 | 23.609 | 0.011 | AD |
| SORCS1 | 24.655 | 26.220 | 0.015 | |
| SORL1 | 0.455 | 0.477 | 0.019 | |
| TCF4 | 32.383 | 33.412 | 0.037 | |
| TTC21B | 4.351 | 4.516 | 0.030 | |
| TUBA4A | 0.334 | 0.266 | 0.012 | FTD, ALS |
| UBQLN4* | 38.930 | 42.857 | 0.028 | |
| UCHL1* | 229.489 | 218.586 | 0.043 | |

Genes marked with * are uniquely modulated following treatment with the combination of CBD and donepezil (i.e., relative to single agent treatment with CBD or donepezil, Tables 21 and 22).
AD—Alzheimer's disease; PD—Parkinson's disease; D—dementia; VD—vascular dementia; ALS—Amyotrophic lateral sclerosis; TBI-D—traumatic brain injury with dementia; FTD—frontotemporal dementia; NDIA—neurodegeneration with brain iron accumulation; MTD—mitochondrial dysfunction; LBD—dementia with Lewy bodies; HD—Huntington's disease; AX—ataxia; NCL—neuronal ceroid lipofuscinosis.

TABLE 21
Genes significantly modulated following treatment
with 5 μM CBD in the presence of Aβ

| Gene | Control | CBD | P-value |
|---|---|---|---|
| LAMP1 | 34.350 | 31.452 | 0.018 |
| MBP | 0.089 | 0.046 | 0.012 |
| TAF15 | 14.968 | 17.887 | 0.046 |
| A2M | 0.242 | 0.173 | 0.024 |
| ERCC4 | 1.970 | 2.197 | 0.037 |
| GBE1 | 1.194 | 1.305 | 0.037 |
| IRF6 | 7.365 | 7.774 | 0.012 |
| MFSD8 | 2.553 | 2.603 | 0.009 |
| MT-TW | 2.846 | 4.309 | 0.023 |
| SLC20A2 | 7.014 | 7.447 | 0.046 |
| TAF15 | 15.869 | 17.095 | 0.046 |
| TRAPPC2 | 3.872 | 4.267 | 0.013 |

TABLE 22
Genes significantly modulated following treatment
with 5 μM donepezil in the presence of Aβ

| Gene | Control | DON | P-value |
|---|---|---|---|
| ABCA7 | 2.607 | 2.087 | 0.032 |
| ACE | 0.209 | 0.644 | 0.004 |
| ACHE | 20.557 | 15.012 | 0.010 |
| APBB2 | 7.376 | 5.038 | 0.015 |
| APP | 293.865 | 159.632 | 0.033 |
| ATP13A2 | 7.789 | 5.931 | 0.022 |
| ATXN1 | 0.851 | 1.152 | 0.002 |
| ATXN7 | 0.042 | 0.150 | 0.014 |
| BACE1 | 20.034 | 16.945 | 0.031 |
| BDNF | 2.554 | 0.255 | 0.014 |
| C9orf72 | 3.921 | 8.663 | 0.001 |
| CDK1 | 18.213 | 31.665 | 0.001 |
| CHRNA3 | 26.776 | 35.011 | 0.015 |
| CHRNB4 | 1.624 | 2.920 | 0.018 |
| CLN8 | 3.580 | 2.847 | 0.014 |
| COASY | 38.304 | 24.487 | 0.003 |
| COL4A1 | 3.205 | 6.341 | 0.014 |
| COMT | 8.395 | 7.242 | 0.009 |
| CREB1 | 6.333 | 7.340 | 0.006 |
| CSTB | 7.261 | 9.549 | 0.008 |
| CYP27A1 | 2.685 | 0.693 | 0.037 |
| DMD | 0.166 | 0.352 | 0.017 |
| DNAH8 | 0.090 | 0.009 | 0.017 |
| DNAJB6 | 19.482 | 13.438 | 0.010 |
| DNAJC6 | 11.205 | 14.537 | 0.017 |
| DRD2 | 2.532 | 4.790 | 0.031 |
| EIF2B5 | 14.483 | 12.247 | 0.017 |
| GAP43 | 8.840 | 14.884 | 0.016 |
| GBA2 | 53.427 | 42.069 | 0.010 |
| GM2A | 2.985 | 5.621 | 0.005 |
| GRN | 43.913 | 38.715 | 0.009 |
| GSR | 10.993 | 14.077 | 0.009 |
| HNRNPA1 | 180.363 | 274.269 | 0.003 |
| HNRNPDL | 44.506 | 58.385 | 0.001 |
| HTRA1 | 8.986 | 3.452 | 0.035 |
| IFT74 | 3.453 | 4.207 | 0.032 |
| KCTD7 | 18.066 | 14.737 | 0.016 |
| LRP10 | 23.073 | 19.237 | 0.013 |
| MADD | 24.804 | 17.028 | 0.015 |
| MAP1B | 25.187 | 31.197 | 0.004 |
| MAP2 | 9.232 | 13.284 | 0.018 |
| MAPK8 | 7.482 | 9.392 | 0.022 |
| MECP2 | 6.880 | 5.530 | 0.033 |
| MME | 0.091 | 0.240 | 0.004 |
| MT-ND6 | 281.366 | 64.249 | 0.001 |
| MT-TA | 6.758 | 2.804 | 0.027 |
| MT-TN | 19.578 | 4.546 | 0.001 |
| MTR | 7.739 | 9.723 | 0.018 |
| NDUFS4 | 23.590 | 31.457 | 0.040 |
| NEFL | 13.063 | 22.943 | 0.010 |
| NGF | 14.274 | 1.165 | 0.012 |
| NTRK2 | 0.329 | 0.700 | 0.015 |
| OPA1 | 5.517 | 9.622 | 0.001 |
| PANK2 | 12.767 | 5.414 | 0.010 |
| PARK7 | 54.907 | 62.824 | 0.006 |
| PDGFRB | 32.930 | 20.075 | 0.001 |
| PDLIM3 | 1.777 | 3.231 | 0.026 |
| PHF1 | 31.064 | 22.592 | 0.022 |
| PINK1 | 12.825 | 9.640 | 0.001 |
| PLAU | 0.799 | 1.790 | 0.005 |
| PNPLA6 | 25.949 | 14.472 | 0.007 |
| PRDM8 | 4.779 | 3.461 | 0.013 |
| PSEN1 | 12.549 | 10.463 | 0.033 |
| RETREG1 | 0.386 | 0.774 | 0.001 |
| RRM2B | 28.427 | 15.106 | 0.027 |
| SCARB2 | 16.783 | 20.762 | 0.011 |
| SETD1A | 22.012 | 11.489 | 0.020 |
| SMCR8 | 16.386 | 9.923 | 0.025 |
| SOD1 | 44.413 | 54.134 | 0.023 |
| SORT1 | 11.268 | 9.054 | 0.004 |
| SYP | 20.006 | 15.060 | 0.004 |
| TFEB | 4.181 | 2.618 | 0.001 |
| TRIM9 | 0.529 | 1.021 | 0.008 |
| TWNK | 25.053 | 15.384 | 0.010 |
| U2AF2 | 88.738 | 53.895 | 0.014 |
| ULK1 | 9.616 | 7.435 | 0.008 |
| WDR45 | 18.358 | 10.730 | 0.008 |
| XPR1 | 31.247 | 53.075 | 0.010 |
| A2M | 0.242 | 0.122 | 0.006 |
| ADAM10 | 6.563 | 5.824 | 0.019 |
| ADCY5 | 1.228 | 1.662 | 0.036 |
| AIFM1 | 11.948 | 10.769 | 0.026 |
| ALDH18A1 | 16.206 | 18.398 | 0.012 |
| ALMS1 | 2.170 | 2.220 | 0.003 |
| ANXA11 | 5.661 | 6.974 | 0.025 |
| APBB2 | 7.118 | 6.231 | 0.015 |
| APOL2 | 3.111 | 3.390 | 0.054 |
| APTX | 6.525 | 6.767 | 0.011 |
| ASTN2 | 8.739 | 10.467 | 0.008 |
| ATL1 | 2.891 | 3.208 | 0.027 |
| ATL3 | 7.656 | 6.616 | 0.030 |
| ATP1A3 | 2.847 | 3.739 | 0.021 |
| ATP6V0A2 | 4.281 | 4.607 | 0.017 |
| ATP6V1A | 18.408 | 16.105 | 0.038 |
| ATP6V1E1 | 51.257 | 41.488 | 0.048 |
| ATXN7L3B | 19.279 | 20.868 | 0.006 |
| AUH | 9.982 | 9.689 | 0.001 |
| BIRC6 | 16.315 | 14.545 | 0.045 |
| BLMH | 6.516 | 6.896 | 0.013 |
| C19orf12 | 8.149 | 8.062 | 0.002 |
| CARD14 | 0.139 | 0.106 | 0.051 |
| CDKL5 | 1.735 | 2.077 | 0.001 |
| CFAP43 | 0.931 | 0.636 | 0.049 |
| CHCHD10 | 7.586 | 11.163 | 0.010 |
| CLCN6 | 8.460 | 9.489 | 0.007 |
| CLN3 | 0.092 | 0.093 | 0.051 |
| CLN5 | 0.695 | 0.652 | 0.004 |
| COL3A1 | 34.958 | 29.884 | 0.008 |
| COL4A1 | 3.206 | 4.649 | 0.014 |
| COQ2 | 1.970 | 1.406 | 0.055 |
| CPT1C | 21.622 | 23.209 | 0.000 |
| CRAT | 7.227 | 9.692 | 0.031 |
| CST3 | 12.563 | 14.607 | 0.049 |
| CTSA | 11.863 | 12.014 | 0.001 |
| CTSF | 10.619 | 12.207 | 0.034 |
| CYLD | 4.607 | 3.887 | 0.036 |
| CYP51A1 | 1.068 | 1.389 | 0.020 |
| DCX | 3.114 | 3.488 | 0.003 |
| DDHD2 | 6.006 | 6.144 | 0.000 |
| DEGS1 | 32.375 | 28.878 | 0.003 |
| DEPDC5 | 0.656 | 0.683 | 0.020 |
| DIAPH1 | 23.288 | 23.289 | 0.016 |
| DISC1 | 1.302 | 1.242 | 0.033 |
| DLAT | 11.385 | 10.390 | 0.035 |
| DLG2 | 1.977 | 2.816 | 0.041 |
| DNAJC5 | 16.143 | 18.101 | 0.004 |
| DNAJC6 | 12.142 | 12.821 | 0.017 |
| DNMT1 | 19.641 | 19.982 | 0.019 |
| DPH1 | 3.762 | 3.962 | 0.040 |
| DPP10 | 1.590 | 1.562 | 0.009 |
| DPY19L2P2 | 0.469 | 0.560 | 0.040 |
| EEF1A2 | 63.840 | 77.336 | 0.016 |
| EHMT1 | 4.566 | 4.445 | 0.010 |
| EIF2B3 | 3.828 | 3.200 | 0.043 |
| EIF2B4 | 17.110 | 13.862 | 0.014 |
| EIF4G1 | 46.194 | 47.437 | 0.005 |
| FAR1 | 9.502 | 9.330 | 0.001 |
| FGFR2 | 0.353 | 0.228 | 0.053 |
| FHIT | 0.882 | 1.086 | 0.033 |
| FIG4 | 6.430 | 6.116 | 0.051 |
| FSD1L | 2.639 | 2.451 | 0.002 |
| FTL | 423.318 | 514.018 | 0.015 |
| FUS | 26.563 | 23.115 | 0.006 |
| FZD3 | 1.990 | 2.276 | 0.010 |
| GAN | 1.529 | 1.495 | 0.018 |
| GNAS | 193.754 | 202.087 | 0.003 |
| GOSR2 | 5.846 | 5.640 | 0.000 |
| GPT2 | 9.062 | 9.323 | 0.018 |
| HCFC1 | 17.149 | 21.365 | 0.014 |
| HEXB | 11.029 | 11.767 | 0.010 |
| HOTAIR | 2.219 | 1.982 | 0.008 |
| HRAS | 19.713 | 18.552 | 0.000 |
| HTRA2 | 26.112 | 24.427 | 0.003 |
| IRAK1BP1 | 0.866 | 0.848 | 0.030 |
| IRF4 | 0.076 | 0.010 | 0.050 |
| IRF6 | 7.365 | 5.933 | 0.000 |
| JAM2 | 5.256 | 4.131 | 0.040 |
| KATNAL2 | 2.005 | 2.056 | 0.000 |
| KCNT1 | 1.355 | 1.707 | 0.002 |
| KIF4A | 12.106 | 12.317 | 0.000 |
| KIF5A | 8.491 | 7.978 | 0.023 |
| LDLR | 5.953 | 7.563 | 0.003 |
| LIG3 | 4.935 | 5.232 | 0.007 |
| LMBRD1 | 5.367 | 5.309 | 0.022 |
| LMNB1 | 10.575 | 13.789 | 0.020 |
| LRRC56 | 0.982 | 1.129 | 0.002 |
| LYST | 3.000 | 2.468 | 0.053 |
| MAPT | 1.915 | 2.615 | 0.007 |
| MATR3 | 0.559 | 0.511 | 0.001 |
| MEF2C | 1.668 | 1.624 | 0.044 |
| MEG3 | 0.007 | 0.008 | 0.038 |
| MFSD8 | 2.553 | 2.381 | 0.038 |
| MIAT | 5.041 | 6.814 | 0.014 |
| MIR103A2 | 15.688 | 9.508 | 0.029 |
| MMACHC | 1.874 | 1.731 | 0.018 |
| MMADHC | 29.814 | 28.376 | 0.009 |
| MRE11 | 4.546 | 4.436 | 0.036 |
| MSRA | 1.969 | 1.709 | 0.027 |
| MT-ATP6 | 6754.328 | 5525.655 | 0.009 |
| MT-ATP8 | 388.214 | 298.175 | 0.017 |
| MT-CO3 | 6360.482 | 6139.890 | 0.003 |
| MT-CYB | 4523.043 | 4140.601 | 0.003 |
| MT-ND1 | 2654.440 | 2177.390 | 0.010 |
| MT-ND2 | 2855.888 | 2011.949 | 0.019 |
| MT-ND5 | 1304.948 | 1379.984 | 0.012 |
| MTR | 8.443 | 8.954 | 0.018 |
| MT-TC | 6.838 | 10.995 | 0.052 |
| MT-TD | 1.849 | 2.334 | 0.032 |
| MT-TE | 8.733 | 14.784 | 0.049 |
| MT-TH | 1.782 | 0.388 | 0.047 |
| MT-TI | 5.685 | 4.404 | 0.038 |
| MT-TM | 12.408 | 7.334 | 0.006 |
| MT-TP | 74.063 | 57.524 | 0.023 |
| MT-TQ | 37.146 | 28.576 | 0.001 |
| MT-TR | 9.625 | 7.395 | 0.053 |
| MT-TS1 | 108.757 | 75.947 | 0.004 |
| MT-TV | 17.283 | 9.882 | 0.000 |
| MT-TW | 2.846 | 2.620 | 0.019 |
| MT-TY | 19.436 | 14.109 | 0.034 |
| NBEA | 11.016 | 12.180 | 0.010 |
| NEAT1 | 10.972 | 8.854 | 0.010 |
| NEFH | 14.777 | 13.335 | 0.018 |
| NID1 | 38.202 | 51.536 | 0.008 |
| NIPA1 | 11.189 | 10.862 | 0.025 |
| NPC1 | 5.515 | 6.062 | 0.001 |
| NRXN1 | 0.085 | 0.125 | 0.007 |
| NTNG1 | 0.327 | 0.249 | 0.009 |
| OTUD5 | 29.629 | 29.255 | 0.024 |
| OTUD6B | 7.405 | 6.534 | 0.048 |
| PACRG | 1.720 | 1.797 | 0.027 |
| PARD3B | 0.986 | 1.168 | 0.002 |
| PDE10A | 3.855 | 3.147 | 0.030 |
| PDE11A | 0.366 | 0.416 | 0.020 |
| PDGFRA | 11.237 | 11.624 | 0.009 |
| PFN1 | 386.845 | 384.213 | 0.010 |
| PHIP | 10.884 | 10.131 | 0.000 |
| PIGQ | 4.438 | 4.983 | 0.008 |
| PIH1D2 | 0.334 | 0.137 | 0.023 |
| PIK3CA | 12.209 | 9.685 | 0.057 |
| PINK1-AS | 1.974 | 1.689 | 0.029 |
| PLA2G6 | 0.634 | 0.830 | 0.016 |
| PMPCA | 13.790 | 11.969 | 0.022 |
| PNKP | 12.578 | 13.753 | 0.015 |
| PODXL | 4.194 | 4.847 | 0.011 |
| POLR1C | 7.525 | 6.234 | 0.034 |
| PPP2R2B | 0.179 | 0.146 | 0.056 |
| PRDX1 | 137.725 | 123.771 | 0.007 |
| PRICKLE1 | 3.757 | 3.333 | 0.002 |
| PRICKLE2 | 0.060 | 0.085 | 0.007 |
| PRKAR1B | 13.838 | 14.889 | 0.000 |
| PRPH | 0.870 | 1.132 | 0.011 |
| PRSS23 | 0.196 | 0.193 | 0.022 |
| PSEN2 | 5.259 | 5.579 | 0.051 |
| PTPRM | 4.505 | 4.710 | 0.000 |
| RAB33A | 6.793 | 6.922 | 0.003 |
| RBM12 | 3.638 | 3.884 | 0.000 |
| RELN | 9.086 | 8.830 | 0.007 |
| RGS4 | 28.376 | 23.445 | 0.019 |
| RHOBTB2 | 4.522 | 5.349 | 0.012 |
| RNF213 | 4.801 | 5.276 | 0.002 |
| RNF216 | 10.517 | 11.360 | 0.005 |
| ROGDI | 2.747 | 3.041 | 0.055 |
| SAMD12 | 0.011 | 0.002 | 0.052 |
| SDHA | 10.591 | 10.690 | 0.002 |
| SDHAF1 | 16.174 | 17.490 | 0.024 |
| SERPINI1 | 12.857 | 12.924 | 0.025 |
| SETD1B | 2.501 | 3.039 | 0.005 |
| SIL1 | 1.736 | 1.886 | 0.021 |
| SLC1A1 | 0.314 | 0.482 | 0.003 |
| SLC25A24 | 6.258 | 5.685 | 0.036 |
| SLC2A1 | 8.472 | 9.539 | 0.018 |
| SLC9B1 | 0.735 | 0.713 | 0.053 |
| SMARCA2 | 2.546 | 2.658 | 0.044 |
| SMARCC1 | 21.060 | 23.108 | 0.050 |
| SNHG1 | 19.818 | 17.384 | 0.004 |
| SORCS1 | 24.655 | 27.879 | 0.014 |
| SORL1 | 0.455 | 0.683 | 0.009 |
| SPAST | 6.944 | 5.978 | 0.053 |
| SPG21 | 15.666 | 17.306 | 0.035 |
| SPG7 | 2.790 | 2.471 | 0.011 |
| SQSTM1 | 32.378 | 38.955 | 0.007 |
| SREBF1 | 6.248 | 8.317 | 0.024 |
| STARD7 | 27.791 | 25.690 | 0.022 |
| SYCE2 | 0.567 | 0.604 | 0.049 |
| TBK1 | 15.661 | 12.891 | 0.052 |
| TCF4 | 32.383 | 36.707 | 0.017 |
| TCIRG1 | 0.302 | 0.340 | 0.023 |
| TFG | 31.479 | 30.210 | 0.015 |
| TIA1 | 24.343 | 18.552 | 0.029 |
| TMEM106B | 7.108 | 6.957 | 0.005 |
| TMEM230 | 40.014 | 35.809 | 0.054 |
| TMEM63A | 0.954 | 1.141 | 0.055 |
| TMLHE | 1.915 | 2.188 | 0.004 |
| TNF | 0.032 | 0.026 | 0.046 |
| TOMM40 | 13.758 | 15.441 | 0.001 |
| TP53 | 10.247 | 10.184 | 0.010 |
| TPP1 | 6.541 | 7.171 | 0.009 |
| TRAPPC2 | 3.872 | 4.012 | 0.053 |
| TRAPPC9 | 3.776 | 4.781 | 0.021 |
| TRPM7 | 7.393 | 5.958 | 0.030 |
| TTC21B | 4.351 | 4.248 | 0.026 |
| TUBA4A | 0.334 | 0.432 | 0.053 |
| TUBB4A | 7.889 | 12.522 | 0.013 |
| TUNAR | 0.382 | 0.441 | 0.009 |
| TWNK | 22.506 | 19.867 | 0.010 |
| UBE4A | 12.586 | 11.589 | 0.050 |
| UBQLN2 | 10.132 | 10.681 | 0.055 |
| UNC5C | 33.422 | 34.253 | 0.001 |
| USP8 | 4.840 | 3.827 | 0.047 |
| VAPB | 5.611 | 5.235 | 0.005 |
| VCP | 49.074 | 49.391 | 0.025 |
| VPS13D | 4.370 | 4.565 | 0.001 |
| WDR37 | 7.709 | 7.870 | 0.023 |
| ZFYVE26 | 1.909 | 1.930 | 0.032 |
| ZNF804A | 0.395 | 0.421 | 0.044 |

1. A method for the treatment or prevention of at least one neurodegenerative disorder and/or dementia, the method comprising administering to a subject in need thereof: (i) cannabidiol (CBD) or a pharmaceutically acceptable salt or derivative thereof, and (ii) an acetylcholinesterase inhibitor, and wherein the subject is administered from about 1 mg to about 1500 mg CBD or a pharmaceutically acceptable salt or derivative thereof per day.

2. The method of claim 1, wherein the subject is administered from about 1 mg to about 50 mg of the acetylcholinesterase inhibitor per day.

3. The method of claim 1, wherein the acetylcholinesterase inhibitor is selected from the group consisting of donepezil or a pharmaceutically acceptable salt thereof, rivastigmine or a pharmaceutically acceptable salt thereof, and galantamine or a pharmaceutically acceptable salt thereof.

4. The method of claim 3, wherein the acetylcholinesterase inhibitor is donepezil or a pharmaceutically acceptable salt thereof.

5. (canceled)

6. The method of claim 1, wherein:
(a) the subject is administered from about 20 mg to about 50 mg CBD or a pharmaceutically acceptable salt or derivative thereof per day; or
(b) the subject is administered from about 50 mg to about 500 mz CBD or a pharmaceutically acceptable salt or derivative thereof per day; or
(c) the subject is administered from about 500 mg to about 1500 mg CBD or a pharmaceutically acceptable salt or derivative thereof per day.

7. (canceled)

8. (canceled)

9. The method of claim 1, wherein one or both of the following apply:
(a) the CBD or a pharmaceutically acceptable salt or derivative thereof and the acetylcholinesterase inhibitor are orally administered to the subject; and
(b) the CBD or a pharmaceutically acceptable salt or derivative thereof and the acetylcholinesterase inhibitor are provided to the subject as a single daily dose.

10. The method of claim 1, wherein the dementia is selected from the group consisting of mild dementia, moderate dementia and severe dementia.

11. The method of claim 1, wherein the dementia is selected from the group consisting of vascular dementia, dementia with Lewy bodies, frontotemporal dementia, HIV-induced dementia, and traumatic brain injury (TBI) with dementia.

12. The method of claim 1, wherein the at least one neurodegenerative disorder selected from the group consisting of Alzheimer's disease, Parkinson's disease, Huntington's disease, chronic traumatic encephalopathy (CTE), amyotrophic lateral sclerosis (ALS), neurodegeneration with brain iron accumulation, mitochondrial dysfunction, Huntington's disease, ataxia, and neuronal ceroid lipofuscinosis (NCL).

13. The method of claim 12, wherein the at least one neurodegenerative disorder is selected from the group consisting of Parkinson's disease, Alzheimer's disease, Huntington's disease and ALS.

14. The method of claim 13, wherein the at least one neurodegenerative disorder is Alzheimer's disease.

15. The method of claim 1, wherein the CBD or a pharmaceutically acceptable salt or derivative thereof and the acetylcholinesterase inhibitor are sequentially or simultaneously administered.

16. (canceled)

17. (canceled)

18. (canceled)

19. A kit comprising CBD or a pharmaceutically acceptable salt or derivative thereof, and an acetylcholinesterase inhibitor, wherein the kit comprises the CBD in a dosage form comprising from about 1 mg to about 1500 mg CBD or a pharmaceutically acceptable salt or derivative thereof per dose.

20. A pharmaceutical composition comprising CBD or a pharmaceutically acceptable salt or derivative thereof, and an acetylcholinesterase inhibitor, wherein the pharmaceutical composition comprises from about 1 mg to about 1500 mg CBD or a pharmaceutically acceptable salt or derivative thereof per dose.

21. The pharmaceutical composition of claim 20, further comprising one or more pharmaceutically acceptable carriers, diluents and excipients.

22. The pharmaceutical composition of claim 20, comprising from about 1 mg to about 50 mg of the acetylcholinesterase inhibitor per dose.

23. The pharmaceutical composition of claim 20, wherein the acetylcholinesterase inhibitor is selected from the group consisting of donepezil or a pharmaceutically acceptable salt thereof, rivastigmine or a pharmaceutically acceptable salt thereof, and galantamine or a pharmaceutically acceptable salt thereof.

24. The pharmaceutical composition of claim 23, wherein the acetylcholinesterase inhibitor is donepezil or a pharmaceutically acceptable salt thereof.

25. The pharmaceutical composition of claim 20, comprising:
(a) from about 20 mg to about 50 mg CBD or a pharmaceutically acceptable salt or derivative thereof per dose
(b) from about 50 mg to about 500 mg CBD or a pharmaceutically acceptable salt or derivative thereof per dose; or
(c) from about 500 mg to about 1500 mg CBD or a pharmaceutically acceptable salt or derivative thereof per dose.

26. (canceled)

27. (canceled)

28. The pharmaceutical composition of claim 20, which is for oral administration.

29. (canceled)

30. (canceled)

31. (canceled)

32. (canceled)

33. (canceled)
